SEQUENCE LISTING (19 sequences)

SEQ ID NO: 1 - DNA, 1642 bp, Rattus norvegicus
agccttgccgagcctggcttggttggaaggaggaggatgctctgcgtgcccagggtcctgtgggcactgacatccaacgtactcatgcccatttcctgtaatggacagacttgatgctaatgtgagttccaacgagggtttcgggtctgtggagaaggtcgtactgctcacgttcttcgcaatggttatcctgatggccatcctgggcaacctgctggtgatggttgctgtgtgcagggacaggcagctcaggaaaataaaaaccaattatttcattgtgtctcttgcctttgctgatctgctggtttcggtgctggtgaatgccttcggtgccattgagttggtccaagacatctggttttatggggagatgttttgcctggtccggacctctctggatgtcctactcaccacagcatcaatttttcacctctgctgcatttccctggataggtattatgccatctgctgtcaacctttggtttatagaaacaagatgacccctctacgcatcgcattaatgctgggaggctgctgggtcattcccatgtttatatcttttctccccataatgcaaggctggaacaacatcggcatagttgatgtgatagagaaaaggaaattcaaccacaactctaactctacattctgtgtcttcatggtcaacaagccctatgccatcacctgctctgtggtggccttctacatcccgtttctcctcatggtgctggcctattaccgtatctatgtcactgctaaggagcatgcccagcagatccagatgttacaacgggcaggagccacctctgaaagcaggccccagacagctgaccagcacagcacacatcgcatgcggacagagaccaaagcagccaagactttatgtgtcatcatgggctgcttctgtttctgctgggcccccttctttgtcaccaatattgtggaccctttcatagactacactgtgcccgagaaggtgtggactgctttcctctggcttggctatatcaattcagggttgaacccttttctctatgccttcttgaataagtctttcagacgtgccttccttatcatcctctgctgtgatgatgagcgctacaaaagaccccccattctgggccagactgtcccctgttcaaccacaaccattaatggatccactcatgtgctaaggtatacagttttgcatagtggtcaacaccaggaactggagaagttgcccatacacaatgacccagagtccctggaatcatgcttttgattgaagacgtggcttgcctttagcggttcatcccatctgtgtctgcatgaacaggttactatggaatcactcctgactctgggcatcaccagtgaagcatgagcatggtgaggcagggtccggtgaaggtgcacagaggacagcattgagtgggacctgaacccagcacattaaggatttcagaaccgtgtggggatttgagatgtcatcagacccagtgtcttacccagagcccaactggcacctcccattccacgctgacatgtggtcagtctttgctcacacctctccaggggcaggagctgactacctcctaatgtggtggggagctcttaattgtgtggaagttcagtcattcattggtggacagtctcgctg

SEQ ID NO: 2 - protein, 387 aa, Rattus norvegicus
MDRLDANVSSNEGFGSVEKVVLLTFFAMVILMAILGNLLVMVAVCRDRQLRKIKTNYFIVSLAFADLLVSVLVNAFGAIELVQDIWFYGEMFCLVRTSLDVLLTTASIFHLCCISLDRYYAICCQPLVYRNKMTPLRIALMLGGCWVIPMFISFLPIMQGWNNIGIVDVIEKRKFNHNSNSTFCVFMVNKPYAITCSVVAFYIPFLLMVLAYYRIYVTAKEHAQQIQMLQRAGATSESRPQTADQHSTHRMRTETKAAKTLCVIMGCFCFCWAPFFVTNIVDPFIDYTVPEKVWTAFLWLGYINSGLNPFLYAFLNKSFRRAFLIILCCDDERYKRPPILGQTVPCSTTTINGSTHVLRYTVLHSGQHQELEKLPIHNDPESLESCF

SEQ ID NO: 3 - DNA, 1622 bp, Rattus norvegicus
agggtcctgtgggcactgacatccaacgtactcatgcccatttcctgtaatggacagacttgatgctaatgtgagttccaacgagggtttcgggtctgtggagaaggtcgtactgctcacgttcttcgcaatggttatcctgatggccatcctgggcaacctgctggtgatggttgctgtgtgcagggacaggcagctcaggaaaataaaaaccaattatttcattgtgtctcttgcctttgctgatctgctggtttcggtgctggtgaatgccttcggtgccattgagttggtccaagacatctggttttatggggagatgttttgcctggtccggacctctctggatgtcctactcaccacagcatcaatttttcacctctgctgcctttccctggataggtattatgccatctgctgtcaacctttggtttatagaaacaagatgacccctctacgcatcgcattaatgctgggaggctgctgggtcattcccatgtttatatcttttctccccataatgcaaggctggaacaacatcggcatagttgatgtgatagagaaaaggaaattcaaccacaactctaactctacattctgtgtcttcatggtcaacaagccctatgccatcacctgctctgtggtggccttctacatcccgtttctcctcatggtgctggcctattaccgtatctatgtcactgctaaggagcatgcccagcagatccagatgttacaacgggcaggagccacctctgaaagcaggccccagacagctgaccagcacagcacacatcgcatgcggacagagaccaaagcagccaagactttatgtgtcatcatgggctgcttctgtttctgctgggcccccttctttgtcaccaatattgtggaccctttcatagactacactgtgcccgagaaggtgtggactgctttcctctggcttggctatatcaattcagggttgaacccttttctctatgccttcttgaataagtctttcagacgtgccttccttatcatcctctgctgtgatgatgagcgctacaaaagaccccccattctgggccagactgtcccctgttcaaccacaaccattaatggatccactcatgtgctaagggatacagtggaatgtggtggccaatgggagagtcggtgtcacctcacagcaacttctcctttggtggctgctcagccagtgatacgtaggccccaggacaatgacctagaagacagctgtagcttgaaaagaagccagtcctaagctgctacttcggtgtatgtggctgcccctggcactttgttctccaaggctttccaagagcatgaggcaatccaccctggacttcccgccacgattctagcaggcggtattagaggaagtcaggggagagaagggcttcctccttagctttctgtttctcaacattttctcttcctggagtctccactcttgcttggtggtctctgaagtccacgacccagtccccttttgctgtctccagtctgtcttgtaaatgtttaccgtgttcgattttcagtttccaaacatgccttctttgaagtgtcatcttacgatactgtcaaaacatgtgcctgtcttgatcacacttctt

SEQ ID NO: 4 - protein, 406 aa, Rattus norvegicus
MDRLDANVSSNEGFGSVEKVVLLTFFAMVILMAILGNLLVMVAVCRDRQLRKIKTNYFIVSLAFADLLVSVLVNAFGAIELVQDIWFYGEMFCLVRTSLDVLLTTASIFHLCCISLDRYYAICCQPLVYRNKMTPLRIALMLGGCWVIPMFISFLPIMQGWNNIGIVDVIEKRKFNHNSNSTFCVFMVNKPYAITCSVVAFYIPFLLMVLAYYRIYVTAKEHAQQIQMLQRAGATSESRPQTADQHSTHRMRTETKAAKTLCVIMGCFCFCWAPFFVTNIVDPFIDYTVPEKVWTAFLWLGYINSGLNPFLYAFLNKSFRRAFLIILCCDDERYKRPPILGQTVPCSTTTINGSTHVLRDTVECGGQWESRCHLTATSPLVAAQPVIRRPQDNDLEDSCSLKRSQS

SEQ ID NO: 5 - DNA, 536 bp, Homo sapiens
ttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatccgatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattc

SEQ ID NO: 6 - protein, 178 aa, Homo sapiens
LVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHPMRTETKAAKTLCIIMGCFCLCWAPFFVTNIVDPFIDYTVPGQVWTAFLWLGYIN

SEQ ID NO: 7 - DNA, 1316 bp, Homo sapiens
cctgtaatggacaaacttgatgctaatgtgagttctgaggagggtttcgggtcagtggagaaggtggtgctgctcacgtttctctcgacggttatcctgatggccatcttggggaacctgctggtgatggtggctgtgtgctgggacaggcagctcaggaaaataaaaacaaattatttcattgtatctcttgcttttgcggatctgctggtttcggtgctggtgatgccctttggtgccattgagctggttcaagacatctggatttatggggaggtgttttgtcttgttcggacatctctggacgtcctgctcacaacggcatcgatttttcacctgtgctgcatttctctggataggtattacgccatctgctgccagcctttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatccagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaagggatgcagtggagtgtggtggccagtgggagagtcagtgtcacccgccagcaacttctcctttggtggctgctcagcccagtgacacttaggcccctgggacaatgacccagaagacagccatgcctccgaaagagggccaggtcctaagctgctgcttgtgcgcgactgcacccggcattctcttcacctgaggctttccgtccgccagtgcaggaacccggtgctcgctggg

SEQ ID NO: 8 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 9 - DNA, 31 bp, Artificial Sequence
  Description of Artificial Sequence primer
tggaattctgygynathkcnctggaymgsta

SEQ ID NO: 10 - DNA, 27 bp, Artificial Sequence
  Description of Artificial Sequence primer
catnavnrnnarnggdatrwaraangc

SEQ ID NO: 11 - DNA, 45 bp, Artificial Sequence
  Description of Artificial Sequence probe
tcaaaagcatgattccagggactctgggtcattgtgtatgggcaa

SEQ ID NO: 12 - DNA, 25 bp, Artificial Sequence
  Description of Artificial Sequence primer
cctcaatcagaagcatgattccagg

SEQ ID NO: 13 - DNA, 25 bp, Artificial Sequence
  Description of Artificial Sequence primer
ttggtctataggaacaagatgaccc

SEQ ID NO: 14 - DNA, 792 bp, Homo sapiens
ttggtctataggaacaagatgacccctctgcgcatcgcattaatgctgggaggctgctgggtcatccccacgtttatttcttttctccctataatgcaaggctggaataacattggcataattgatttgatagaaaagaggaagttcaaccagaactctaactctacgtactgtgtcttcatggtcaacaagccctacgccatcacctgctctgtggtggccttctacatcccatttctcctcatggtgctggcctattaccgcatctatgtcacagctaaggagcatgcccatcagatcagatgttacaacgggcaggagcctcctccgagagcaggcctcagtcggcagaccagcatagcactcatcgcatgaggacagagaccaaagcagccaagaccctgtgcatcatcatgggttgcttctgcctctgctgggcaccattctttgtcaccaatattgtggatcctttcatagactacactgtccctgggcaggtgtggactgctttcctctggctcggctatatcaattccgggttgaacccttttctctacgccttcttgaataagtcttttagacgtgccttcctcatcatcctctgctgtgatgatgagcgctaccgaagaccttccattctgggccagactgtcccttgttcaaccacaaccattaatggatccacacatgtactaaggtacaccgttctgcacaggggacatcatcaggaactcgagaaactgcccatacacaatgacccagaatccctggaatcatgcttctgattgaggc

SEQ ID NO: 15 - protein, 261 aa, Homo sapiens
LVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRYTVLHRGHHQELEKLPIHNDPESLESCF

SEQ ID NO: 16 - protein, 445 aa, Rattus norvegicus
MMDVNSSGRPDLYGHLRSFLLPEVGRGLPDLSPDGGADPVAGSWAPHLLSEVTASPAPTWDAPPDNASGCGEQINYGRVEKVVIGSILTLITLLTIAGNCLVVISVCFVKKLRQPSNYLIVSLALADLSVAVAVMPFVSVTDLIGGKWIFGHFFCNVFIAMDVMCCTASIMTLCVISIDRYLGITRPLTYPVRQNGKCMAKMILSVWLLSASITLPPLFGWAQNVNDDKVCLISQDFGYTIYSTAVAFYIPMSVMLFMYYQIYKAARKSAAKHKFPGFPRVEPDSVIALNGIVKLQKEVEECANLSRLLKHERKNISIFKREQKAATTLGIIVGAFTVCWLPFFLLSTARPFICGTSCSCIPLWVERTFLWLGYANSLINPFIYAFFNRDLRTTYRSLLQCQYRNINRKLSAAGMHEALKLAERPERPEFVLQNADYCRKKGHDS

SEQ ID NO: 17 - protein, 471 aa, Rattus norvegicus
MEILCEDNISLSSIPNSLMQLGDGPRLYHNDFNSRDANTSEASNWTIDAENRTNLSCEGYLPPTCLSILHLQEKNWSALLTTVVIILTIAGNILVIMAVSLEKKLQNATNYFLMSLAIADMLLGFLVMPVSMLTILYGYRWPLPSKLCAIWIYLDVLFSTASIMNLCAISLDRYVAIQNPIHHSRFNSRTKAFLKIIAVWTISVGISMPIPVFGLQDDSKVFKEGSCLLADDNFVLIGSFVAFFIPLTIMVITYFLTIKSLQKEATLCVSDLSTRAKLASFSFLPQSSLSSEKLFQRSIHREPGSYAGRRTMQSISNEQKACKVLGIVFFLFVVMWCPFFITNIMAVICKESCNENVIGALLNVFVWIGYLSSAVMPLVYTLFMKTYRSAFSRYIQCQYKENRKPLQLILVNTIPALAYKSSQLQVGQKKNSQEDAEQTVDDCSMVTLGKQQSEENCTDNIETVNEKVSCV

SEQ ID NO: 18 - protein, 460 aa, Rattus norvegicus
MVNLGNAVRSLLMHLIGLLVWQFDISISPVAAIVTDTFNSSDGGRLFQFPDGVQNWPALSIVVIIINTIGGNILVIMAVSMEKKLHNATNTFLMSLAIADMLVGLLVMPLSLLAILYDYVWPLPRYLCPVWISLDVLFSTASIMNLCAISLDRYVAIRNPIEHSRFNSRTKAIMKIAIVWAISIGVSVPIPVIGLRDESKVFVNNTTCVLNDPNFVLIGSFVAFFIPLTIMVITYFLTIYVLRRQTLMLLRGHTEEELANMSLNFLNCCCKKNGGEEENAPNPNPDQKPRRKKKEKRPRGTMQAINNEKKASKVLGIVFFVFLIMWCPFFITNILSVLCGKACNQKLMEKLLNVFVWIGYVCSGINPLVYTLFNKIYRRAFSKYLRCDYKPDKKPPVRQIPRVAATALSGRELNVNIYRHENERVARKANDPEPGIEMQVENLELPVNPSNVVSERISSV

SEQ ID NO: 19 - protein, 359 aa, canine
MISNGTGSSFCLDSPPCRITVSVVLTVLILITIAGNVVVCLAVGLNRRLRSLTNCFIVSLAITDLLLGLLVLPFSAFYQLSCRWSFGKVFCNIYTSLDVMLCTASILNLFMISLDRYCAVTDPLRYPVLITPVRVAVSLVLIWVISITLSFLSIHLGWNSRNETSSFNHTIPKCKVQVNLVYGLVDGLVTFYLPLLVMCITYYRIFKIARDQAKRIHHMGSWKAATIGEHKATVTLAAVMGAFIICWFPYFTVFVYRGLKGDDAINEAFEAVVLWLGYANSALNPILYATLNRDFRTAYQQLFRCRPASHNAQETSLRSNSSQLARNQSREPMRQEEKPLKLQVWSGTEVTAPRGATDR

(19) United States
(12) Patent Application Publication
(10) Pub. No.: US 2002/0081661 A1
(43) Pub. Date: Jun. 27, 2002

(54) DNA encoding 5-HT4 serotonin receptors and uses thereof

(75) Inventors: Christophe P.G Gerald, Ridgewood, NJ (US); Paul R. Hartig, Pennington, NJ (US); Theresa A. Branchek, Teaneck, NJ (US); Richard L. Weinshank, Teaneck, NJ (US)

Correspondence Address: John P. White, Cooper & Dunham LLP, 1185 Avenue of the Americas, New York, NY, 10036, US

(73) Assignee: Synaptic Pharmaceutical Corporation

(21) Appl. No.: 09/989,861

(22) Filed: Nov. 19, 2001

Related U.S. Application Data
(63) Continuation of application No. 09/328,314, filed on Apr. 3, 1998.
Application No. 09/328,314 is a division of application No. 08/446,822, filed on Jul. 31, 1995.
Application No. 08/446,822 is a 371 of international application No. PCT/US93/12586, filed on Dec. 22, 1993.
Application No. 08/446,822 is a 371 of international application No. 07/996,772, filed on Dec. 24, 1992.

(51) Int. Cl. C07K 14/705; C12P 21/02; C12N 5/06; C07H 21/04

(52) U.S. Cl. 435/69.1; 435/320.1; 435/325; 530/350; 536/23.5

(57) ABSTRACT

This invention provides an isolated nucleic acid molecule encoding a mammalian 5-HT₄ receptor and an isolated nucleic acid molecule encoding a human 5-HT₄ receptor, an isolated protein which is a mammalian 5-HT₄ receptor, an isolated protein which is a human 5-HT₄ receptor, vectors comprising an isolated nucleic acid molecule encoding a mammalian 5-HT₄ receptor, vectors comprising and isolated nucleic acid molecule encoding a human 5-HT₄ receptor, mammalian cells comprising such vectors, antibodies directed to the 5-HT₄ receptor, nucleic acid probes useful for detecting nucleic acid encoding a mammalian or human 5-HT₄ receptor, antisense oligonucleotides complementary to any sequences of a nucleic acid molecule which encodes a mammalian or human 5-HT₄ receptor, pharmaceutical compounds related to the human 5-HT₄ receptor, and nonhuman transgenic animals which express DNA encoding a normal or a mutant mammalian or human 5-HT₄ receptor. This invention further provides methods for determining ligand binding, detecting expression, drug screening, and treatments for alleviating abnormalities associated with a human 5-HT₄ receptor.

### BACKGROUND OF THE INVENTION

Throughout this application various publications are referred to by partial citations within parenthesis. Full citations for these publications may be found at the end of the specification immediately preceding the claims. The disclosures of these publications, in their entireties, are hereby incorporated by reference into this application in order to more fully describe the state of the art to which this invention pertains.

Molecular cloning efforts have provided primary amino acid sequence and signal transduction data for a large collection of serotonin receptor subtypes. These include five cloned 5-HT₁-like receptors, three cloned 5-HT₂ receptors, and one 5-HT₃ receptor. The B-HT₁ subfamily includes: 5-HT_{1A} (Fargin, 1988; Kobilka, 1989), 5-HT_{1B}/5-HT_{1Dβ} (Weinshank et al., 1991; Demchyshyn et al., 1992; Jin et al., 1992; Adham et al., 1992; Maroteaux et al., 1992; Voight et al., 1991), 5-HT_{1Dα} (Branchek et al. 1991; Hamblin and Metcalf, 1991; Weinshank et al., 1992), 5-HT_{1E} (Levy et al., 1992; McAllister et al., 1992; Zgombick et al., 1992) and 5-HT_{IF} (Adh.am et al., 1993). All five have been shown to couple to the inhibition of adenylate cyclase activity. The 5-HT₂ family includes the 5-HT₂ receptor (Pritchett et al., 1988), 5-HT_{1C} (Julius et al., 1989) and 5-HT_{2F} (Rat Stomach Fundus; Foquet et al., 1992; Kursar et al., 1992). These receptors all couple to phosphoinositide hydrolysis. The 5-HT₃ receptor is a ligand-gated ion channel (Maricq et al., 1991).

Although this work represents enormous success, the absence of molecular biological information on the 5-HT₄ receptors, which have been shown in native tissues to couple to the activation of adenylate cyclase as a primary mode of signal transduction (Dumius et al., 1988; Bockaert et al., 1990), is apparent. In a previous copending application (U.S. Ser. No. 971,690, filed Nov. 3, 1992), we reported the cloning of the first mammalian 5-HT receptor that couples to the stimulation of adenylate cyclase activity which we named 5-HT_{4B}. The 5-HT_{4B} receptor was subsequently renamed to the “5-HT₇ receptor” by the “Serotonin Receptor Nomenclature Committee” of the IUPHAR. The pharmacological properties of this receptor indicated that it was similar to a series of functionally defined 5-HT receptors described in the porcine vena cava (Trevethick et al., 1984), cat saphenous vein, coronary arteries (Cushing and Cohen, 1992), and several vascular dilatory effects (Mylecharane and Phillips, 1989). However, the classically defined 5-HT₄ receptor remained to be cloned. We now report the cloning of the pharmacologically-defined 5-HT₄ receptor which we have previously called ⁵-HT_{4A} and now designate as the 5-HT₄ receptor. This receptor also stimulates adenylate cyclase activity but unlike 5-HT_{4B}, is sensitive to a series of benzamide derivatives which act as agonists or partial agonists at this subtype. The presence of this subtype in the brain, particularly in areas such as the hippocampus, indicates a potential role in cognitive enhancement. In addition, the 5-HT₄ receptor has been described functionally in the heart, adrenal, bladder, and alimentary canal indicating potential roles in achalasia, hiatal hernia, esophageal spasm, irritable bowel disease, postoperative ileus, diabetic gastroparesis, emesis and other diseases of the gastrointestinal tract, as well as in cardiac, urinary, and endocrine function.

### SUMMARY OF THE INVENTION

This invention provides an isolated nucleic acid molecule encoding a mammalian 5-HT₄ receptor. In a preferred embodiment of this invention, the isolated nucleic acid encodes a human 5-HT4 receptor. In another embodiment of this invention, the nucleic acid molecule encoding a human 5-HT₄ receptor comprises a plasmid designated pBluescript-hS10 (ATCC Accession No. 75392). In another embodiment of this invention a nucleic acid molecule encoding a mammalian 5HT4 receptor comprises a plasmid designated pcEXV-S10-87 (ATCC Accession No. 75390). In another embodiment of this invention a nucleic acid molecule encoding a mammalian 5-HT₄ receptor comprises a plasmid designated pcEXV-S10-95 (ATCC Accession No. 75391).

This invention provides a nucleic acid probe comprising a nucleic acid molecule of at least 15 nucleotides capable of specifically hybridizing with a unique sequence included within the sequence of a nucleic acid molecule encoding a mammalian 5-HT₄ receptor. This invention also provides a nucleic acid molecule of at least 15 nucleotides capable of specifically hybridizing with a sequence included within the sequence of a nucleic acid molecule encoding a human 5HT-₄ receptor.

This invention provides an antisense oligonucleotide having a sequence capable of binding specifically to an mRNA molecule encoding a mammalian 5-HT₄ receptor so as to prevent translation of the mRNA molecule. This invention also provides an antisense oligonucleotide having a sequence capable of binding specifically to an mRNA molecule encoding a human 5-HT₄ receptor so as to prevent translation of the mRNA molecule.

This invention provides a monoclonal antibody directed to a mammalian 5-HT₄ receptor. This invention also provides a monoclonal antibody directed to a human 5-HT₄ receptor.

This invention provides a pharmaceutical composition comprising an amount of a substance effective to alleviate the abnormalities resulting from overexpression of a mammalian 5-HT₄ receptor and a pharmaceutically acceptable carrier. This invention also provides a pharmaceutical composition comprising an amount of a substance effective to alleviate abnormalities resulting from underexpression of mammalian 5-HT₄ receptor and a pharmaceutically acceptable carrier.

This invention provides a pharmaceutical composition comprising an amount of a substance effective to alleviate the abnormalities resulting from overexpression of a human 5-HT₄ receptor and a pharmaceutically acceptable carrier. This invention also provides pharmaceutical composition comprising an amount of a substance effective to alleviate abnormalities resulting from underexpression of a human 5-HT₄ receptor and a pharmaceutically acceptable carrier.

This invention provides a transgenic, nonhuman mammal whose genome comprises DNA encoding a mammalian 5-HT₄ receptor so positioned within such genome as to be transcribed into antisense mRNA complementary to mRNA encoding the mammalian 5-HT₄ receptor and when hybridized to mRNA encoding the mammalian 5-HT₄ receptor, the complementary mRNA reduces the translation of the mRNA encoding the mammalian 5-HT₄ receptor.

This invention also provides a transgenic, nonhuman mammal whose genome comprises DNA encoding a human 5-HT₄ so positioned within such genome as to be transcribed into antisense mRNA complementary to mRNA encoding the human 5-HT₄ and when hybridized to mRNA encoding the human 5-HT₄, the complementary mRNA reduces the translation of the mRNA encoding the human 5-HT₄.

This invention provides a transgenic, nonhuman mammal whose genome comprises DNA encoding a mammalian 5-HT₄ receptor so positioned within such genome as to be transcribed into antisense mRNA which is complementary to mRNA encoding the mammalian 5-HT₄ receptor and when hybridized to mRNA encoding the 5-HT₄ receptor, the antisense mRNA thereby prevents the translation of mRNA encoding the 5-HT₄ receptor.

This invention also provides a transgenic, nonhuman mammal whose genome comprises DNA encoding a human 5-HT₄ receptor so positioned within such genome as to be transcribed into antisense mRNA which is complementary to mRNA encoding the human 5-HT₄ receptor and when hybridized to mRNA encoding the human 5-HT₄ receptor, the antisense mRNA thereby prevents the translation of mRNA encoding the human 5-HT₄ receptor.

This invention also provides a method of determining the physiological effects of expressing varying levels of a mammalian 5-HT₄ receptor which comprises producing a transgenic nonhuman animal whose levels of mammalian 5-HT₄ receptor expression are varied by use of an inducible promoter which regulates mammalian 5-HT₄ receptor expression.

This invention also provides a method of determining the physiological effects of expressing varying levels of a human 5-HT₄ receptor which comprises producing a transgenic nonhuman animal whose levels of human 5-HT₄ receptor expression are varied by use of an inducible promoter which regulates human 5-HT₄ receptor expression.

This invention further provides a method of determining the physiological effects of expressing varying levels of mammalian 5-HT₄ receptor which comprises producing a panel of transgenic nonhuman animals each expressing a different amount of mammalian 5-HT₄ receptor.

This invention further provides a method of determining the physiological effects of expressing varying levels of human 5-HT₄ receptor which comprises producing a panel of transgenic nonhuman animals each expressing a different amount of human 5-HT₄ receptor.

This invention provides a method for determining whether a compound not known to be capable of specifically binding to a human 5-HT₄ receptor can specifically bind to the human 5-HT₄ receptor, which comprises contacting a mammalian cell comprising a plasmid adapted for expression in a mammalian cell which plasmid further comprises a DNA which expresses a human 5-HT₄ receptor on the cell's surface with the compound under conditions permitting binding of ligands known to bind to a human 5-HT₄ receptor, detecting the presence of any compound bound to the human 5-HT₄ receptor, the presence of bound compound indicating that the compound is capable of specifically binding to the human 5-HT₄ receptor.

This invention provides a method of screening drugs to identify drugs which interact with, and specifically bind to, a human 5-HT₄ receptor on the surface of a cell, which comprises contacting a mammalian cell which comprises a plasmid adapted for expression in a mammalian cell which plasmid further comprises DNA which expresses a human 5-HT₄ receptor on the cell's surface with a plurality of drugs, determining those drugs which bind to the human 5-HT₄ receptor expressed on the cell surface of the mammalian cell, and thereby identifying drugs which interact with, and specifically bind to, the human 5-HT₄ receptor.

This invention provides a method for identifying a compound which specifically binds to and activates or blocks the activation of a human 5-HT₄ receptor on the surface of a mammalian cell, which comprises contacting the mammalian cell which comprises a plasmid adapted for expression in the mammalian cell such plasmid further comprising DNA which expresses the human 5-HT₄ receptor on the cell surface of the mammalian cell with the compound, determining whether the compound activates or blocks the activation of the human 5-HT₄ receptor and thereby identifying the compound as a compound which binds to, and activates or blocks the activation of the human 5-HT₄ receptor.

This invention provides a method for diagnosing a predisposition to a disorder associated with the expression of a human 5-HT₄ receptor allele which comprises: a.) obtaining DNA of subjects suffering from the disorder; b.) performing a restriction digest of the DNA with a panel of restriction enzymes; c.) electrophoretically separating the resulting DNA fragments on a sizing gel; d.) contacting the resulting gel with a nucleic acid probe capable of specifically hybridizing to DNA encoding a 5-HT₄ receptor and labelled with a detectable marker; e.) detecting labelled bands which have hybridized to the DNA encoding a 5-HT₄ receptor labelled with a detectable marker to create a unique band pattern specific to the DNA of subjects suffering from the disorder; f.) preparing DNA obtained for diagnosis by steps a-e; and g.) comparing the unique band pattern specific to the DNA of subjects suffering from the disorder from step e and the DNA obtained for diagnosis from step f to determine whether the patterns are the same or different and to diagnose thereby predisposition to the disorder if the patterns are the same.

### BRIEF DESCRIPTION OF FIGURES

FIG. 1: Nucleotide and corresponding amino acid sequence of the S10-87 cDNA clone. Only partial 5′ and 3′ untranslated sequences are shown.

FIG. 2: Nucleotide and corresponding amino acid sequence of the S10-95 cDNA clone. Only partial 5′ and 3′ untranslated sequences are shown.

FIG. 3: Comparison of amino acid sequences between clones S10-87 (top row) and S10-95 (bottom row). The overall homology is 96.7%.

FIG. 4: Comparison of the rat S10 receptor deduced amino acid sequences with those of other serotonin receptors and with the canine histamine H2 receptor. Solid bars, the seven putative membrane-spanning domains (TM I-VII). Shading, homologies between the S10 receptors and other receptors. Hp78, 5-HT4B or hp78a receptor (U.S. Ser. No. 971,960, filed, Nov. 3, 1992, copending).

FIG. 5: Nucleotide and amino acid sequences of the human S10 PCR clone. The numbering is given according to the rat S10-95 clone.

FIG. 6: Comparison of nucleotide sequences between the human PCR S10 clone and the rat S10 cDNA clone. Top row: human sequence, the numbering is given according to the rat S10 nucleotide sequence. The bottom row outlines differences in the rat sequence (overall homology: 90.7%).

FIG. 7: Comparison of deduced amino acid sequences between the Human S10 PCR clone and the rat S10 cDNA clone. Top row: human S10 sequence, the numbering is given according to the rat S10 amino acid sequence. The bottom row outlines differences in the rat sequence (overall homology: 92.3%).

FIG. 8: Comparison of binding affinities of key compounds at the S10 clone with adenylate cyclase functional responses obtained with mouse collicular neurons. A correlation plot was constructed between affinity constants of drugs for the S10 receptor with those obtained at a pharmacologically defined 5-HT₄ receptor. Binding values for the correlation were taken from table 1 and were expressed as the negative logarithm. Functional data were taken from Dumuis et al. (1988). The correlation coefficient calculated by linear regression was 0.96 indicating that the rank order of potency for the compounds was similar in both preparations.

FIG. 9: Stimulation of cAMP production by 5-HT in transiently transfected Cos-7 cells expressing the cloned rat 5-HT₄ (CG-7) receptor and antagonism by ICS 205930. cAMP measurements on intact cells were as described under Methods and Materials. Each data point represents the mean of triplicates from a single experiment representative of at least 2 others. The vertical bars indicate S.E.M. Data are presented as percent maximum cAMP released by 5-HT (basal CAMP release: 0.020±0.002 pmol/ml/10 min; maximum cAMP release: 0.42±0.03 pmol/ml/10 min).

FIG. 10: Stimulation of cAMP production by 5-HT in transiently transfected Cos-7 cells expressing the cloned rat 5-HT₄ (CG-8) receptor and antagonism by ICS 205930. cAMP measurements on intact cells were as described under Methods and Materials. Each data point represents the mean of triplicates from a single experiment representative of at least two others. The vertical bars indicate S.E.M. Data are presented as percent maximum cAMP released by 5-HT (basal cAMP release: 0.023±0.004 pmol/ml/10 min; maximum CAMP release: 0.57±0.04 pmol/ml/10 min).

FIG. 11A: Nucleotide sequence of the partial human S10-87 clone. Only partial 3′ untranslated sequences are shown (SEQ. ID NO. 14).

FIG. 11B: Deduced amino acid sequence encoded by the nucleotide sequence of FIG. 11A of the partial human S10-87 clone (SEQ. ID NO. 15).

FIG. 12: Comparison of the nucleotide sequences between the human (top row) and the rat S10-87 (bottom row) cDNA clones. The overall identity is 90.8%.

FIG. 13: Comparison of the deduced amino acid sequences between the human (top row) and the rat (bottom row) S10-87 receptors. The overall identity is 93.9%.

FIG. 14A: Nucleotide sequence of the full length human S10-95 clone (SEQ. ID NO. 7).

FIG. 14B: Deduced amino acid sequence encoded by the nucleotide sequence of FIG. 14A (SEQ. ID NO. 8).

FIG. 15: Comparison of the nucleotide sequences between the human (top row) and the rat (bottom row) S10-95 cDNA clones. The overall identity is 90.7%.

FIG. 16: Comparison of the deduced amino acid sequences between the human (top row) and the rat (bottom row) S10-95 receptors. The overall identity is 93.8%.

FIG. 17: Comparison of the nucleotide sequences corresponding to the available coding regions between the two human isoforms (top row S10-95; bottom row S10-87) of the 5-HT₄ receptor. The overall identity is 92%. 87) FIG. 18: Comparison of the deduced amino acid sequences between the two human isoforms (top row S10-95; bottom row S10-87) of the 5-HT₄ receptor. The overall identity is 90%.

FIG. 19: Inhibition of [³H]GR11380 binding on the cloned rat CG-8 receptor by 5-HT, in the absence and presence of Gpp(NH)p (100 μM). Membranes harvested from transient transfectants (COS-7 cells) were incubated with [³H]IGR113808 (0.2-0.4 nM) for 30 min at 37° C. Nonspecific binding was defined by 50 μM unlabelled 5-HT. Data are from a single experiment. Data were analyzed by computer-assisted nonlinear regression analysis (Accufit; Lundon Software).

### DETAILED DESCRIPTION OF THE INVENTION

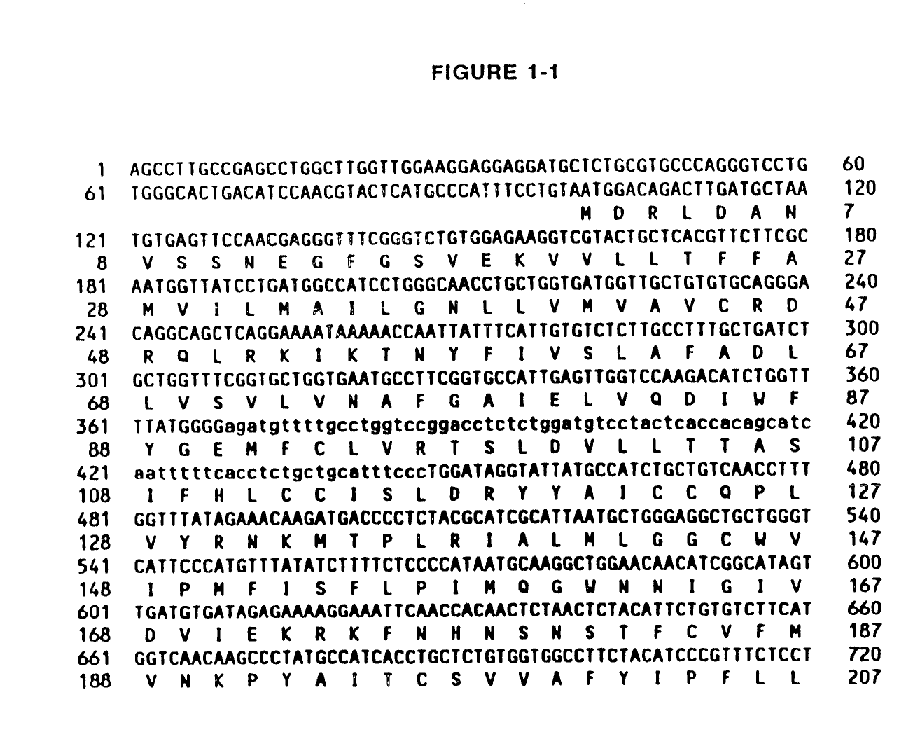

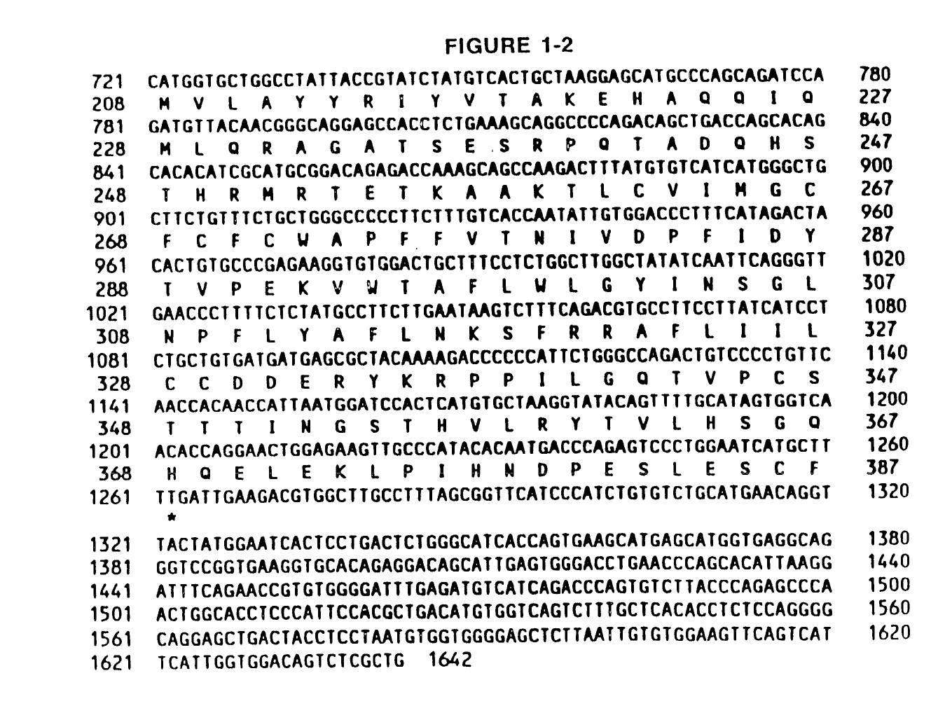

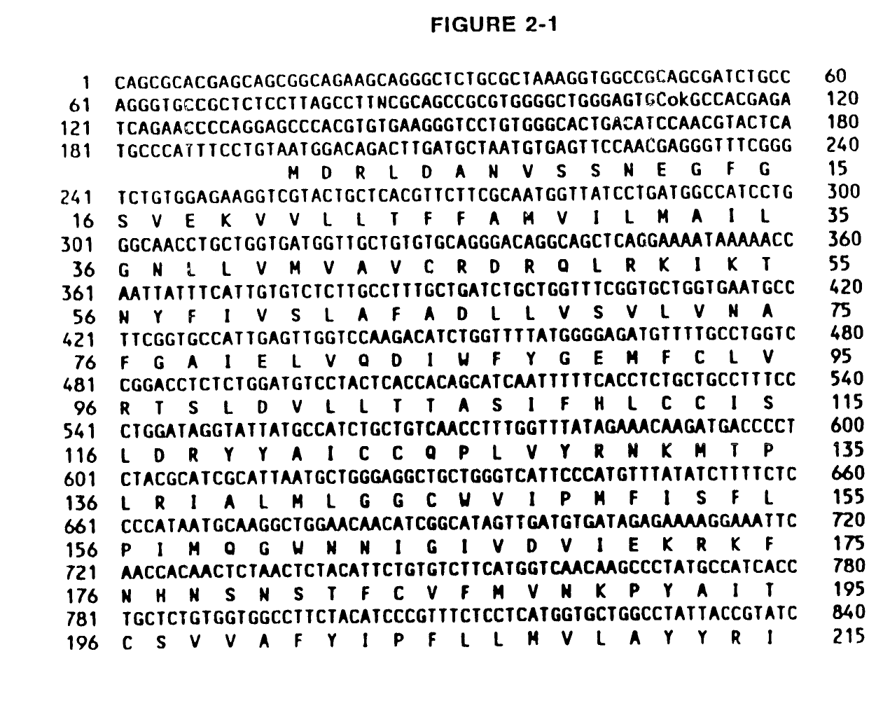

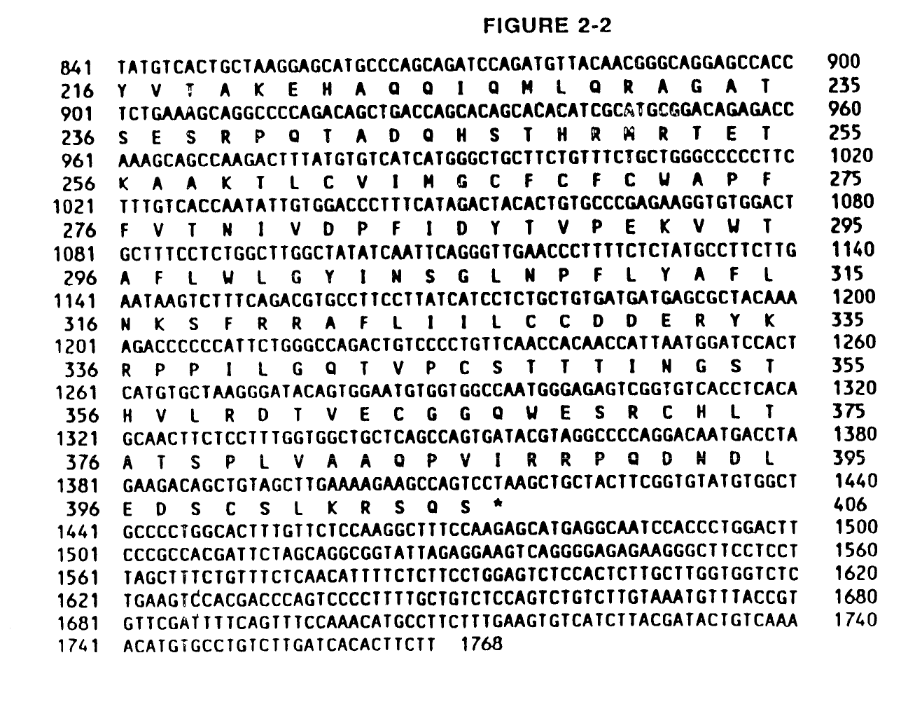

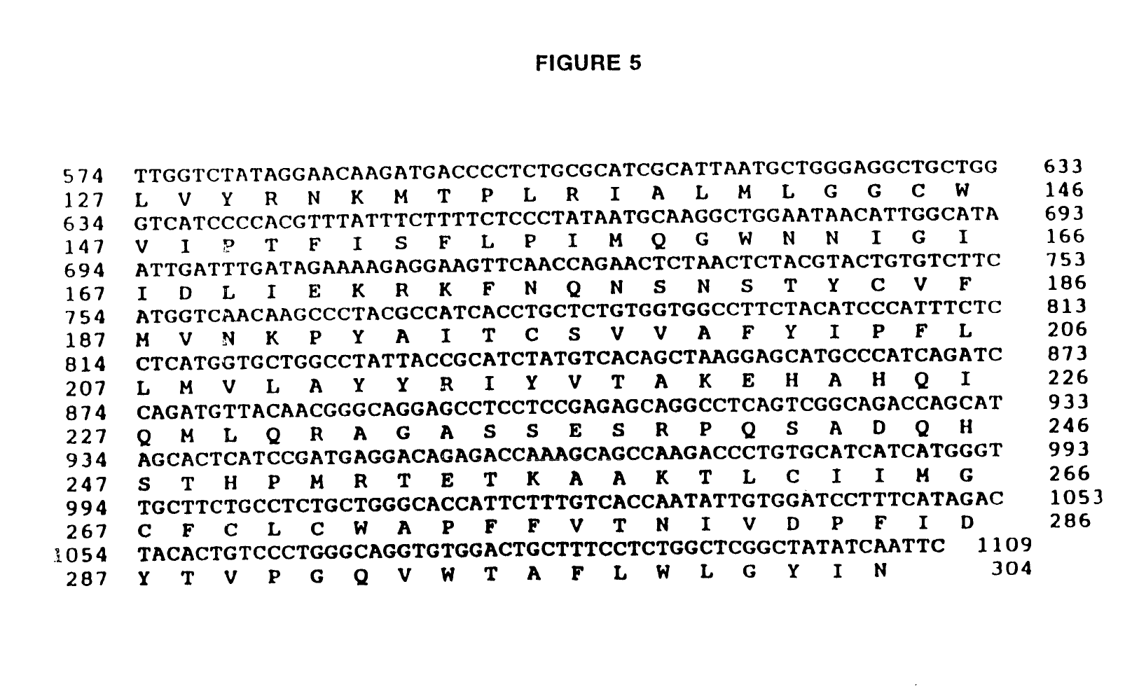

This invention provides an isolated nucleic acid molecule encoding a mammalian 5-HT₄ receptor. This invention further provides an isolated nucleic acid molecule encoding a human 5-HT₄ receptor. As used herein, the term “isolated nucleic acid molecule” means a non-naturally occurring nucleic acid molecule that is, a molecule in a form which does not occur in nature. Examples of such an isolated nucleic acid molecule are an RNA, cDNA, or isolated genomic DNA molecule encoding a mammalian 5-HT₄ receptor or a human 5-HT₄ receptor. As used herein, “5-HT₄ receptor” means a molecule which, under physiologic conditions, is substantially specific for the neurotransmitter serotonin, is saturable, of high affinity for serotonin and the activation of which is coupled to the activation of adenylate cyclase and the “5-HT₄ receptor” is also sensitive to benzamide derivatives which act as agonists and partial agonists at this receptor subtype. One embodiment of this invention is an isolated nucleic acid molecule encoding a mammalian 5-HT₄ receptor. Such a molecule may have coding sequences substantially the same as the coding sequences shown in FIGS. 1 and 2 and 5 (SEQ ID NOs. 1, 3 and 5). A preferred embodiment is an isolated nucleic acid molecule encoding a human 5-HT₄ receptor. Such a molecule may have a coding sequence substantially the same as the coding sequence shown in FIG. 5 (SEQ ID NO. 5). The DNA molecules of FIGS. 1, 2 and 5 (Seq ID NOs. 1, 3 and 5) encode the sequence of mammalian 5-HT₄ receptors. The DNA molecule of FIG. 5 (Seq ID No. 5) encodes a human 5-HT₄ receptor. This invention further provides isolated DNA molecules encoding mammalian 5-HT₄ receptors having the sequence H₂N—Y—X—COOH wherein Y is the amino acid sequence beginning at amino acid 1 and ending at amino acid 359 of FIG. 1 (SEQ ID NOs. 1 and 2) and wherein X is an amino acid sequence encoding the carboxy terminal region of the receptor. The nucleic acid molecules of FIGS. 1 and 2 (SEQ ID NOs 1-4) encode 5-HT₄ receptors having an identical sequence Y and differing only in their carboxy terminal region X beginning at amino acid 360. One means of isolating a nucleic acid molecule encoding a mammalian 5-HT₄ receptor is to probe a mammalian genomic library with a natural or artificially designed DNA probe, using methods well known in the art. In the preferred embodiment of this invention, the mammalian 5-HT₄ receptor is a human protein and the nucleic acid molecule encoding the human 5-HT₄ receptor is isolated from human cDNA. Degenerate oligonucleotide primers derived from transmembrane (TM) domains of 5-HT_{1A}, 5 -HT_{1C}, 5-HT₂ and 5-HT_{1Dα/B} receptors are useful for identifying cDNA containing a nucleic acid molecule encoding a 5-HT₄ receptor, obtaining a probe specific to a mammalian 5-HT₄ receptor and for isolating a nucleic acid molecule encoding a mammalian 5-HT₄ receptor.

DNA and cDNA molecules which encode a mammalian 5-HT₄ receptor are used to obtain complementary genomic DNA, cDNA or RNA from human, mammalian or other animal sources, or to isolate related cDNA or genomic clones by the screening of cDNA or genomic libraries, by methods described in more detail below. Transcriptional regulatory elements from the 5′ untranslated region of the isolated clone, and other stability, processing, transcription, translation, and tissue specificity determining regions from the 3′ and 5′ untranslated regions of the isolated gene are thereby obtained.

This invention provides an isolated nucleic acid molecule which has a nucleic acid sequence which differs from the sequence of a nucleic acid molecule encoding a 5-HT₄ receptor at one or more nucleotides and which does not encode a protein having 5-HT₄ receptor activity. As used herein, “5-HT₄ receptor activity” means the capability of receptor to specifically bind the neurotransmitter, serotonin under physiological conditions and the capability of the receptor to activate adenylate cyclase when the receptor is coupled to adenylate cyclase. An example of a isolated nucleic acid molecule provided by this invention is a nucleic acid molecule which has an in-frame stop codon inserted into the coding sequence such that the transcribed RNA is not translated into protein.

This invention further provides a cDNA molecule encoding a mammalian 5-HT₄ receptor, wherein the cDNA molecule has a coding sequence substantially the same as the coding sequence shown in FIGS. 1, 2 and 5 (Seq ID NOs. 1, 3 and 5). This invention provides a cDNA molecule encoding a human 5-HT₄ receptor, wherein the cDNA molecule has a coding sequence substantially the same as the coding sequence shown in FIG. 5 (SEQ ID NO. 5). These molecules and their equivalents were obtained by the means described above.

This invention also provides an isolated protein which is a mammalian 5-HT₄ receptor. In a preferred embodiment of this invention, the protein is a human 5-HT₄ receptor protein having an amino acid sequence substantially similar to the amino acid sequence shown in FIGS. 1, 2 and 5 (SEQ ID Nos. 1-6). In another embodiment of this invention, the protein is a murine 5-HT₄ receptor protein having an amino acid sequence substantially similar to the amino acid sequence shown in FIGS. 1, 2 and 5 (SEQ ID NOs. 1-6). As used herein, the term “isolated protein” is intended to encompass a protein molecule free of other cellular components. One means for obtaining an isolated mammalian 5-HT₄ receptor protein is to express DNA encoding the 5-HT₄ receptor in a suitable host, such as a bacterial, yeast, insect, or mammalian cell, using methods well known to those skilled in the art, and recovering the receptor protein after it has been expressed in such a host, again using methods well known in the art. The receptor may also be isolated from cells which express it, in particular from cells which have been transfected with the expression vectors described below in more detail.

This invention provides a vector comprising DNA, RNA, or cDNA, encoding a mammalian 5-HT₄ receptor. This invention further provides a vector comprising DNA, RNA, or cDNA, encoding a human 5-HT₄ receptor. Examples of vectors are viruses such as bacteriophages (such as phage lambda), cosmids, plasmids (such as pUCl8, available from Pharmacia, Piscataway, N.J.), and other recombination vectors. Nucleic acid molecules are inserted into vector genomes by methods well known to those skilled in the art. Examples of such plasmids are plasmids comprising DNA having a coding sequence substantially the same as the coding sequence shown in FIGS. 1, 2 and 5 (SEQ ID NOs. 1, 3 and 5) and designated pcEXV-S10-87 (ATCC Accession No. 75390), pcEXV-S10-95 (ATCC Accession No. 75391) and pBLuescript-hS10 (ATCC No. 75392).

Alternatively, to obtain these vectors, insert and vector DNA can both be exposed to a restriction enzyme to create complementary ends on both molecules which base pair with each other and are then ligated together with a ligase. Alternatively, linkers can be ligated to the insert DNA which correspond to a restriction site in the vector DNA, which is then digested with the restriction enzyme which cuts at that site. Other means are also available.

This invention also provides vectors comprising a DNA or cDNA encoding a mammalian 5-HT₄ receptor and vectors comprising a DNA or cDNA encoding a human 5-HT₄ receptor, adapted for expression in a bacterial cell, a yeast cell, insect cell or a mammalian cell which additionally comprise the regulatory elements necessary for expression of the DNA or cDNA encoding a mammalian 5-HT₄ receptor or the DNA or cDNA encoding a human 5-HT₄ receptor in the bacterial, yeast, insect or mammalian cells operatively linked to the DNA or cDNA encoding the 5-HT₄ receptor as to permit expression thereof. DNA or cDNA having coding sequence substantially the same as the coding sequence shown in FIGS. 1 and 2 (SEQ ID NOs. 1 and 3) may be usefully inserted into these vectors to express a mammalian 5-HT₄ receptor. DNA or cDNA having a coding sequence substantially the same as the coding sequence shown in FIG. 5 (SEQ ID NO. 5) may be usefully inserted into these vectors to express the human 5-HT₄ receptor. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Maniatis, et al., 1982). Similarly, a eukaryotic expression vector includes a heterologous or homologous promoter for RNA polymerase II, a downstream polyadenylation signal, the start codon AUG, and a termination codon for detachment of the ribosome. Furthermore, an insect expression vector, such as recombinant Baculovirus, uses the polyhedrin gene expression signals for expression of the inserted gene in insect cells. Such vectors may be obtained commercially or assembled from the sequences described by methods well known in the art, for example the methods described above for constructing vectors in general. Expression vectors are useful to produce cells that express receptors. Certain uses for such cells are described in more detail below.

In one embodiment of this invention a plasmid is adapted for expression in a bacterial, yeast, insect, or, in particular, a mammalian cell wherein the plasmid comprises DNA or cDNA encoding a mammalian 5-HT₄ receptor or DNA or cDNA encoding a human 5-HT₄ receptor and the regulatory elements necessary for expression of the DNA in the bacterial, yeast, insect, or mammalian cell operatively linked to the DNA or cDNA encoding a mammalian 5-HT₄ receptor or to the DNA or cDNA encoding a human 5-HT₄ receptor as to permit expression thereof. Suitable plasmids may include, but are not limited to plasmids adapted for expression in a mammalian cell, e.g., EVJB, EXV-3. An example of such a plasmid adapted for expression in a mammalian cell is a plasmid comprising cDNA having coding sequences substantially the same as the coding sequence shown in FIGS. 1, 2 and 5 (SEQ ID NOs. 1, 3 and 5) and the regulatory elements necessary for expression of the DNA in the mammalian cell. These plasmids have been designated pcEXV-S10-87 deposited under ATCC Accession No. 75390, pcEXV-S10-95 deposited under ATCC Accession No. 75391, and pBluescript-hS10, deposited under ATCC Accession No 75392. Those skilled in the art will readily appreciate that numerous plasmids adapted for expression in a mammalian cell which comprise DNA encoding a mammalian or human 5-HT₄ receptor and the regulatory elements necessary to express such DNA in the mammalian cell may be constructed utilizing existing plasmids and adapted as appropriate to contain the regulatory elements necessary to express the DNA in the mammalian cell. The plasmids may be constructed by the methods described above for expression vectors and vectors in general, and by other methods well known in the art.

Deposit discussed supra were made pursuant to, and in satisfaction of, the provisions of the Budapest Treaty on the International Recognition of the Deposit of Microorganisms for the Purpose of Patent Procedure with the American Type Culture Collection (ATCC), 12301 Parklawn Drive, Rockville, Md. 20852.

This invention provides a mammalian cell comprising a DNA or cDNA molecule encoding a mammalian 5-HT₄ receptor, such as a mammalian cell comprising a plasmid adapted for expression in a mammalian cell, said plasmid further comprises DNA or cDNA encoding a mammalian 5-HT₄ receptor and the regulatory elements necessary for expression of the DNA or cDNA in the mammalian cell operatively linked to the DNA or cDNA encoding a mammalian 5-HT₄ receptor as to permit expression thereof. This invention provides a mammalian cell comprising a DNA or cDNA molecule encoding a human 5-HT₄ receptor, such as a mammalian cell comprising a plasmid adapted for expression in a mammalian cell, said plasmid further comprises a DNA or cDNA molecule encoding a human 5-HT₄ receptor and the regulatory elements necessary for expression of the DNA or cDNA in the mammalian cell operatively linked to the DNA or cDNA encoding a human 5-HT₄ receptor as to permit expression thereof. Numerous mammalian cells may be used as hosts, including, but not limited to, the mouse fibroblast cell NIH3T3, CHO cells, HeLa cells, LM (tk−) cells, Cos-7 cells, etc. Expression plasmids such as that described supra may be used to transfect mammalian cells by methods well known in the art such as calcium phosphate precipitation, or DNA or cDNA encoding a human or mammalian 5-HT₄ receptor may be otherwise introduced into mammalian cells, e.g., by microinjection, to obtain mammalian cells which comprise DNA, e.g., cDNA or a plasmid, encoding a human or mammalian 5-HT₄ receptor.

This invention provides a nucleic acid probe comprising a nucleic acid molecule of at least 15 nucleotides capable of specifically hybridizing with an unique sequence included within the sequence of a nucleic acid molecule encoding a human 5-HT₄ receptor, for example with a coding sequence included within the sequences shown in FIG. 5 (SEQ ID NO. 5). This invention further provides a nucleic acid probe comprising a nucleic acid molecule of at least 15 nucleotides capable of specifically hybridizing with a sequence included within the sequence of a nucleic acid molecule encoding a mammalian 5-HT₄ receptor, for example with a coding sequence included within the sequences shown in FIG. 1 and FIG. 2 (SEQ ID NOs. 1 and 3) As used herein, the phrase “specifically hybridizing” means the ability of a nucleic acid molecule to recognize a nucleic acid sequence complementary to its own and to form double-helical segments through hydrogen bonding between complementary base pairs. As used herein, the phrase “unique sequence” means a nucleic acid molecule sequence specific to only the nucleic acid molecule encoding a mammalian 5-HT₄ receptor. Nucleic acid probe technology is well known to those skilled in the art who will readily appreciate that such probes may vary greatly in length and may be labeled with a detectable label, such as a radioisotope or fluorescent dye, to facilitate detection of the probe. Detection of nucleic acid encoding a human 5-HT₄ receptor is useful as a diagnostic test for any disease process in which levels of expression of the 5-HT₄ receptor are altered. DNA probe molecules are produced by insertion of a DNA molecule which encodes a 5-HT₄ receptor or fragments thereof into suitable vectors, such as plasmids or bacteriophages, followed by insertion into suitable bacterial host cells and replication and harvesting of the DNA probes, all using methods well known in the art. For example, the DNA may be extracted from a cell lysate using phenol and ethanol, digested with restriction enzymes corresponding to the insertion sites of the DNA into the vector (discussed above), electrophoresed, and cut out of the resulting gel. An example of such DNA molecules is shown in FIGS. 1, 2 and 5 (SEQ ID NOs. 1, 3, and 5)>. The probes are useful for ‘in situ’ hybridization or in order to locate tissues which express this gene family, or for other hybridization assays for the presence of these genes or their mRNA in various biological tissues. In addition, synthesized oligonucleotides (produced by a DNA synthesizer) complementary to the sequence of a DNA molecule which encode a mammalian 5-HT₄ receptor or complementary to the sequence of a DNA molecule which encodes a human 5-HT₄ receptor are useful as probes for these genes, for their associated mRNA, or for the isolation of related genes by homology screening of genomic or cDNA libraries, or by the use of amplification techniques such as the polymerase chain reaction.

This invention also provides a method of detecting expression of a human 5-HT₄ receptor on the surface of a cell by detecting the presence of mRNA coding for a 5-HT₄ receptor. This invention further provides a method of detecting expression of a mammalian 5-HT₄ receptor on the surface of the cell by detecting the presence of mRNA coding for a mammalian 5-HT₄ receptor. These methods comprise obtaining total mRNA from the cell using methods well known in the art and contacting the mRNA so obtained with a nucleic acid probe as described hereinabove, under hybridizing conditions, detecting the presence of mRNA hybridized to the probe, and thereby detecting the expression of the receptor by the cell. Hybridization of probes to target nucleic acid molecules such as mRNA molecules employs techniques well known in the art. However, in one embodiment of this invention, nucleic acids are extracted by precipitation from lysed cells and the mRNA is isolated from the extract using a column which binds the poly-A tails of the mRNA molecules (Maniatis et al., 1982). The mRNA is then exposed to radioactively labelled probe on a nitrocellulose membrane, and the probe hybridizes to and thereby labels complementary mRNA sequences. Binding may be detected by autoradiography or scintillation counting. However, other methods for performing these steps are well known to those skilled in the art, and the discussion above is merely an example.

This invention provides an antisense oligonucleotide having a sequence capable of binding specifically with any sequences of an mRNA molecule which encodes a human 5-HT₄ receptor so as to prevent translation of the human 5-HT₄ receptor. The antisense oligonucleotide may have a sequence capable of binding specifically with any sequences of the cDNA molecule whose sequence is shown in FIG. 5 (SEQ ID NO. 5). This invention also provides an antisense oligonucleotide having a sequence capable of binding specifically with any sequences of an mRNA molecule which encodes a mammalian 5-HT₄ receptor so as to prevent translation of the mammalian 5-HT₄ receptor. The antisense oligonucleotide may have a sequence capable of binding specifically with any sequences of the cDNA molecule whose sequence is shown in FIGS. 1 and 2 (SEQ ID NOs. 1 and 3). As used herein, the phrase “binding specifically” means the ability of an antisense oligonucleotide to recognize a nucleic acid sequence complementary to its own and to form double-helical segments through hydrogen bonding between complementary base pairs. A particular example of an antisense oligonucleotide is an antisense oligonucleotide comprising chemical analogues of nucleotides.

This invention also provides a pharmaceutical composition comprising an effective amount of the oligonucleotide described above effective to reduce expression of a human 5-HT₄ receptor by passing through a cell membrane and binding specifically with mRNA encoding the 5-HT₄ receptor in the cell so as to prevent its translation and a pharmaceutically acceptable hydrophobic carrier capable of passing through a cell membrane. This invention further provides a pharmaceutical composition comprising an effective amount of the oligonucleotide described above effective to reduce expression of a mammalian 5-HT₄ receptor by passing through a cell membrane and binding specifically with mRNA encoding a mammalian 5-HT₄ receptor in the cell so as to prevent its translation and a pharmaceutically acceptable hydrophobic carrier capable of passing through a cell membrane. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The oligonucleotide may be coupled to a substance which inactivates mRNA, such as a ribozyme. The pharmaceutically acceptable hydrophobic carrier capable of passing through cell membranes may also comprise a structure which binds to a transporter specific for a selected cell type and is thereby taken up by cells of the selected cell type. The structure may be part of a protein known to bind a cell-type specific transporter, for example an insulin molecule, which would target pancreatic cells. DNA molecules having a coding sequence substantially the same as the coding sequences shown in FIG. 5 (SEQ ID No. 5) may be used as the oligonucleotides of the pharmaceutical composition.

This invention also provides a pharmaceutical composition comprising an effective amount of the oligonucleotide described above effective to reduce expression of a mammalian 5-HT₄ receptor by passing through a cell membrane and binding specifically with mRNA encoding the 5-HT₄ receptor in the cell so as to prevent its translation and a pharmaceutically acceptable hydrophobic carrier capable of passing through a cell membrane. DNA molecules having a coding sequence substantially the same as the coding sequences shown in FIGS. 1 and 2 (SEQ ID NOs. 1 and 3) may be used as the oligonucleotides of the pharmaceutical composition.

This invention provides a method of treating abnormalities which are alleviated by reduction of expression of 5-HT₄ receptor. This method comprises administering to a subject an effective amount of the pharmaceutical composition described above effective to reduce expression of the 5-HT₄ receptor by the subject. This invention further provides a method of treating an abnormal condition related to 5-HT₄ receptor activity which comprises administering to a subject an amount of the pharmaceutical composition described above effective to reduce expression of the 5-HT₄ receptor by the subject. Examples of such abnormal conditions are irritable bowel disease, postoperative ileus, diabetic gastroparesis, emesis, achalasia, hiatal hernia, esophageal spasm and other diseases of the gastrointestinal tract, as well as in cardiac, urinary, and endocrine function.

Antisense oligonucleotide drugs inhibit translation of mRNA encoding 5-HT₄ receptor. Synthetic antisense oligonucleotides, or other antisense chemical structures are designed to bind to mRNA encoding the 5-HT₄ receptor and inhibit translation of mRNA and are useful as drugs to inhibit expression of 5-HT₄ receptor genes in patients. This invention provides a means to therapeutically alter levels of expression of a human or mammalian 5-HT₄ receptor by the use of a synthetic antisense oligonucleotide drug (SAOD) which inhibits translation of mRNA encoding the 5-HT₄ receptor. Synthetic antisense oligonucleotides, or other antisense chemical structures designed to recognize and selectively bind to mRNA, are constructed to be complementary to portions of the nucleotide sequence shown in FIGS. 1, 2 and 5 (SEQ ID NOs. 1, 3 and 5) of DNA, RNA or of chemically modified, artificial nucleic acids. The SAOD is designed to be stable in the blood stream for administration to patients by injection, or in laboratory cell culture conditions, for administration to cells removed from the patient. The SAOD is designed to be capable of passing through cell membranes in order to enter the cytoplasm of the cell by virtue of physical and chemical properties of the SAOD which render it capable of passing through cell membranes (e.g., by designing small, hydrophobic SAOD chemical structures) or by virtue of specific transport systems in the cell which recognize and transport the SAOD into the cell. In addition, the SAOD can be designed for administration only to certain selected cell populations by targeting the SAOD to be recognized by specific cellular uptake mechanisms which bind and take up the SAOD only within certain selected cell populations. For example, the SAOD may be designed to bind to transporter found only in a certain cell type, as discussed above. The SAOD is also designed to recognize and selectively bind to the target mRNA sequence, which may correspond to a sequence contained within the sequence shown in FIGS. 1, 2 and 5 (SEQ ID NOs. 1, 3, and 5) by virtue of complementary base pairing to the mRNA. Finally, the SAOD is designed to inactivate the target mRNA sequence by any of three mechanisms: 1) by binding to the target mRNA and thus inducing degradation of the mRNA by intrinsic cellular mechanisms such as RNAse I digestion, 2) by inhibiting translation of the mRNA target by interfering with the binding of translation-regulating factors or of ribosomes, or 3) by inclusion of other chemical structures, such as ribozyme sequences or reactive chemical groups, which either degrade or chemically modify the target mRNA. Synthetic antisense oligonucleotide drugs have been shown to be capable of the properties described above when directed against mRNA targets (Cohen, J. S., 1989; Weintraub, H. M., 1990). In addition, coupling of ribozymes to antisense oligonucleotides is a promising strategy for inactivating target mRNA (N. Sarver et al., 1990). An SAOD serves as an effective therapeutic agent if it is designed to be administered to a patient by injection, or if the patient's target cells are removed, treated with the SAOD in the laboratory, and replaced in the patient. In this manner, an SAOD serves as a therapy to reduce 5-HT₄ receptor expression in particular target cells of a patient, in any clinical condition which may benefit from reduced expression of 5-HT₄ receptor.

This invention provides an antibody directed to the human 5-HT₄ receptor. This invention also provides an antibody directed to the mammalian 5-HT₄ receptor. This antibody may comprise, for example, a monoclonal antibody directed to an epitope of a human 5-HT₄ receptor present on the surface of a cell, the epitope having an amino acid sequence substantially the same as an amino acid sequence for a cell surface epitope of the human 5-HT₄ receptor included in the amino acid sequence shown in FIG. 5. Amino acid sequences may be analyzed by methods well known to those skilled in the art to determine whether they produce hydrophobic or hydrophilic regions in the proteins which they build. In the case of cell membrane proteins, hydrophobic regions are well known to form the part of the protein that is inserted into the lipid bilayer which forms the cell membrane, while hydrophilic regions are located on the cell surface, in an aqueous environment. Therefore antibodies to the hydrophilic amino acid sequences shown in FIG. 5 will bind to a surface epitope of a 5-HT₄ receptor as described. Antibodies directed to a human or mammalian 5-HT₄ receptor may be serum-derived or monoclonal and are prepared using methods well known in the art. For example, monoclonal antibodies are prepared using hybridoma technology by fusing antibody producing B cells from immunized animals with myeloma cells and selecting the resulting hybridoma cell line producing the desired antibody. Cells such as NIH3T3 cells or LM (tk⁻) cells may be used as immunogens to raise such an antibody. Alternatively, synthetic peptides may be prepared using commercially available machines and the amino acid sequence shown in FIGS. 1, 2, and 5 (SEQ ID NOs. 1-6). As a still further alternative, DNA, such as a cDNA or a fragment thereof, may be cloned and expressed and the resulting polypeptide recovered and used as an immunogen. These antibodies are useful to detect the presence of 5-HT₄ receptor encoded by the isolated DNA, or to inhibit the function of the 5-HT₄ receptor in living animals, in humans, or in biological tissues or fluids isolated from animals or humans.

This invention also provides a pharmaceutical composition which comprises an effective amount of an antibody directed to an epitope of the human 5-HT₄ receptor, effective to block binding of naturally occurring substrates to the 5-HT₄ receptor, and a pharmaceutically acceptable carrier. A monoclonal antibody directed to an epitope of a human 5-HT₄ receptor present on the surface of a cell which has an amino acid sequence substantially the same as an amino acid sequence for a cell surface epitope of the human 5-HT₄ receptor included in the amino acid sequence shown in FIG. 5 (SEQ ID NOs. 5 and 6) is useful for this purpose.

This invention also provides a pharmaceutical composition which comprises an effective amount of an antibody directed to an epitope of a mammalian 5-HT₄ receptor, effective to block binding of naturally occurring substrates to the 5-HT₄ receptor, and a pharmaceutically acceptable carrier. A monoclonal antibody directed to an epitope of a mammalian 5-HT₄ receptor present on the surface of a cell which has an amino acid sequence substantially the same as an amino acid sequence for a cell surface epitope of a mammalian 5-HT₄ receptor included in the amino acid sequence shown in FIGS. 1 and 2 (SEQ ID NOs. 1-4) is useful for this purpose.

This invention also provides a method of treating abnormalities in a subject which are alleviated by reduction of expression of a human or mammalian 5-HT₄ receptor which comprises administering to the subject an effective amount of the pharmaceutical composition described above effective to block binding of naturally occurring substrates to the receptor and thereby alleviate abnormalities resulting from overexpression of a human or mammalian 5-HT₄ receptor. Binding of the antibody to the receptor prevents the receptor from functioning, thereby neutralizing the effects of overexpression. The monoclonal antibodies described above are useful for this purpose. This invention additionally provides a method of treating an abnormal condition related to an excess of 5-HT₄ receptor activity which comprises administering to a subject an amount of the pharmaceutical composition described above effective to block binding of naturally occurring substrates to the 5-HT₄ receptor and thereby alleviate the abnormal condition. Some examples of abnormal conditions associated with excess 5-HT₄ receptor activity are irritable bowel disease, postoperative ileus, diabetic gastroparesis, emesis, achalasia, hiatal hernia, esophageal spasm and other diseases of the gastrointestinal tract, as well as in cardiac, urinary, and endocrine function.

This invention provides methods of detecting the presence of a 5-HT₄ receptor on the surface of a cell which comprises contacting the cell with an antibody directed to the 5-HT₄ receptor, under conditions permitting binding of the antibody to the receptor, detecting the presence of the antibody bound to the cell, and thereby the presence of the 5-HT₄ receptor on the surface of the cell. Such methods are useful for determining whether a given cell is defective in expression of 5-HT₄ receptors. Bound antibodies are detected by methods well known in the art, for example by binding fluorescent markers to the antibodies and examining the cell sample under a fluorescence microscope to detect fluorescence on a cell indicative of antibody binding. The monoclonal antibodies described above are useful for this purpose.

This invention provides a transgenic nonhuman mammal expressing DNA encoding a human 5-HT₄ receptor and a transgenic nonhuman mammal expressing DNA encoding a mammalian 5-HT₄ receptor. This invention also provides a transgenic nonhuman mammal expressing DNA encoding a human or mammalian 5-HT₄ receptor so mutated as to be incapable of normal receptor activity, and not expressing native 5-HT₄ receptor. This invention further provides a transgenic nonhuman mammal whose genome comprises DNA encoding a human 5-HT₄ receptor so placed as to be transcribed into antisense mRNA which is complementary to mRNA encoding a human 5-HT₄ receptor and which hybridizes to mRNA encoding a 5-HT₄ receptor thereby reducing its translation and a transgenic nonhuman mammal whose genome comprises DNA encoding a mammalian 5-HT₄ receptor so placed as to be transcribed into antisense mRNA which is complementary to mRNA encoding a mammalian 5-HT₄ receptor and which hybridizes to mRNA encoding a mammalian 5-HT₄ receptor thereby reducing its translation. The DNA may additionally comprise an inducible promoter or additionally comprise tissue specific regulatory elements, so that expression can be induced, or restricted to specific cell types. Examples of DNA are DNA or cDNA molecules having a coding sequence substantially the same as the coding sequences shown in FIGS. 1,2 and 5 (SEQ ID NOs. 1, 3, and 5). An example of a transgenic animal is a transgenic mouse. Examples of tissue specificity-determining regions are the metallothionein promotor (Low et al., 1986) and the L7 promotor (Oberdick et al., 1990).

Animal model systems which elucidate the physiological and behavioral roles of mammalian receptors are produced by creating transgenic animals in which the expression of a receptor is either increased or decreased, or the amino acid sequence of the expressed receptor protein is altered, by a variety of techniques. Examples of these techniques include, but are not limited to: 1) Insertion of normal or mutant versions of DNA encoding a human 5-HT₄ receptor or homologous animal versions of this gene, by microinjection, retroviral infection or other means well known to those skilled in the art, into appropriate fertilized embryos in order to produce a transgenic animal (Hogan et al., 1986) or, 2) Homologous recombination (Capecchi M. R., 1989; Zimmer A, and Gruss, P., 1989) of mutant or normal, human or animal versions of these genes with the native gene locus in transgenic animals to alter the regulation of expression or the structure of the receptor. The technique of homologous recombination is well known in the art. It replaces the native gene with the inserted gene and so is useful for producing an animal that cannot express native receptor but does express, for example, an inserted mutant receptor, which has replaced the native receptor in the animal's genome by recombination, resulting in underexpression of the receptor. Microinjection adds genes to the genome, but does not remove them, and so is useful for producing an animal which expresses its own and added receptors, resulting in overexpression of the receptor.

One means available for producing a transgenic animal, with a mouse as an example, is as follows: Female mice are mated, and the resulting fertilized eggs are dissected out of their oviducts. The eggs are stored in an appropriate medium such as M2 medium (Hogan, B. et al. 1986). DNA or cDNA encoding a receptor is purified from a vector (such as plasmids pcEXV-S10-87, pcEXV-S10-95 and pBluescript-hS10 described above) by methods well known in the art. Inducible promoters may be fused with the coding region of the DNA to provide an experimental means to regulate expression of the trans-gene. Alternatively or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

Since the normal action of receptor-specific drugs is to activate or to inhibit the receptor, the transgenic animal model systems described above are useful for testing the biological activity of drugs directed against the receptors even before such drugs become available. These animal model systems are useful for predicting or evaluating possible therapeutic applications of drugs which activate or inhibit receptors by inducing or inhibiting expression of the native or trans-gene and thus increasing or decreasing expression of normal or mutant receptors in the living animal. Thus, a model system is produced in which the biological activity of drugs directed against the receptors are evaluated before such drugs become available. The transgenic animals which over or under produce the receptor indicate by their physiological state whether over or under production of the receptor is therapeutically useful. It is therefore useful to evaluate drug action based on the transgenic model system. One use is based on the fact that it is well known in the art that a drug such as an antidepressant acts by blocking neurotransmitter uptake, and thereby increases the amount of neurotransmitter in the synaptic cleft. The physiological result of this action is to stimulate the production of less receptor by the affected cells, leading eventually to underexpression. Therefore, an animal which underexpresses receptor is useful as a test system to investigate whether the actions of such drugs which result in under expression are in fact therapeutic. Another use is that if overexpression is found to lead to abnormalities, then a drug which down-regulates or acts as an antagonist to the receptor is indicated as worth developing, and if a promising therapeutic application is uncovered by these animal model systems, activation or inhibition of the 5-HT₄ receptor is achieved therapeutically either by producing agonist or antagonist drugs directed against the 5-HT₄ receptor or by any method which increases or decreases the expression of this receptor in man.

Further provided by this invention is a method of determining the physiological effects of expressing varying levels of human or mammalian 5-HT₄ receptors which comprises producing a transgenic nonhuman animal whose levels of human or mammalian 5-HT₄ receptor expression are varied by use of an inducible promoter which regulates receptor expression. This invention also provides a method of determining the physiological effects of expressing varying levels of human or mammalian 5-HT₄ receptor which comprises producing a panel of transgenic nonhuman animals each expressing a different amount of human or mammalian 5-HT₄ receptor. Such animals may be produced by introducing different amounts of DNA encoding a human or mammalian 5-HT₄ receptor into the oocytes from which the transgenic animals are developed.

This invention also provides a method for identifying a substance capable of alleviating abnormalities resulting from overexpression of a human or mammalian 5-HT₄ receptor comprising administering the substance to a transgenic nonhuman mammal expressing at least one artificially introduced DNA molecule encoding a human or mammalian 5-HT₄ receptor and determining whether the substance alleviates the physical and behavioral abnormalities displayed by the transgenic nonhuman mammal as a result of overexpression of a human or mammalian 5-HT₄ receptor. As used herein, the term “substance” means a compound or composition which may be natural, synthetic, or a product derived from screening. Examples of DNA molecules are DNA or cDNA molecules having a coding sequence substantially the same as the coding sequences shown in FIGS. 1, 2, and 5 (SEQ ID NOs. 1, 3, and 5).

This invention provides a pharmaceutical composition comprising an amount of the substance described supra effective to alleviate the abnormalities resulting from overexpression of 5-HT₄ receptor and a pharmaceutically acceptable carrier.

This invention further provides a method for treating the abnormalities resulting from overexpression of a human or mammalian 5-HT₄ receptor which comprises administering to a subject an amount of the pharmaceutical composition described above effective to alleviate the abnormalities resulting from overexpression of a human or mammalian 5-HT₄ receptor.

This invention provides a method for identifying a substance capable of alleviating the abnormalities resulting from underexpression of a human or mammalian 5-HT₄ receptor comprising administering the substance to the transgenic nonhuman mammal described above which expresses only nonfunctional human or mammalian 5-HT₄ receptor and determining whether the substance alleviates the physical and behavioral abnormalities displayed by the transgenic nonhuman mammal as a result of underexpression of a human or mammalian 5-HT₄ receptor.

This invention also provides a pharmaceutical composition comprising an amount of a substance effective to alleviate abnormalities resulting from underexpression of a human or mammalian 5-HT₄ receptor and a pharmaceutically acceptable carrier.

This invention further provides a method for treating the abnormalities resulting from underexpression of a human or mammalian 5-HT₄ receptor which comprises administering to a subject an amount of the pharmaceutical composition described above effective to alleviate the abnormalities resulting from underexpression of a human or mammalian 5-HT₄ receptor.

This invention provides a method for diagnosing a predisposition to a disorder associated with the expression of a human or mammalian 5-HT₄ receptor allele which comprises: a) obtaining DNA of subjects suffering from the disorder; b) performing a restriction digest of the DNA with a panel of restriction enzymes; c) electrophoretically separating the resulting DNA fragments on a sizing gel; d) contacting the resulting gel with a nucleic acid probe capable of specifically hybridizing to DNA encoding a human or mammalian 5-HT₄ receptor and labelled with a detectable marker; e) detecting labelled bands which have hybridized to the DNA encoding a human or mammalian 5-HT₄ receptor labelled with a detectable marker to create a unique band pattern specific to the DNA of subjects suffering from the disorder; f) preparing DNA obtained for diagnosis by steps a-e; and g) comparing the unique band pattern specific to the DNA of subjects suffering from the disorder from step e and the DNA obtained for diagnosis from step f to determine whether the patterns are the same or different and thereby to diagnose predisposition to the disorder if the patterns are the same. This method may also be used to diagnose a disorder associated with the expression of a specific human 5-HT₄ receptor allele or mammalian 5-HT₄ receptor allele.

This invention provides a method of preparing the isolated 5-HT₄ receptor which comprises inducing cells to express receptor, recovering the receptor from the resulting cells, and purifying the receptor so recovered. An example of a 5-HT₄ receptor is an isolated protein having substantially the same amino acid sequence as the amino acid sequence shown in FIG. 5. For example, cells can be induced to express receptors by exposure to substances such as hormones. The cells can then be homogenized and the receptor isolated from the homogenate using an affinity column comprising, for example serotonin or another substance which is known to bind to the 5-HT₄ receptor. The resulting fractions can then be purified by contacting them with an ion exchange column, and determining which fraction contains 5-HT₄ receptor activity or binds anti-receptor antibodies.

This invention provides a method of preparing an isolated human 5-HT₄ receptor which comprises inserting nucleic acid encoding the human 5-HT₄ receptor in a suitable vector, inserting the resulting vector in a suitable host cell, recovering the receptor produced by the resulting cell, and purifying the receptor so recovered. An example of an isolated human 5-HT₄ receptor is an isolated protein having substantially the same amino acid sequence as the amino acid sequence shown in FIG. 5 (SEQ ID NOs. 5 and 6). This invention provides a method of preparing an isolated mammalian 5-HT₄ receptor which comprises inserting nucleic acid encoding the mammalian 5-HT₄ receptor in a suitable vector, inserting the resulting vector in a suitable host cell, recovering the receptor produced by the resulting cell, and purifying the receptor so recovered. An example of an isolated mammalian 5-HT₄ receptor is an isolated protein having substantially the same amino acid sequence as the amino acid sequence shown in FIGS. 1 and 2 (SEQ ID NOs. 1-2 and Seq I. D. Nos. 3-4, respectively). These methods for preparing 5-HT₄ receptor uses recombinant DNA technology methods well known in the art. For example, isolated nucleic acid encoding 5-HT₄ receptor is inserted in a suitable vector, such as an expression vector. A suitable host cell, such as a bacterial cell, insect cell, or a eukaryotic cell such as a yeast cell, is transfected with the vector. 5-HT₄ receptor is isolated from the culture medium by affinity purification or by chromatography or other methods well known in the art.

This invention provides a method for determining whether a compound not known to be capable of specifically binding to a human 5-HT₄ receptor can specifically bind to the human 5-HT₄ receptor, which comprises contacting a mammalian cell comprising a plasmid adapted for expression in a mammalian cell which plasmid further comprises a DNA which expresses a human 5-HT₄ receptor on the cell's surface with the compound under conditions permitting binding of ligands known to bind to a human 5-HT₄ receptor, detecting the presence of any compound bound to the human 5-HT₄ receptor, the presence of bound compound indicating that the compound is capable of specifically binding to the human 5-HT₄ receptor.

This invention provides a method for determining whether a compound not known to be capable of specifically binding to a mammalian 5-HT₄ receptor can specifically bind to the mammalian 5-HT₄ receptor, which comprises contacting a mammalian cell comprising a plasmid adapted for expression in a mammalian cell which plasmid further comprises a DNA which expresses a mammalian 5-HT₄ receptor on the cell's surface with the compound under conditions permitting binding of ligands known to bind to a mammalian 5-HT₄ receptor, detecting the presence of any compound bound to the human 5-HT₄ receptor, the presence of bound compound indicating that the compound is capable of specifically binding to the mammalian 5-HT₄ receptor.

This invention provides a method for identifying a compound which is not known to be capable of binding to a human 5-HT₄ receptor can functionally activate the human 5-HT₄ receptor on the surface of a mammalian cell or prevent a ligand which does so, which comprises contacting the mammalian cell which cell comprises a plasmid adapted for expression in the mammalian cell such plasmid further comprising DNA which expresses the human 5-HT₄ receptor on the surface of the mammalian cell with the compound, determining whether the compound activates the human 5-HT₄ receptor or prevents a ligand which does so, and thereby identifying the compound as a compound which is binds to and functionally activates the human 5-HT₄ receptor or prevents the functional activation of the human 5-HT₄ receptor by a ligand which does so. The DNA in the cell may have a coding sequence substantially the same as the coding sequence shown in FIG. 5 ( SEQ ID No. 5).

This invention provides a method for identifying a compound which is not known to be capable of binding to a mammalian 5-HT₄ receptor can functionally activate the mammalian 5-HT₄ receptor on the surface of a mammalian cell or prevent a ligand which does so, which comprises contacting the mammalian cell which cell comprises a plasmid adapted for expression in the mammalian cell such plasmid further comprising DNA which expresses the mammalian 5-HT₄ receptor on the surface of the mammalian cell with the compound, determining whether the compound activates the mammalian 5-HT₄ receptor or prevents a ligand which does so, and thereby identifying the compound as a compound which is binds to and functionally activates the mammalian 5-HT₄ receptor or prevents the functional activation of the mammalian 5-HT₄ receptor by a ligand which does so. The DNA in the cell may have a coding sequence substantially the same as the coding sequence shown in FIGS. 1 and 2 (SEQ ID NOs. 1 and 3).

The activation or blockade of the functional response is detected by means of a bioassay from the mammalian cell such as a second messenger response, and thereby determining whether the compound activates or prevents the activation of the 5-HT₄ receptor functional output. Preferably, the mammalian cell is nonneuronal in origin. An example of a nonneuronal mammalian cell is an LM (tk−) cell. Another example of a non-neuronal mammalian cell to be used for functional assays is a murine fibroblast cell line, specifically the NIH3T3 cell. The preferred method for determining whether a compound is capable of binding to the 5-HT₄ receptor comprises contacting a transfected nonneuronal mammalian cell (i.e. a cell that does not naturally express any type of 5-HT or G-protein coupled receptor, thus will only express such a receptor if it is transfected into the cell) expressing a 5-HT₄ receptor on its surface, or contacting a membrane preparation derived from such a transfected cell, with the compound under conditions which are known to prevail, and thus to be associated with, in vivo binding of ligands to a 5-HT₄ receptor, detecting the presence of any of the compound being tested bound to the 5-HT₄ receptor on the surface of the cell, and thereby determining whether the compound binds to, and activates or prevents the activation of the 5-HT₄ receptor. This response system is obtained by transfection of isolated DNA into a suitable host cell containing the desired second messenger system such as phosphoinositide hydrolysis, adenylate cyclase, guanylate cyclase or ion channels. Such a host system is isolated from pre-existing cell lines, or can be generated by inserting appropriate components of second messenger systems into existing cell lines. Such a transfection system provides a complete response system for investigation or assay of the activity of human 5-HT₄ receptor with compounds as described above.

Transfection systems are useful as living cell cultures for competitive binding assays between known or candidate drugs and ligands which bind to the receptor and which are labeled by radioactive, spectroscopic or other reagents. Membrane preparations containing the receptor isolated from transfected cells are also useful for these competitive binding assays. Functional assays of second messenger systems or their sequelae in transfection systems act as assays for binding affinity and efficacy in the activation of receptor function. A transfection system constitutes a “drug discovery system” useful for the identification of natural or synthetic compounds with potential for drug development that can be further modified or used directly as therapeutic compounds to activate or inhibit the natural functions of the human 5-HT₄ receptor. The transfection system is also useful for determining the affinity and efficacy of known drugs at human 5-HT₄ receptor sites.

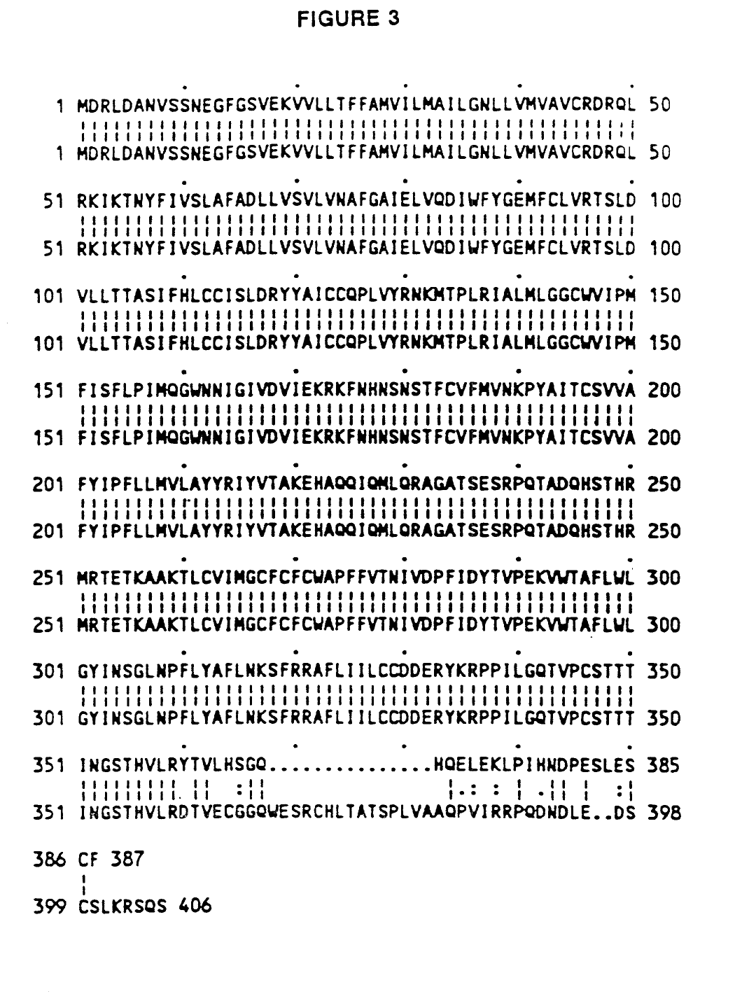

This invention also provides a method of screening compounds to identify drugs which interact with, and specifically bind to, a human 5-HT₄ receptor on the surface of a cell, which comprises contacting a mammalian cell which comprises a plasmid adapted for expression in a mammalian cell which plasmid further comprises DNA which expresses a human 5-HT₄ receptor on the cell's surface with a plurality of compounds, determining those compounds which bind to the human 5-HT₄ receptor expressed on the cell surface of the mammalian cell, and thereby identifying compounds which interact with, and specifically bind to, the human 5-HT₄ receptor. The DNA in the cell may have a coding sequence substantially the same as the coding sequence shown in FIG. 5 (SEQ ID NO. 5). This invention also provides a method of screening compounds to identify drugs which interact with, and specifically bind to, a mammalian 5-HT₄ receptor on the surface of a cell, which comprises contacting a mammalian cell which comprises a plasmid adapted for expression in a mammalian cell which plasmid further comprises DNA which expresses a mammalian 5-HT₄ receptor on the cell's surface with a plurality of compounds, determining those compounds which bind to the mammalian 5-HT₄ receptor expressed on the cell surface of the mammalian cell, and thereby identifying compounds which interact with, and specifically bind to, the mammalian 5-HT₄ receptor. The DNA in the cell may have a coding sequence substantially the same as the coding sequence shown in FIGS. 1 and 3 (SEQ ID NOs. 1 and 2). Various methods of detection may be employed. The compounds may be “labeled” by association with a detectable marker substance (e.g., radiolabel or a non-isotopic label such as biotin). Preferably, the mammalian cell is nonneuronal in origin. An example of a nonneuronal mammalian cell is a Cos-7 cell. Drug candidates are identified by choosing chemical compounds which bind with high affinity to the expressed 5-HT₄ receptor protein in transfected cells, using radioligand binding methods well known in the art, examples of which are shown in the binding assays described herein. Drug candidates are also screened for selectivity by identifying compounds which bind with high affinity to one particular receptor but do not bind with high affinity to any other receptor subtypes or to any other known receptor. Because selective, high affinity compounds interact primarily with the target 5-HT₄ receptor site after administration to the patient, the chances of producing a drug with unwanted side effects are minimized by this approach.

This invention provides a pharmaceutical composition comprising a drug identified by the method described above and a pharmaceutically acceptable carrier. As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. Once the candidate drug has been shown to be adequately bioavailable following a particular route of administration, for example orally or by injection (adequate therapeutic concentrations must be maintained at the site of action for an adequate period to gain the desired therapeutic benefit), and has been shown to be non-toxic and therapeutically effective in appropriate disease models, the drug may be administered to patients by that route of administration determined to make the drug bioavailable, in an appropriate solid or solution formulation, to gain the desired therapeutic benefit.

Applicants have identified a novel 5-HT₄ receptor subtype protein, designated 5-HT₄ and have described methods for the identification of pharmacological compounds for therapeutic treatments. Pharmacological compounds which are directed against specific receptor subtypes provide effective new therapies with minimal side effects.

Elucidation of the molecular structures of the neuronal serotonin receptors is an important step in the understanding of serotonergic neurotransmission. This disclosure reports the isolation and amino acid sequence of a novel cDNA which encodes a human 5-HT₄ receptor. This disclosure reports the isolation, amino acid sequence, and functional expression of a two novel cDNAs which encode mammalian 5-HT₄ receptors. The identification of 5-HT₄ receptor subtypes play a pivotal role in elucidating the molecular mechanisms underlying serotonergic transmission, and should also aid in the development of novel therapeutic agents.

A complementary DNA clone (designated pBluescript-hS10) encoding a serotonin receptor subtype, 5-HT₄, has been isolated from human brain, human heart and human retina. Additionally, two complementary DNA clones encoding the serotonin 5-HT₄ receptor subtype have been isolated from mammalian brain and their functional properties have been examined in mammalian cells. Analysis of 5-HT₄ structure and function provides a model for the development of drugs useful for the treatment of gastrointestinal conditions including irritable bowel disease, postoperative ileus, diabetic gastroparesis, emesis, achalasia, hiatal hernia, and esophageal spasm. In addition, 5-HT₄ receptors have been described functionally in the heart (Kaumann, 1992), adrenal (Lefebvre et al., 1992), and bladder (Corsi et al., 1991) indicating possible roles in cardiac rate and force of contraction, endocrine control of cortisol secretion, and urinary incontinence or spasticity. 5-HT₄ receptors have also been described in the brain, particularly in areas such as the hippocampus, in which we have localized the gene encoding 5-HT₄ receptors (S10-95), indicating a potential role in cognitive enhancement (Bockaert et al., 1992).

This invention identifies a mammalian serotonin receptor, its amino acid sequence, and its mammalian gene, the activation of which is coupled to activation of adenylate cyclase. The information and experimental tools provided by this discovery are useful to generate new therapeutic agents, and new therapeutic or diagnostic assays for this receptor protein, its associated mRNA molecule or its associated genomic DNA. The information and experimental tools provided by this discovery will be useful to generate new therapeutic agents, and new therapeutic or diagnostic assays for this new serotonin receptor subtype, its associated mRNA molecule, or its associated genoric DNA.

Specifically, this invention relates to the isolation of human cDNA clone and mammalian cDNA clones encoding a new serotonin receptor, designated 5-HT₄. In addition, the mammalian 5-HT₄ receptors have been expressed in COS-7 cells by transfecting the cells with the plasmids pcEXV-S10-87 and pcEXV-S10-95. The pharmacological binding properties of the encoded 5-HT₄ receptor have been determined, and the binding properties classify this receptor as a novel serotonin receptor. Mammalian cell lines expressing the mammalian 5-HT₄ receptor on the cell surface have been constructed, thus establishing the first well-defined, cultured cell lines with which to study the novel 5-HT₄ receptor.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Methods and Materials

PCR Amplification: The third (III) and fifth (V) transmembrane (TM) domains of the following receptors were aligned and used to synthesize a pair of degenerate primers: 5-HT_{1A}, 5-HT_{1C}, 5-HT₂ and the 5-HT_{1Dα/β} receptors. Primers 3.17 and 5.5 ([5′-TGGAATTCTG(C/T)G(C/T)IAT(A/C/T)(G/T)CICTGGA(C/T)(A/C) G(C/G)TA-3′] (SEQ ID No. 9), [5′-CATIA(G/C/A)I(G/A)IIA(G/A)IGG(T/G/A/)AT(G/A)(T/A)A(G/A)AAIGC-3′]) (SEQ ID No. 10) were used to amplify 5 μg of poly (A+) RNA from rat brain that was reverse transcribed by avian myeloblastosis virus reverse transcriptase (AMV). PCR was performed on single-stranded cDNA under the following conditions: 94° C. for 1 min, 50° C. for 2 min and 72° C. for 3 min for 40 cycles. Following PCR, 90 μl of the reaction was phenol:chloroform extracted and precipitated; 10μl was visualized on a gel using ethidium bromide staining. After precipitation the sample was treated with T₄ DNA polymerase and digested with EcoR1 prior to separation on a 1% agarose gel. The DNA fragments (200 to 400 base pairs) were isolated from the gel, kinased and cloned into pBluescript. Recombinant clones were analyzed by sequencing. One fragment 270 base pairs in length, named S10, was identified. This sequence contained a “TM IV” like domain and represented a potentially new serotonin receptor. The corresponding full length cDNA was isolated from a rat brain cDNA library.

Rat PCR primers (from TM3 to TM7) were used to amplify single-stranded cDNA prepared from human heart, brain and retina, as described above. Those human PCR DNA fragments were subcloned in pBluescript and sequenced.

cDNA Library Construction, screening and Sequencing: Rat brains were dissected from adult male CD rats (Charles River Laboratories) and total RNA was prepared by the guanidine thiocyanate method (Chirgwin, J. W. et al.: 1979). Poly A⁺RNA was purified with a Fast track kit (Invitrogen Corp., San Diego, Calif.). Double stranded (DS) cDNA was synthesized from 5 μg of poly A⁺RNA according to Gubler and Hoffman (Gubler, U. and B. J. Hoffman, 1983). The resulting DS cDNA was ligated to BstxI/EcoRI adaptors (Invitrogen Corp.), the excess of adaptors was removed by chromatography on Sepharose CL 4B (Pharmacia LKB) and the DNA was then size selected on a Gen-Pak Fax HPLC column (Zhao, D. et al., 1992) (Waters, Millipore Corp., Milford, Mass.). High molecular weight fractions were ligated in pCDM8 cut by BstxI (Invitrogen Corp.). The ligated DNA was electroporated in E.Coli MC 1061 (Gene Pulser, Biorad). A total of 20×10⁶ independent clones with an insert mean size of 1.9 kb could be generated. Before amplification, the library was divided into pools of 2.5 to 5×10⁴ independent clones. After 18 hours amplification, the pools were stored at −85° C. in 20% glycerol.

100 pools of the cDNA library, representing 3.2×10⁶ primary clones, were screened using exact PCR primers derived from the S10 PCR clone sequence. 1 μl (4×10⁶ bacteria) of each amplified pool was subjected directly to 40 cycles of PCR and the resulting products analyzed by agarose gel electrophoresis and Southern blotting. Two out of four positive pools were analyzed further and by sib selection and plating out, two individual full length cDNA clones, S10-87 and S10-95, were isolated. DS-DNA was sequenced with a sequanase kit (US Biochemical, Cleveland, Ohio) according to the manufacturer. Nucleotide and peptide sequences analysis were performed with GCG programs.

Genomic Cloning and Sequencing: A human fibroblast genomic library in λ dash II (≈1.5×10⁶ total recombinants; Stratagene, LaJolla, Calif.) was screened using a 45 nt. oligonucleotide probe derived from the rat S10-87 receptor gene, designed in the 3′ end of the carboxyl terminal tail (from the anti-sense strand [nucleotide 1220-1264), 5′ TCAAAAGCATGATTCCAGGGACTCTGGGTCATTGTGTATGGGCAA 3′ (SEQ ID No. 11) (see FIG. 1). The oligomer was labeled with [³²P]γATP by using polynucleotide kinase. Hybridization was performed at medium stringency conditions: 45° C. in a solution containing 37.5% formamide, 5×SSC (1×SSC is 0.15M sodium chloride, 0.015M sodium citrate), 1×Denhardt's solution (0.02% polyvinylpyrrolindone, 0.02% Ficoll, 0.02% bovine serum albumin), and 200 μg/μl sonicated salmon sperm DNA. The filters were washed at 45° C. in 0.1×SSC containing 0.1% sodium dodecyl sulfate and exposed at −70° C. to Kodak XAR film in the presence of an intensifying screen. Lambda phage clones hybridizing with the probe were plaque purified and DNA was prepared for Southern blot analysis (Southern, 1975; Sambrook et al., 1989). A 900 bp Hind2/SstI hybridizing fragment was subcloned into pUC18 (Pharmacia, Piscataway, N.J.)). Nucleotide sequence analysis was accomplished by the Sanger dideoxy nucleotide chain termination method (Sanger et al., 1977) on denatured double-stranded plasmid templates, using Sequenase (US Biochemical Corp., Cleveland, Ohio).

PCR amplification of a partial length human 810-87 cDNA clone:

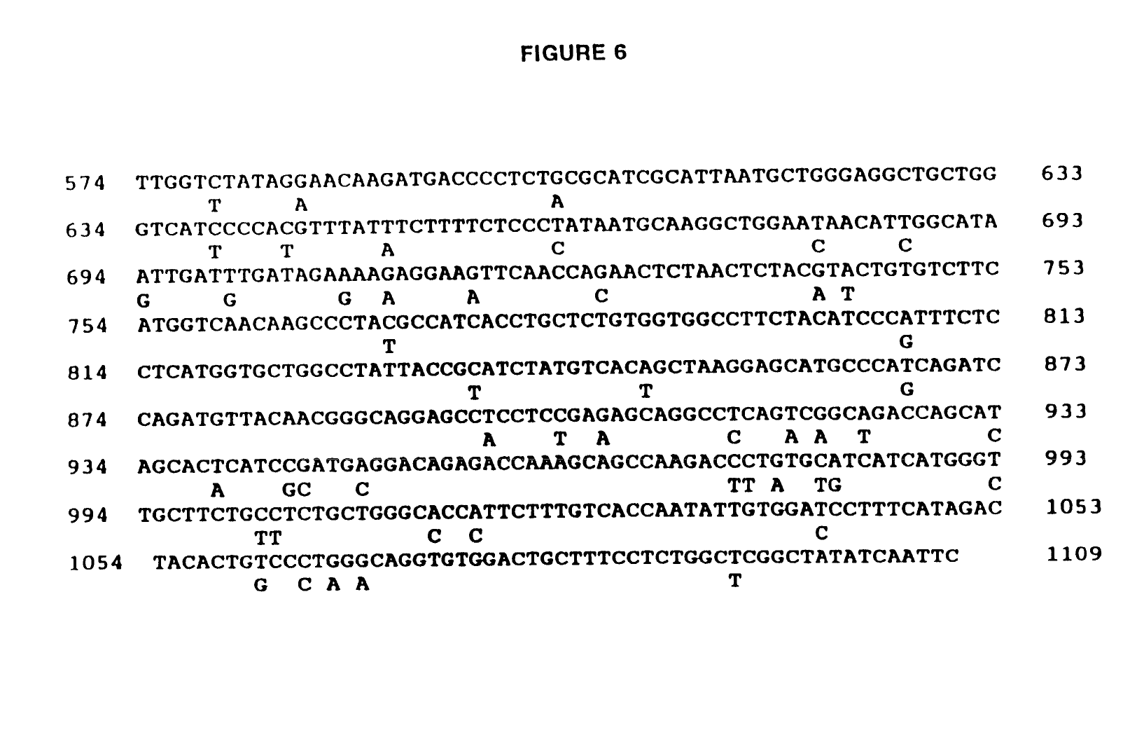

The 900 bp Hind2/SstI fragment contained sequence encoding the human S10-87 carboxy terminal tail, including the stop codon. This sequence was used to generate a 25 mer (reverse primer) containing the stop codon: 5′ CCTCAATCAGAAGCATGATTCCAGG 3′ (SEQ ID No. 12). As a forward primer we used the 5′ end of the human PCR fragment previously identified (FIG. 6): 5′TTGGTCTATAGGAACAAGATGACCC 3′ (SEQ ID No. 13). These human PCR primers were used to amplify single stranded cDNA prepared from human brain as previously described. The amplified DNA was subcloned and sequenced as described above.

Isolation of the full length human S10-95 cDNA clone: 20 pools of a human hippocampal cDNA library (3 kb average size insert, in pcEXV-3) representing 10⁶ independent clones were screened by PCR with TM4-TM6 primers as previously described. Five positive pools were identified. One of those pools was analyzed further and by sib selection a 5 kb cDNA clone, CG-17, was isolated. Double Stranded-DNA was sequenced as described above. Nucleotide and peptide sequence analysis were performed with the Genetics Computer Group sequence analysis software package.

DNA transfection: The full coding region of S10-87 (clone CG-5) and S10-95 (clones CG-6 and CG-17) were subcloned in the correct orientation in the mammalian expression vectors pCDNA1-Amp (Invitrogen Corp.), and pcEXV-3 (Miller, J. and R. N. Germain, 1986) (CG-7 and CG-8 respectively). For transient expression, Cos-7 cells were transfected by the DEAE-Dextran method, using 1 μg of DNA /10⁶ cells (Warden, D. and H. V. Thorne, 1968).

Membrane Preparation: Membranes were prepared from transiently transfected COS-7 cells which were grown to 100% confluence. The cells were washed twice with phosphate-buffered saline, scraped from the culture dishes into 5 ml of ice-cold phosphate-buffered saline, and centrifuged at 200×g for 5 min at 4′. The pellet was resuspended in 2.5 ml of ice-cold Tris buffer (20 mM Tris -HCl, pH 7.4 at 23°, 5 mM EDTA), and homogenized by a Wheaton tissue grinder. The lysate was subsequently centrifuged at 200×g for 5 min at 4° to pellet large fragments which were discarded. The supernatant was collected and centrifuged at 40,000×g for 20 min at 4°. The pellet resulting from this centrifugation was washed once in ice-cold Tris wash buffer and finally resuspended in a final buffer containing 50 mM Tris-HCl and 0.5 mM EDTA, pH 7.4 at 23°. Membrane preparations were kept on ice and utilized within two hr for the radioligand binding assays. Protein concentrations were determined by the method of Bradford (1976) using bovine serum albumin as the standard.

Radioligand Binding: [²H]5-HT binding was performed using slight modifications of the 5-HT_{1D} assay conditions reported by Herrick-Davis and Titeler (1989) with the omission of masking ligands. Radioligand binding studies were achieved at 37° C. in a total volume of 250 μl of buffer (50 mM Tris, 10 nM MgCl₂, 0.2 mM EDTA, 10 μM pargyline, 0.1% ascorbate, pH 7.4 at 37° C.) in 96 well microtiter plates. Saturation studies were conducted using [³H]5-HT at 10 different concentrations ranging from 1.0 nM to 100 nM. Displacement studies were performed using 10 nM [³H]5-HT. The binding profile of drugs in competition experiments was established using 7 concentrations of compound. Incubation times were 30 min for both saturation and displacement studies. Nonspecific binding was defined in the presence of 10 μM 5-HT. Binding was initiated by the addition of 50 μl membrane homogenates (10-20 μg). The reaction was terminated by rapid filtration through presoaked (0.5% polyethyleneimine) filters using 48R Cell Brandel Harvester (Gaithersburg, Md.). Subsequently, filters were washed for 5 sec with ice cold buffer (50 mM Tris HCL, pH 7.4 at 4° C.), dried and placed into vials containing 2.5 ml of Readi-Safe (Beckman, Fullerton, Calif.), and radioactivity was measured using a Beckman LS 6500C liquid scintillation counter. The efficiency of counting of [³H]5-HT averaged between 45-50%. Binding data were analyzed by computer-assisted nonlinear regression analysis (Accufit and Accucomp, Lundon Software, Chagrin Falls, Ohio). IC₅₀ values were converted to K_{f} values using the Cheng-Prusoff equation (1973).

[³H]GR113808 binding was performed using slight modifications of the method of Waeber et al., 1993. Radioligand binding studies were achieved at 37° C. in a total volume of 250 μl of buffer (50 mM Tris, 10 μM, 0.01% ascorbate, pH 7.4 at 37° C.) in 96 well microtiter plates. Saturation studies were conducted using [³H]GR113808 at 10-12 different concentrations ranging from 0.005-2.5 nM. Displacement studies were performed using 0.2-0.4 nM [³H]GR113808. The binding profile of drugs in competition experiments was established using 10-12 concentrations of compound. Incubation times were 30 min for both saturation and displacement studies. Nonspecific binding was defined in the presence of 50 μM 5-HT. Binding was initiated and terminated as described for [³H]5-HT binding (see above). Radioactivity was measured and data were analyzed as described above for [³H]5-HT.

Measurement of cAMP Formation: The transiently transfected Cos-7 cells were incubated in Dulbecco's modified Eagle's medium, 5 mM theophylline, 10 mM Hepes (4-[2-Hydroxyethyl]-1-piperazineethanesulfonic acid), 10 μM pargyline, and/or appropriate concentrations of antagonists for 20 minutes at 37° C., 5% CO₂. Serotonin or other agonists in the presence or absence of forskolin (FSK) (10 μM) were then added at appropriate concentrations and incubated for an additional 10 minutes at 37° C., 5% CO₂. The media was aspirated and the reaction stopped by the addition of 100 mM HCl. The plates were stored at 4° C. for 15 minutes, centrifuged for 5 minutes, 500×g to pellet cellular debris, and the supernatant aliquotted and stored at −20° C. prior to assessment of cAMP formation by radioimmunoassay (cAMP Radioimmunoassay kit, Advanced Magnetics, Cambridge, Mass.). Radioactivity was quantitated using a Packard COBRA Auto Gamma Counter equipped with data reduction software. Functional data was fitted to a four parameter logistic equation to obtain response parameters (EC₅₀, Eₘₐₓ, nH; Inplot, GraphPad, San Diego, Calif.).

Drugs: [³H]5-HT (specific activity=22.7 Ci/mmole) was obtained from New England Nuclear, Boston, Mass. [³H]GR113808 (specific activity=82 Ci/mmol) was obtained from Amersham International (Arlington Hts., Ill.). All other chemicals were obtained from commercial sources and were of the highest grade purity available.

Experimental Results

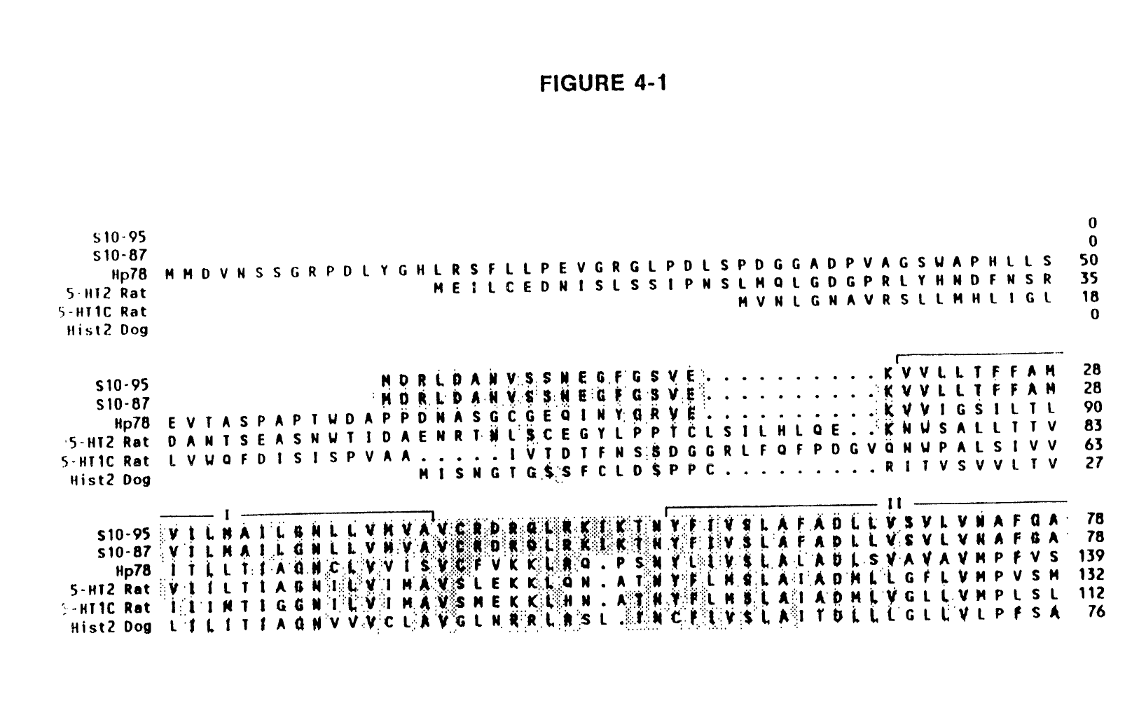

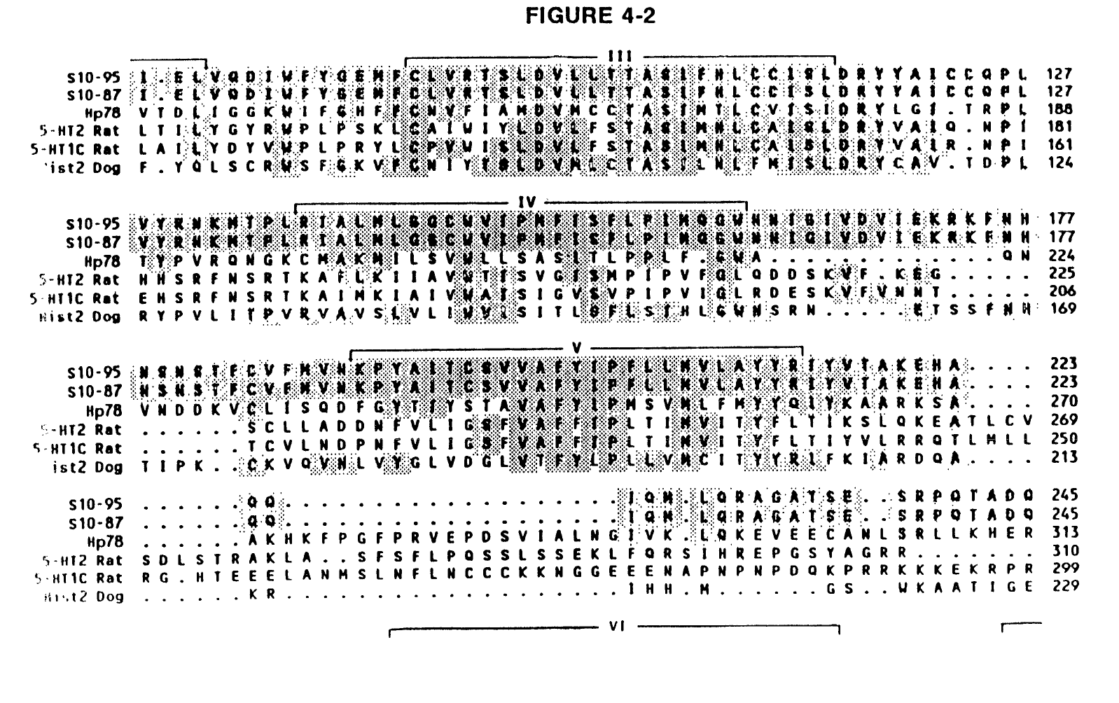

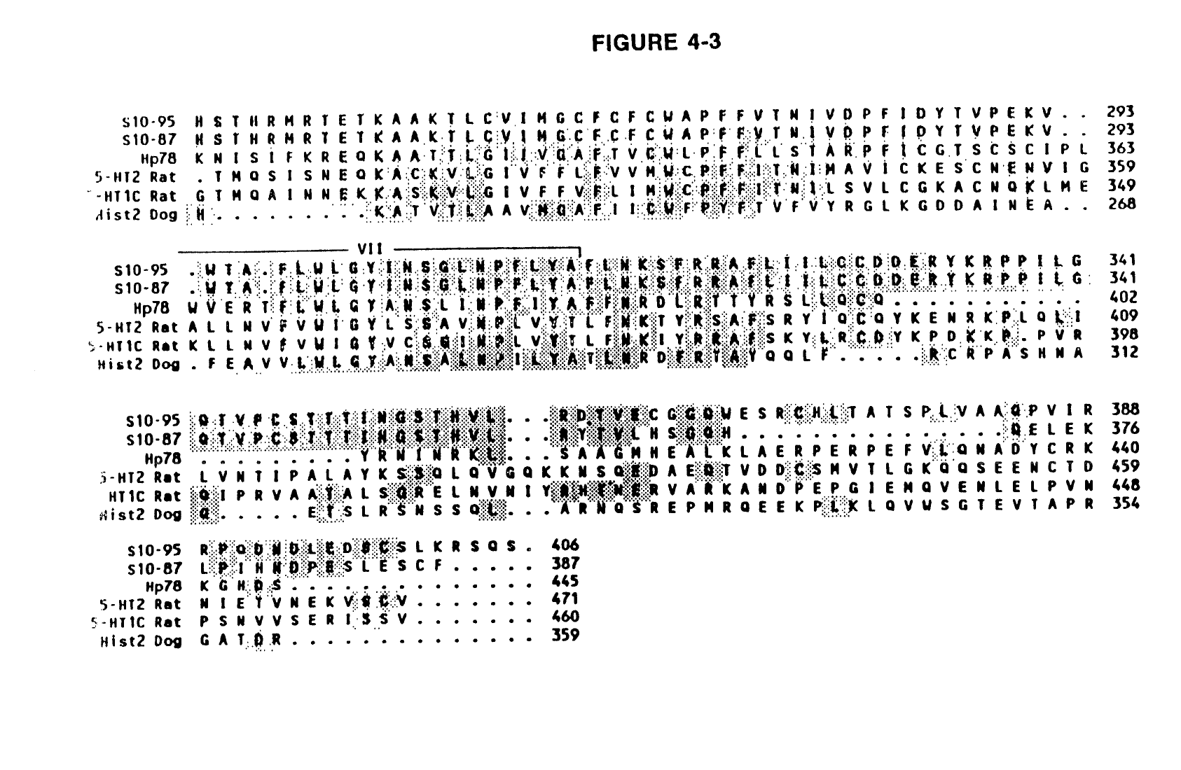

A 270 bp DNA fragment (S10) was identified when rat brain cDNA was used as template in a PCR amplification with two degenerate oligonucleotide primers derived from well conserved regions among several serotonin receptors, in the third and fifth putative transmembrane domains. The peptide sequence corresponding to this S10 PCR clone contained a “transmembrane IV like” domain. Since we used “serotonin receptor specific” PCR primers, this S10 clone represented a potentially new serotonin receptor. The corresponding full length cDNA was isolated from a rat brain cDNA library. Since five amplified commercial phage cDNA libraries turned out to be negative, we split the plasmid cDNA library into small pools of 2.5 to 5×10⁴ independent clones before amplification to avoid a potential growth bias against the S10 cDNA clone. By direct PCR analysis of bacterial pools, subsequent sib selection and standard filter hybridization, two cDNA clones were identified, S10-87 (5.5 kb) and S10-95 (4.5 kb). The nucleotide and deduced amino acid sequences are shown in FIG. 1 (S10-87) and FIG. 2 (S10-95). Surprisingly the peptide sequences between those two clones are only 96.7% identical, diverging in the second half of the carboxy terminus tails, downstream of position 359 (FIG. 3). In addition, the entire 3′ untranslated regions are totally divergent. The longest open reading frame for S10-87 encodes a protein of 387 amino acids and 406 amino acids for S10-95. The hydrophobicity plot displayed seven hydrophobic, putative membrane spanning regions which when compared to other G protein-coupled receptors did not show any significant high homologies, even to other serotonin receptors (Table 1 and FIG. 4). It is interesting to note that the highest homology, overall or restricted to the 7 TM region, is exhibited by the human histamine H₂ receptor, which like the 5-HT₄ receptor, is coupled to stimulation of cAMP.

Both S10-87 and S10-95 proteins carry 4 potential N-glycosylation sites in positions 7, 180, 316, and 352. They also possess 3 potential phosphorylation sites for protein kinase C in positions 218, 248, 318 and 4 potential phosphorylation sites for casein kinase II in positions 9, 97, 218 and 288. A potential palmitoylation site is present in both clones at the cysteine found in position 329. A large number of G protein-coupled receptors carry a cysteine in the same position and O'Dowd et al. have speculated that it plays an important role in the functional coupling of the human β₂-adrenergic receptor. In addition, clone S10-95 carries one more potential phosphorylation site for protein kinase C at position 400. This additional phosphorylation site could lead to differential functional coupling between the S10-87 and S10-95 receptors.

Since we isolated two different S10 cDNA clones by screening a library made from an entire brain, we checked for differential expression in seven different parts of the brain by PCR amplification using pairs of primers specific for each clone. The results are summarized in table 2. Clone S10-95 seems to be transcribed everywhere in the rat brain except in cerebellum. Clone S10-87 is only expressed in striatum. It remains to be determined if only one or both receptors are expressed in rat cortex.

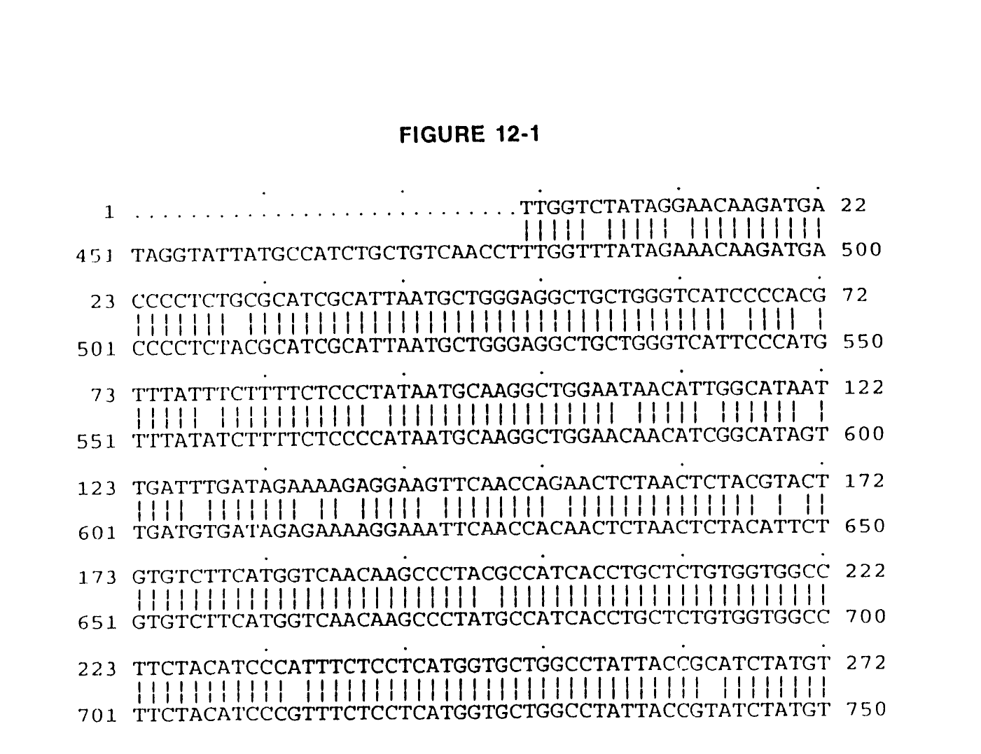

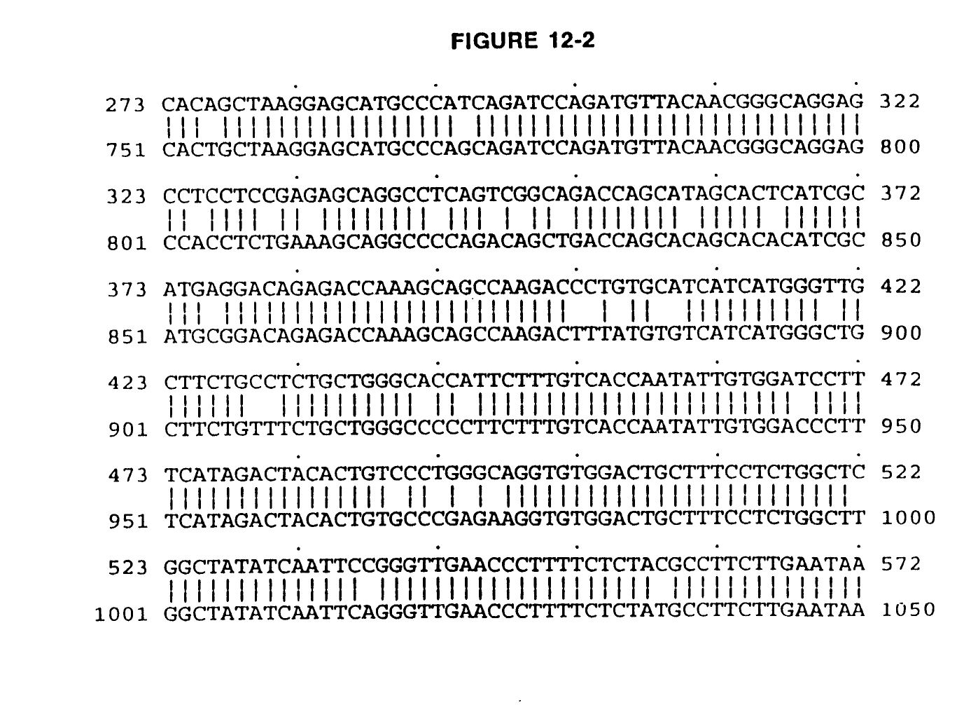

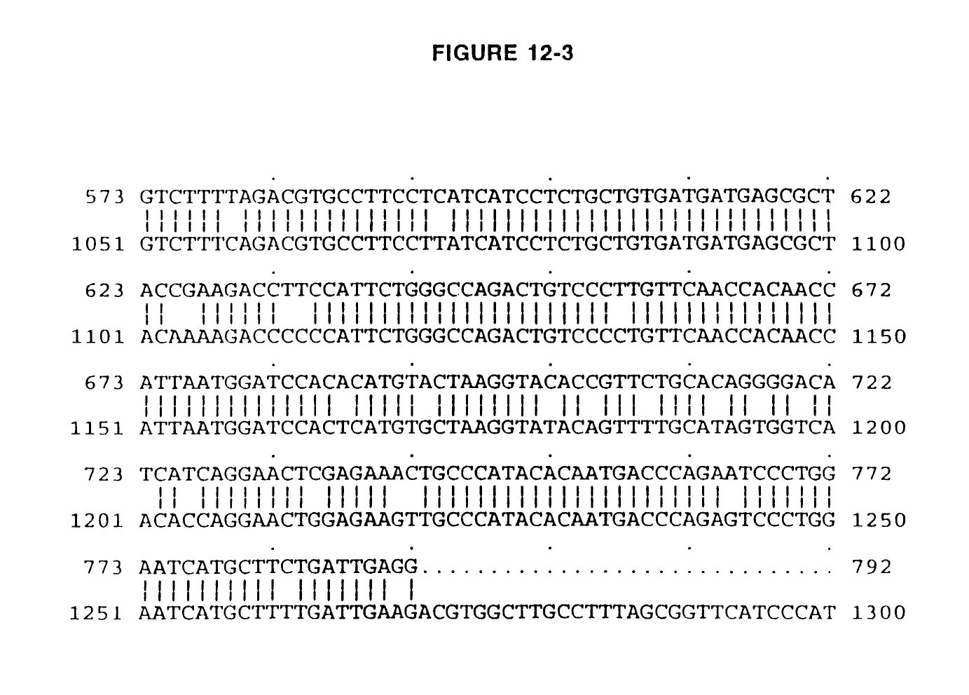

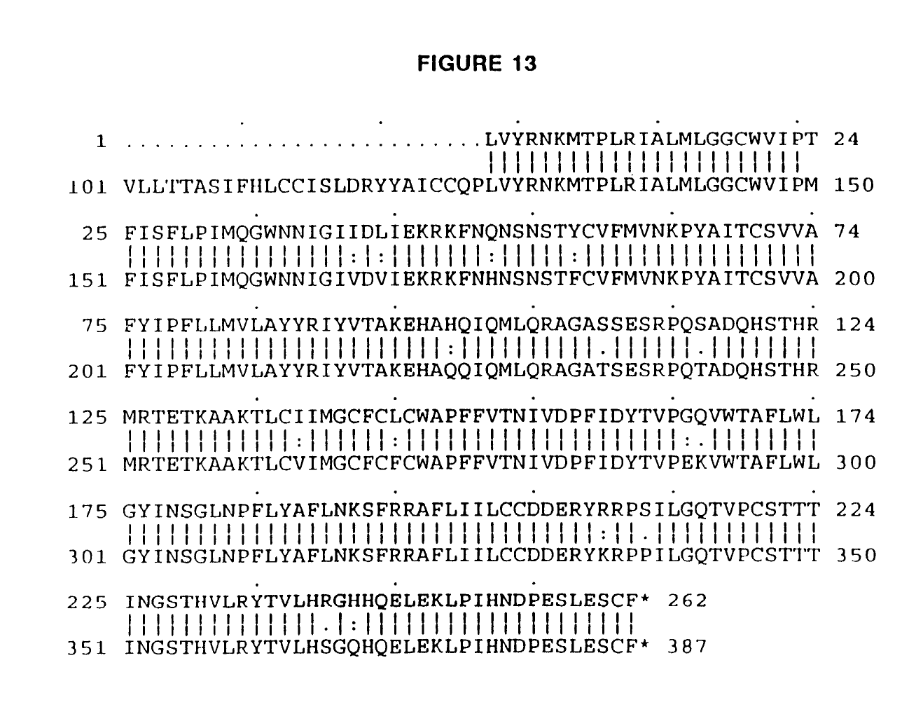

The partial human S10-87 nucleotide (FIG. 11A) and deduced amino acid sequences (FIG. 11B) are shown. The sequences are highly similar to the rat S10-87 homolog, 90.8% at the nucleotide level and 93.8% at the amino acid level (FIGS. 12 and 13 respectively).

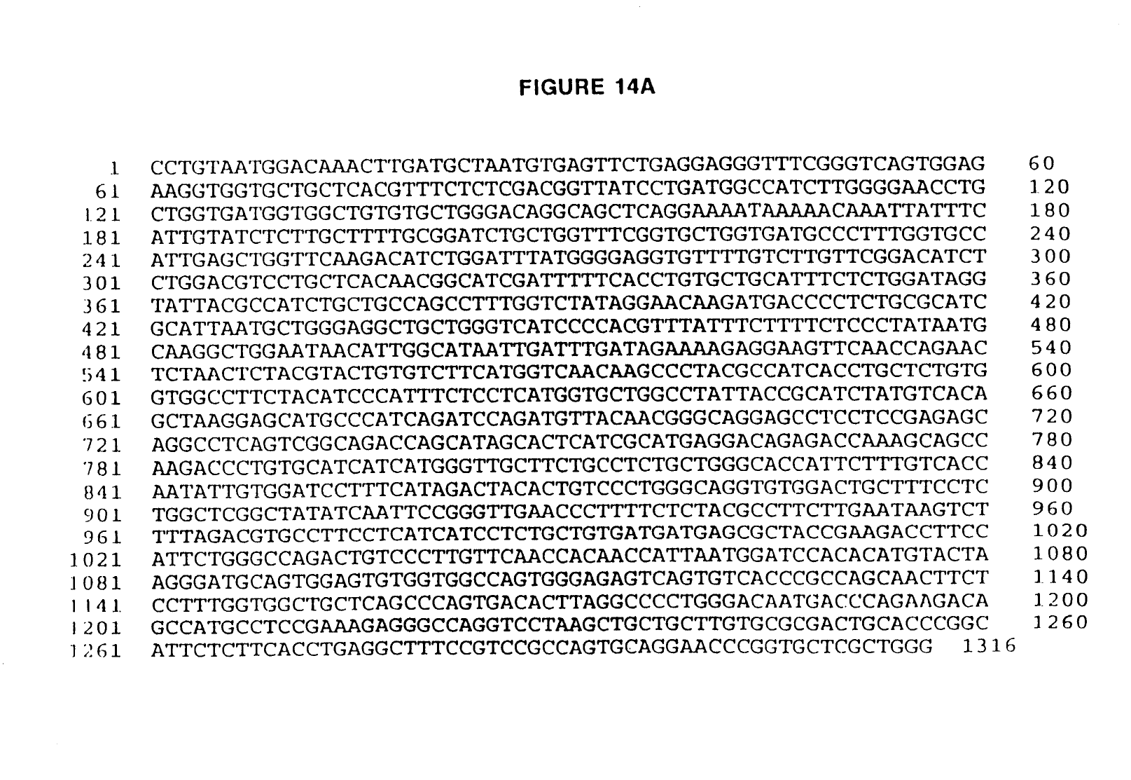

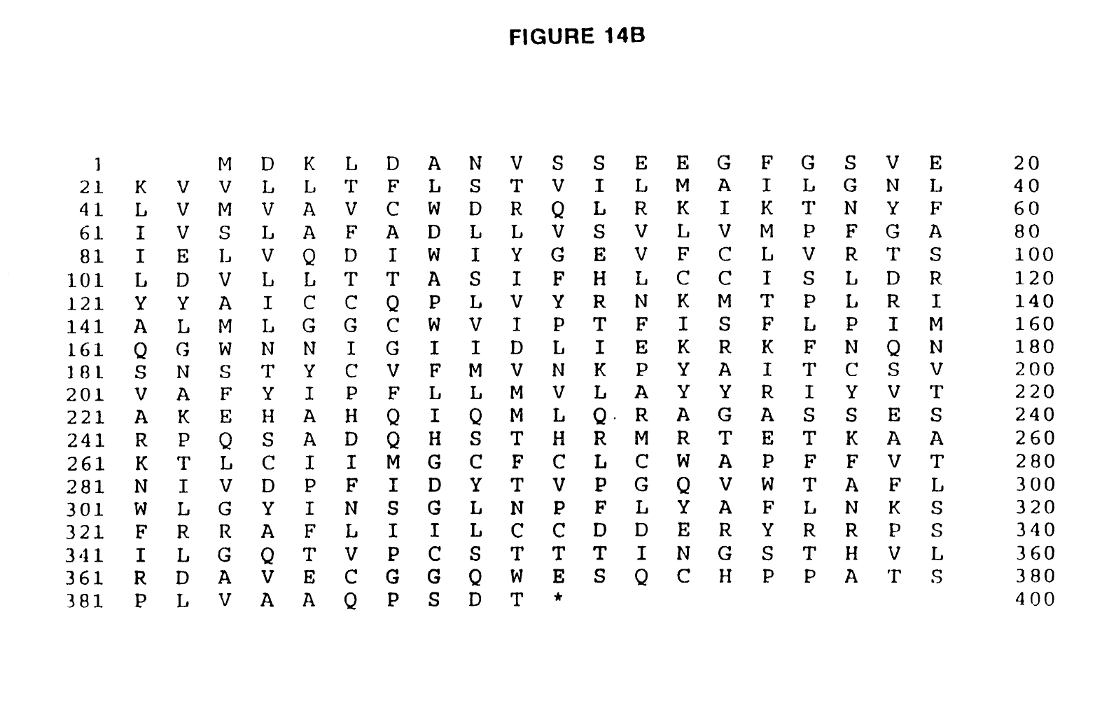

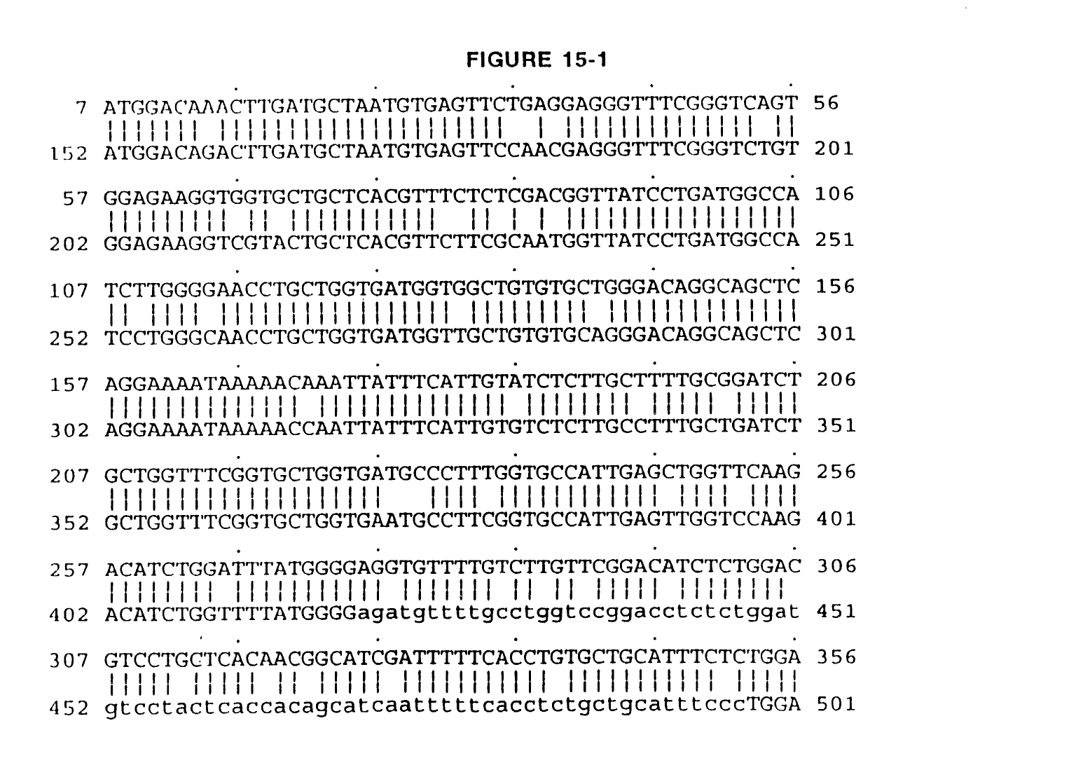

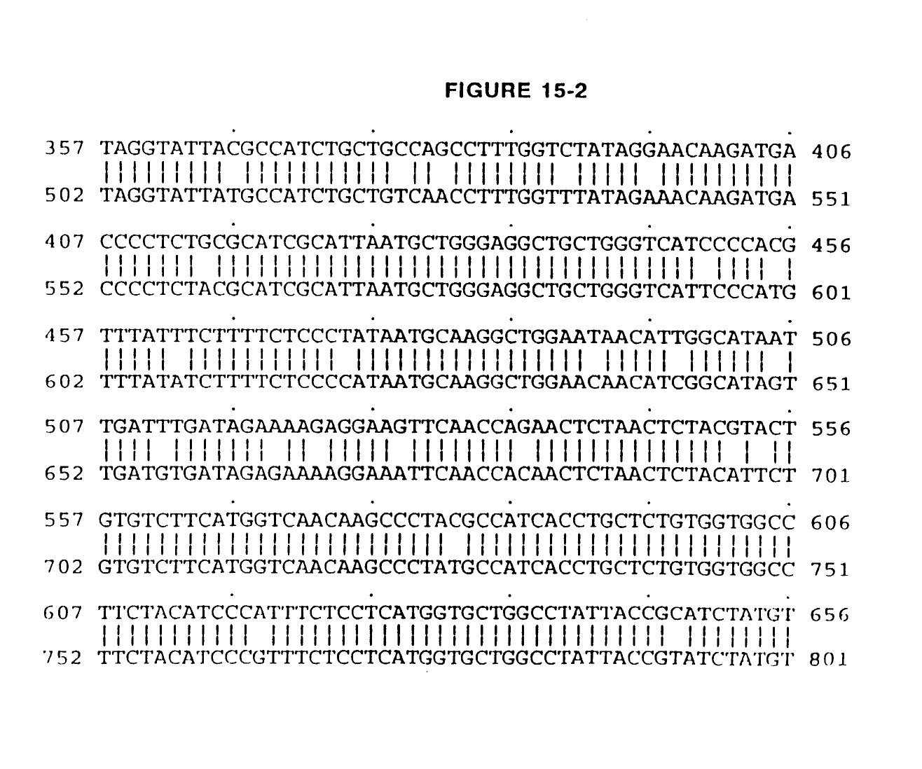

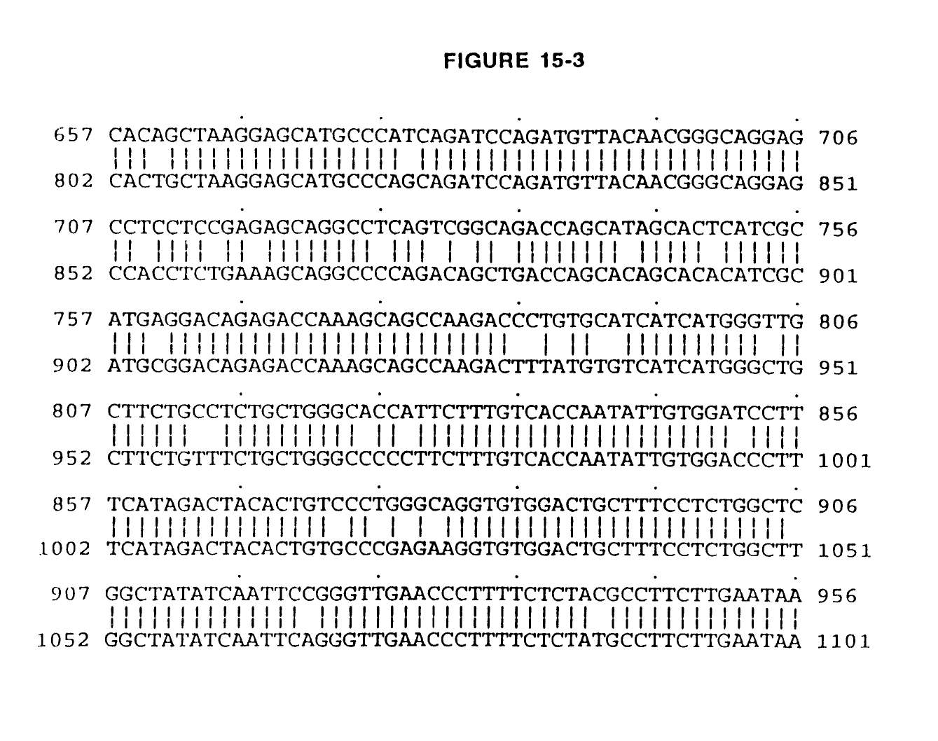

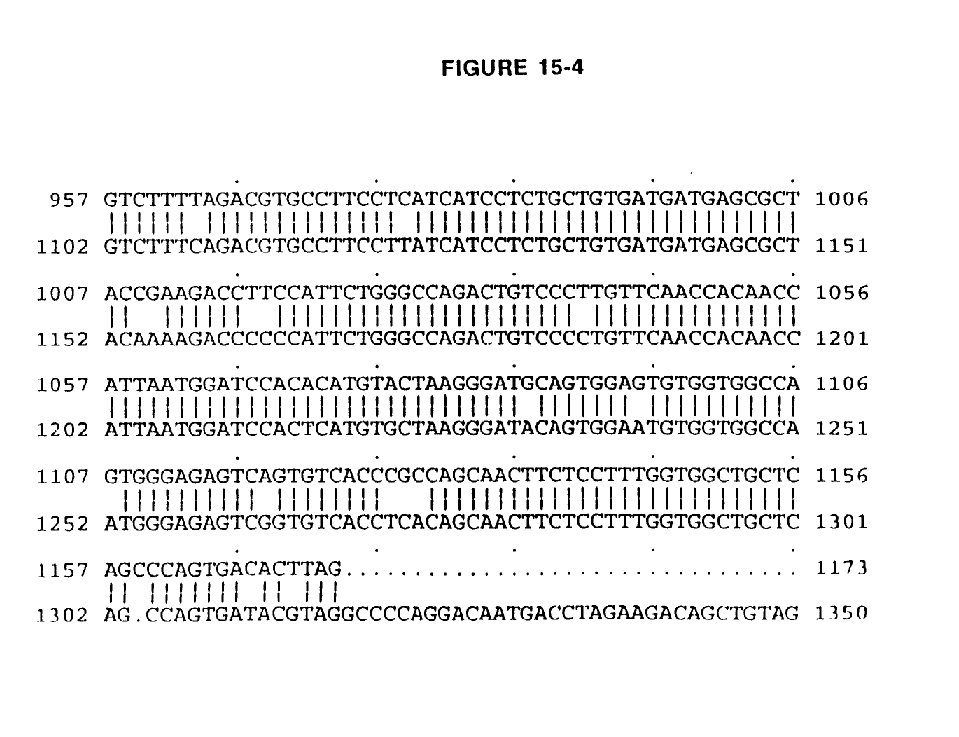

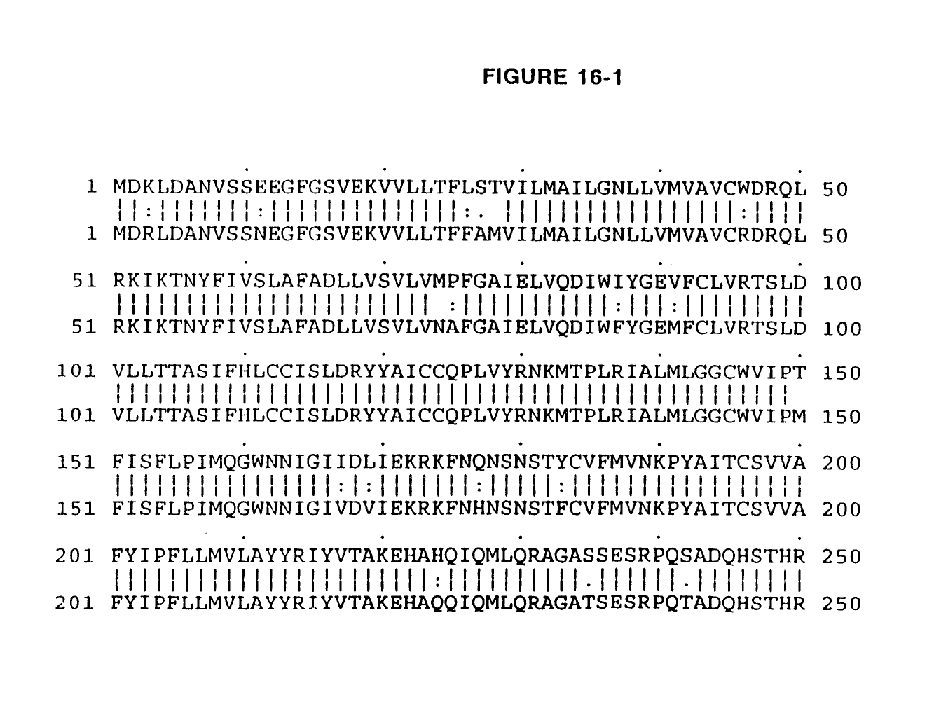

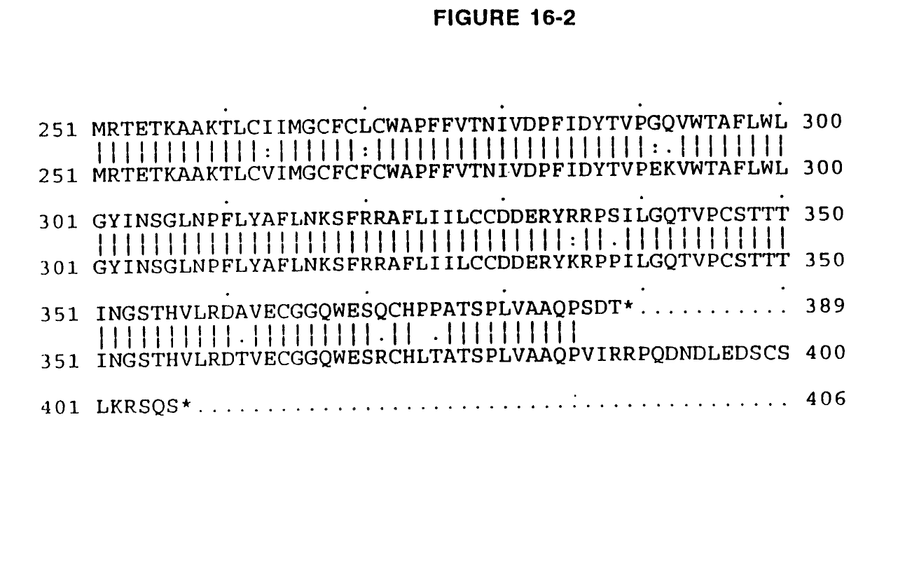

The full length human S10-95 nucleotide (FIG. 14A) and deduced amino acid sequences (FIG. 14B) are shown. Compared to the rat S10-95 sequence, it shows 90.7% identity at the nucleotide level and 91.8% identity at the amino acid level (FIGS. 15 and 16 respectively). The human S10-95 nucleotide sequence contains one nucleotide insertion in position 1159. This insertion creates a frame shift and introduces a stop codon in the reading frame 16 nucleotides downstream. The protein motifs are highly conserved between the rat and human homologs except for a casein kinase II potential phosphorylation site in position 288 which is lost in both human receptors. The human homologs both carry a potential cAMP/cGMP phosphorylation site in position 338 in their carboxy terminal tail which is absent in the rat homologs. A comparison of the amino acid sequence between the human and the rat S10-95 clones beginning from the initiating methionine and ending with the stop codon of the human S10-95 clone, reveals 31 amino acid changes of which 11 are non conservative, including 2 in TM1, 1 in TM2 and 1 in TM4. Due to the nucleotide insertion and the corresponding frame shift described above, the carboxy terminal tail of the human S10-95 receptor is 16 amino acid shorter than its rat homolog.

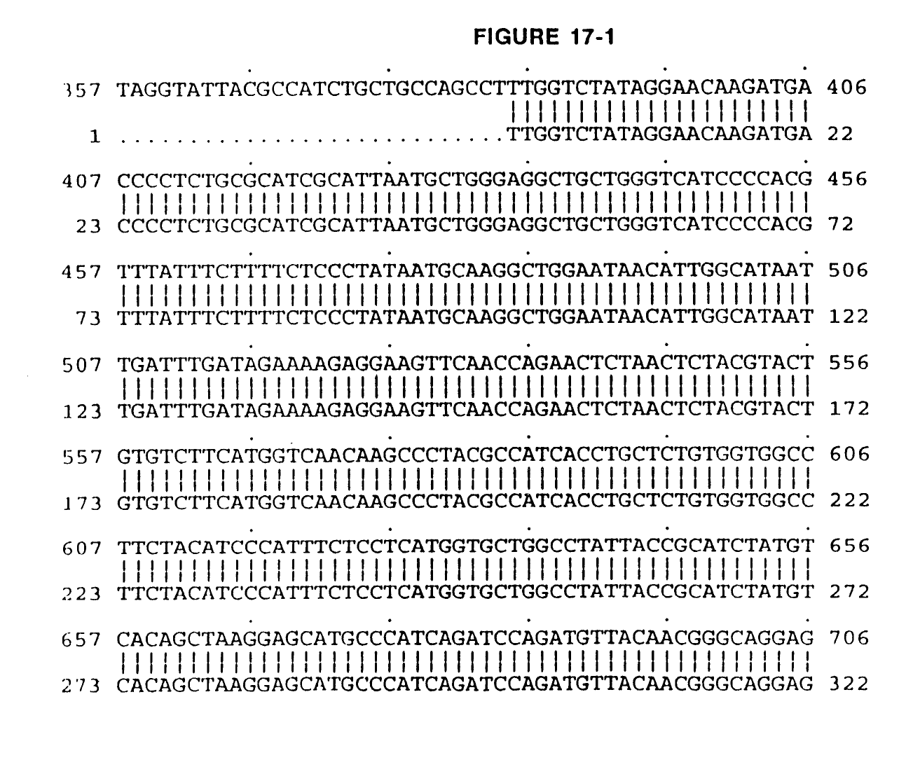

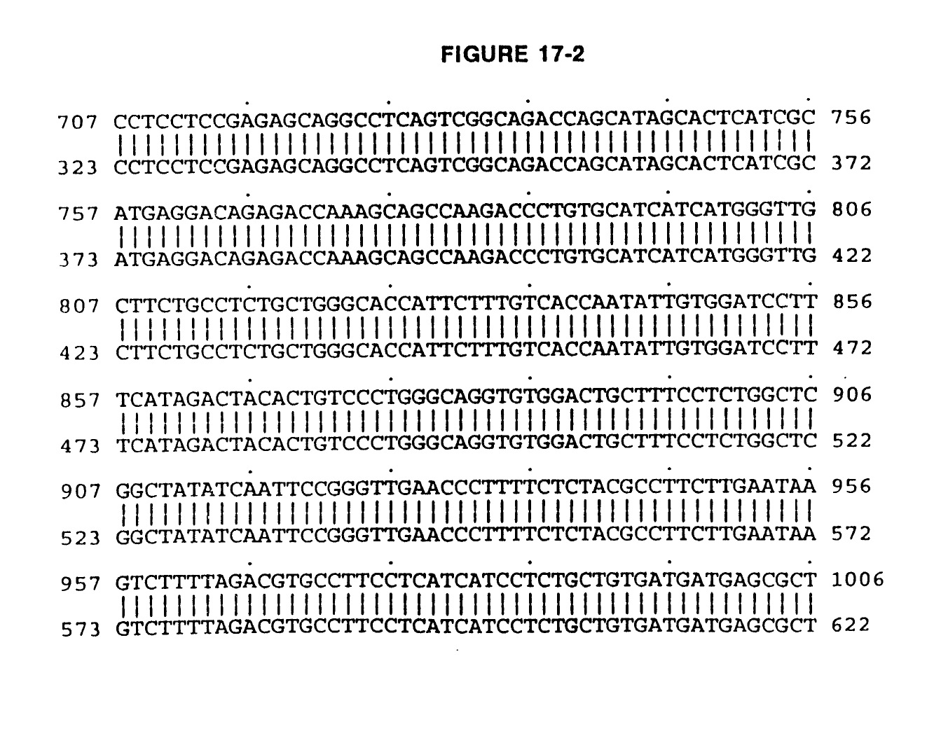

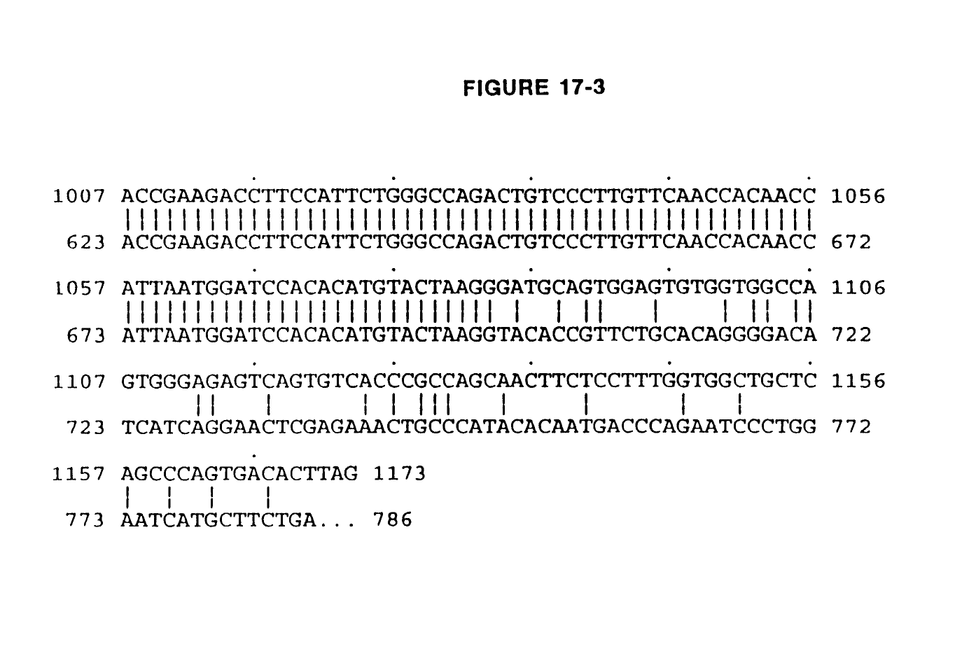

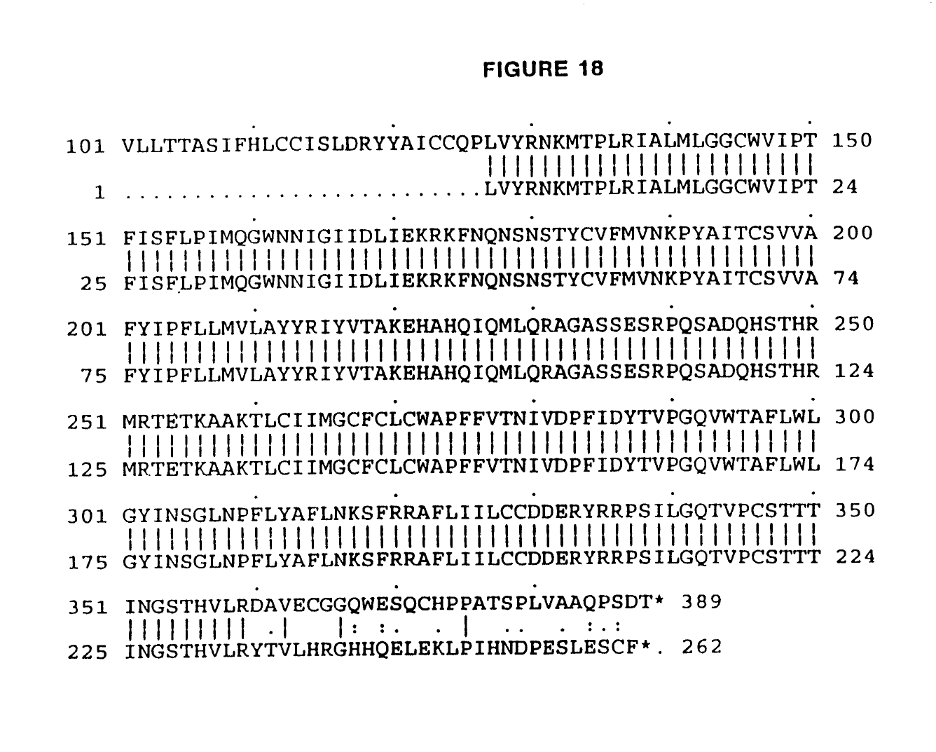

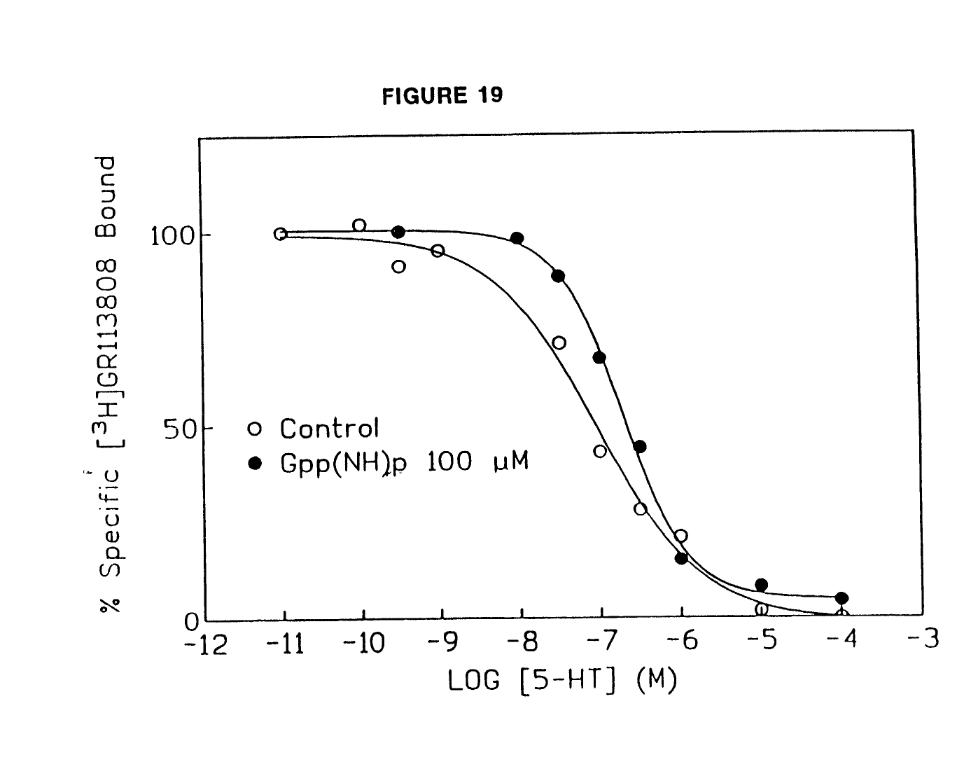

Identical to the rat homologs, both human clones are identical in the loops and transmembrane regions, differing only in the second half of their carboxy terminal tail (FIG. 17, nucleic acid sequence; FIG. 18, aa sequence).

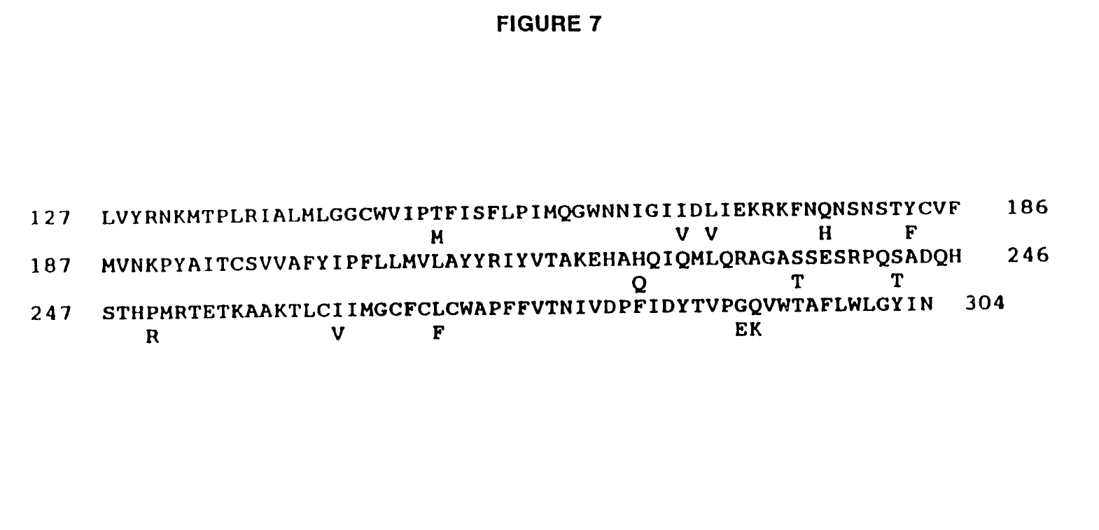

The human PCR cDNA fragments (TM-3 to TM-7) are 100% identical between heart, brain and retina. The nucleotide and deduced amino acid sequences are shown in FIG. 5. The human sequence shows 90.7% homology with the rat nucleotide sequence (FIG. 6) and 92.3% homology (FIG. 7) with the rat amino acid sequence.

The genes encoding the rat S10-87 and S10-95 receptors were transiently expressed in Cos-7 cells for pharmacological evaluation. Initial experiments using 5 nM [³H]5-HT indicated that both S10-87 and S10-95 were serotonergic sites as demonstrated by the degree of specific binding and density of sites expressed in the transfected cells when compared against the mock transfected controls. Saturation analysis of S10-87 (CG-7) was performed using 10 concentrations of [³H]5-HT (1-100 nM) and yielded a Bmax of 1,938═399 fmol/mg of protein and a K_{d} for [³H]5-HT of 7.87±0.06 nM. The degree of specific binding at concentrations of [³H]5-HT close to the K_{d} ranged from 70-84% throughout the experimental series (including saturation and competition studies). Although the use of [³H]5-HT as a radioligand to label 5-HT₄ receptors in brain tissue is not efficient due to the nonselectivity of the ligand, it became clear in the present studies using a homogeneous receptor population that [³H]5-HT would label this particular receptor. In fact, [³H]5-HT appears to be labelling the high affinity state of the 5-HT₄ receptor which is not unusual for the conditions upon which this receptor has been studied. Similar results using an agonist radioligand have been previously reported for the cloned 5-HT₂ receptor (Branchek et al., 1990).

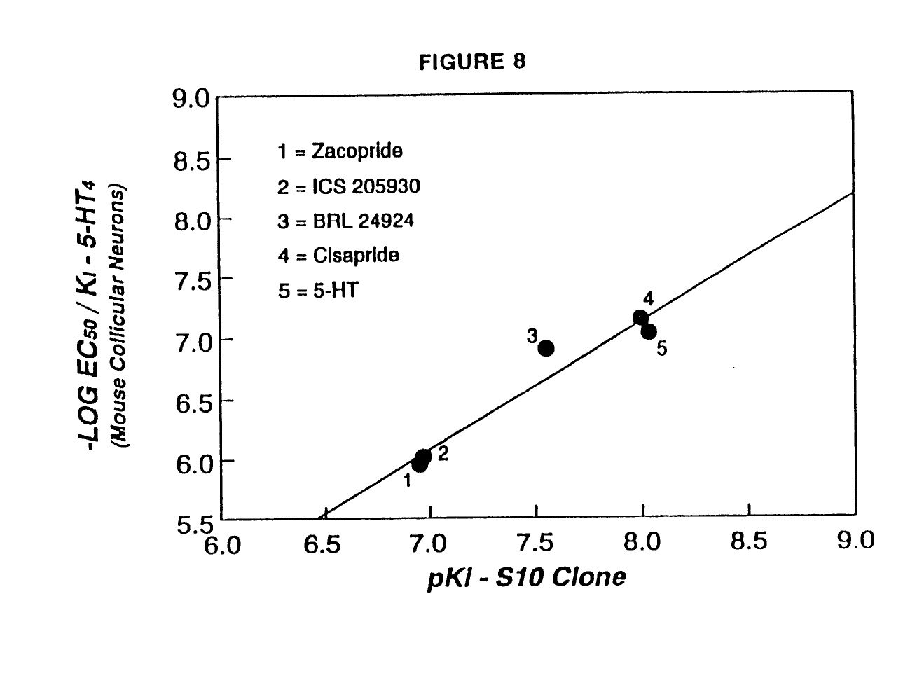

A pharmacological binding profile of S10-87 and S10-95 (CG-7 and CG-8) was performed and demonstrated that this novel receptor was similar to the 5-HT₄ receptor as defined by functional assays in the literature (Bockaert et al., 1992). This is clearly shown in table 3 where the binding affinities of various serotonergic agents are displayed for S10. Notably, 5-HT and the tryptamine derivative 5-methoxytryptamine possessed high affinity. Furthermore, as previously reported for the 5-HT₄ receptor, benzamide derivatives including cisapride, BRL 24924 and zacopride bound with fairly high affinity to receptors expressed from the S10 gene. ICS 205930, a tropanyl-indole derivative, which has been reported to be an antagonist at both 5-HT₃ and 5-HT₄ receptors (Bockaert et al., 1992), also bound to these receptor sites. Compounds such as 8-hydroxy-2-(di-n-propylamino)tetralin, ketanserin, sumatriptan and 5-carboxyamidotryptamine were of low affinity having K_{f} values estimated to be greater than 1 μM. This data would rule out S10 from belonging to other serotonergic receptor subfamilies such as 5-HT₁ and 5-HT₂. Taken together, the complete pharmacological profile also differentiates S10 from the related subtype 5-HT_{4B} (U.S. Ser. No., 971,960, filed, Nov. 3, 1992, copending). Although some of the drugs tested also have good affinity for 5-HT₃ receptors, S10 is clearly a 5-HT₄ receptor based upon the binding data and the functional data demonstrating a positive-coupling to adenylate cyclase. Finally, a correlation plot for the binding affinities of 5-HT, cisapride, BRL 24924, zacopride, and ICS 205930 against their functional responses in adenylate cyclase assays from cultured mouse collicular neurons (Dumuis et al., 1989) yielded a correlation coefficient of 0.96 (FIG. 8). Thus, the rank order of potency for these key compounds also provides conclusive evidence that S10 encodes a 5-HT₄ receptor.

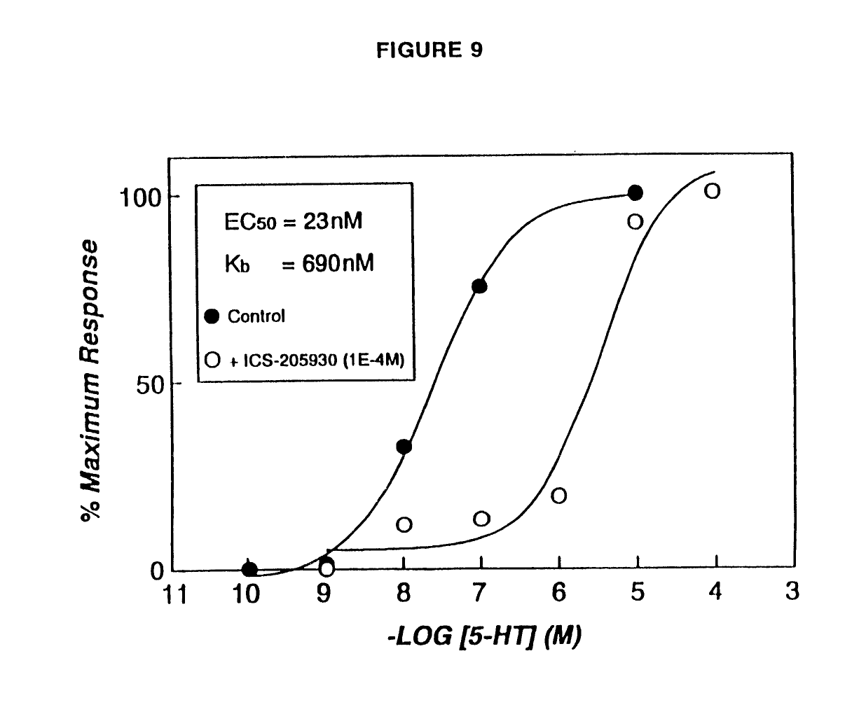

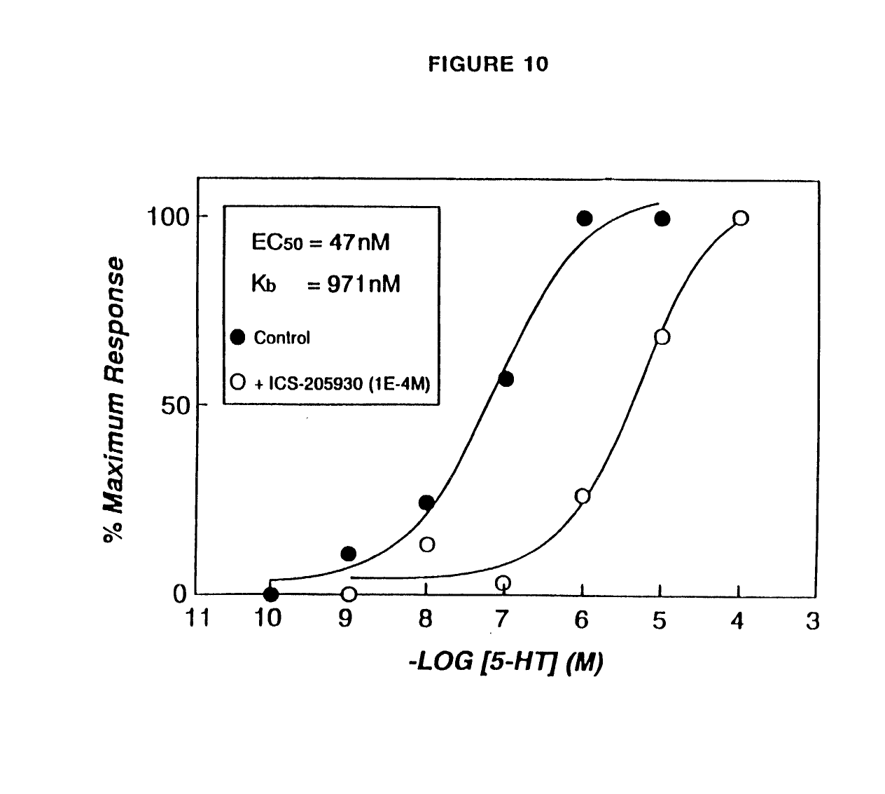

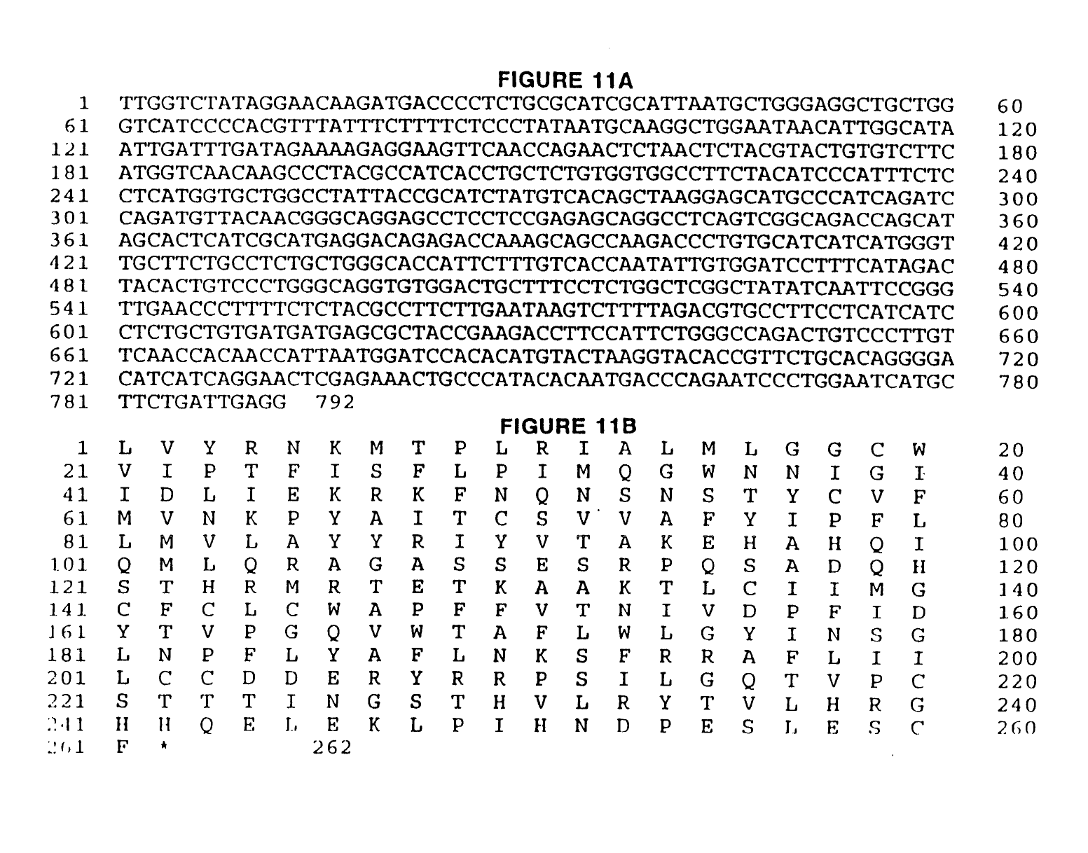

To examine the ability of S10 clone to couple to adenylate cyclase, Cos-7 cells transiently expressing S10 were tested for the ability to exhibit an increase in basal cAMP release or a decrease in FSK-stimulated cAMP response. 5-HT (1 μM) had no effect on either basal or FSK-stimulated adenylate cyclase activity in untransfected or mock-transfected Cos-7 cells (data not shown), indicating that endogenous cyclase-coupled serotonin receptors are not present in untransfected cells. Preliminary studies were carried out by adding only one dose of various drugs in triplicate. Addition of 5-HT (1 μM) to this system caused stimulation of basal cAMP release (CG-7=0.020±0.002; CG-8=0.023±0.004 pmol/ml/10 min) by about 30 fold for each clone; no inhibition of either the basal or FSK-stimulated cAMP release was observed. On the contrary, addition of 10 μM FSK together with 1 μM 5-HT stimulated cAMP accumulation about 10-fold more than either agent alone (data not shown). For various compounds, full dose-response curves were determined for both clones and the data are summarized in table 4. 5-HT caused a concentration-dependent stimulation of basal adenylate cyclase activity with mean EC₅₀s of 26±3 and 51±7 nM and Eₘₐₓs of 2,107 and 2,598% basal cAMP release for CG-7 and CG-8 respectively (FIGS. 9 and 10). Among the tryptamine derivatives tested, 5-MeOT was approximately equipotent with 5-HT in both clones, whereas α-Me-5-HT and 5-CT were about 10 and 200 times respectively less potent than 5-HT at CG-7. On the other hand, the latter two compounds displayed approximately 20 and 30 fold lower affinity than 5-HT respectively for CG-8. The 2-methoxy-4-amino-5-chloro-substituted benzamides (cisapride, BRL-249245 and zacopride) were less potent agonists than 5-HT in stimulating basal cAMP release and displayed different rank order of potency for CG-7 and CG-8. As indicated in table 4 using CG-7, cisapride, BRL-24924 and zacopride exhibited approximately 10, 30 and 100 fold lower potency than 5-HT respectively, whereas at CG-8 these compounds had almost equal affinity. Thus, although not different in binding properties, these subtle differences in affinity in functional assays of the two “variants” (CG-7 and CG-8) indicate the potential to develop separate therapeutic entities for each separate target. All the agonists tested acted as full agonists with the exception of cisapride, BRL-24924 and zacopride, which also displayed antagonist activity and were therefore partial agonists at both clones, with intrinsic activities ranging between 0.85 and 1.4 (Table 4). ICS-205-930 (100 μM) had similar effect at the two clones and was found to be a silent antagonist causing parallel dextral shifts in the concentration effect curve of 5-HT without altering the maximum response significantly. The estimated K₈ value for ICS-205-930 was not significantly different between the two clones (CG-7=962±244 nM: CG-8=607±30 nM). Responses to 5-HT were not affected by spiperone or methiothepin (10 μM) in either clone.

Saturation analysis of rat 5-HT_{4A} S10-87 (CG-7) and S10-95 (CG-8) clones and human 5-H^{T}_{4A} clone CG-17 were performed using 10-12 concentrations of [³H]GR113808 (0.005-2.5 nM) and revieled a single saturable site of high affinity for both clones (CG-7: K_{d}=0.74 nM, Bₘₐₓ=5.7 pmol/mg membrane protein: CG-8: K_{d}=1.0 nM, Bₘₐₓ=3.7 pmol/mg membrane protein; CG-17: K_{d}=0.20 nM, Bₘₐₓ=1.8 pmol/mg membrane protein). These preliminary data indicate that although the rat clones (CG-7 and CG-8) have similar affinity for the antagonist [³H]GR113808, the human clone (CG-17) displays approximately 5-fold higher affinity than the rat clones for this ligand. For all three clones nonspecific binding increased linearly with increasing ligand concentration. The degree of specific binding at concentrations of [₃H]GR113808 (0.4-0.5 nM) ranged from 80-90%).

The pharmacological binding profile of S10-87 and S10-95 (CG-7, CG-8, respectively) was investigated in displacement studies using [³H]GR113808 and/or [³H]5-HT. In order to compare CG-17 pharmacology with that previously obtained for the rat clones, CG-7 and CG-8, displacement studies on the human CG-17 clone were performed using [³H]5-HT as the radioligand.

A range of 5-HT₄ receptor agonists and antagonists completely inhibited the specific binding of [³H]GR113808 on both the rat CG-7 and CG-8 clones. Affinity values and Hill slopes derived from the curves using computer analysis are presented in Table 5. As previously observed using [³H]5-HT as the radioligand, the rat CG-7 and CG-8 receptors had very similar pharmacology. Of the agonists tested, only those active in 5-HT₄ containing preparations (5-HT and 5-MeOT) potently inhibited [³H]GR13808. By contrast, agonists for other 5-HT receptors, for example 5-HT_{1A} receptor agonist, 8-OH-DPAT, the 5-HT_{1D} receptor agonist, sumatriptan, the 5-HT₂ receptor antagonist, ketanserin, had no effect on [³H]GR113808 binding at concentrations up to 1 μM. The substituted benzamides, cisapride, BRL-24924 and zacopride, partial agonists at 5-HT₄ receptor all potently inhibited [³H]GR113808 binding. Specific [³H]GR113808 binding was also inhibited by the 5-HT₄ receptor antagonist ICS-205930.

For both the rat CG-7 and CG-8 clones, Hill slopes for the inhibition of [³H]GR113808 binding by 5-HT₄ receptor agonists but not the antagonist, ICS-205930, were shallow in the absence of Gpp(NH) with the exception of 5-CT, and α-Me-5-HT. For agonists that had shallow displacement curves, the binding was resolved into high and low affinity components and these are summarized in Table 5. The Kᵢ values obtained for the high affinity state of the receptor using [³H]GR113808 as the radioligand were compatible with the Kᵢ values obtained previously using [³H]5-HT as the radioligand which labels the high affinity state of the receptor (Table 5 and 6). Some differences were observed for the Kᵢ values of high affinity state of CG-7 compared to CG-8 (Table 3) and their nH values. For example, although there were no differences in the Kᵢ values of CG-7 and CG-8, the displacement curve obtained for 5-MeOT using CG-8 clone could not be resolved into two sites. Also the Kᵢ value obtained for the high affinity state of CG-8 using cisapride was approximately 3-fold lower than that obtained for CG-7 previously using [³H]5-HT to directly label the high affinity state of the receptor. We are currently investigating these differences using [³H]5-HT to directly label the high affinity state.

In the presence of 100 μM Gpp(NH)p, competition binding curves for the agonists displaying shallow curves in the absence of Gpp(NH)p were shifted to the right and this is exemplified for 5-HT in FIG. The Hill slopes were increased.

Preliminary results obtained with the human clone (CG-17) using [³H]5-HT as the radioligand in displacement studies are summarized in Table 3. Similar to the rat CG-7 and CG-8 clones, 8-OH-DPAT, sumatriptan and ketanserin were inactive at the CG-17 clone for up to concentration of 1 μM. The differences observed between the human and the rat CG-8 clones were as follows. The biggest difference was observed with α-Me-5-HT which had approximately 100 fold higher affinity for the human CG-17 clone. Zacopride, 5-MeOT and cisapride had about 7-fold, 5-fold and 4-fold higher affinity, respectively for the human clone CG-17.

Discussion

We have identified two cDNA clones encoding the pharmacologically-defined 5-HT₄ receptor. This receptor is expressed at low levels in rat brain if we consider its frequency in the cDNA library (≦1:10⁶). Surprisingly, two receptors differing in their carboxy-terminus regions have been isolated. Since the 3′ untranslated nucleotide sequences are also different, these two receptors could be encoded by two different genes or could arise by alternative splicing of pre-mRNA. These two receptors (S10-87 and S10-95) are differentially expressed in rat brain and the physiological relevance of the S10-87 receptor being expressed only in striatum remains to be determined.

The pharmacology binding profile and the functional coupling obtained from cells expressing S10 clones indicate that these genes both encode a pharmacologically-defined 5-HT₄ receptor. The cloned rat CG-7 and CG-8 genes transiently expressed in Cos-7 cells coupled to stimulation of adenylate cyclase. The magnitude of this response (−20-25 fold) was large. With the exception of 5-MeOT, agonist potencies determined from functional assays were less than expected from Kᵢ values obtained from binding assays using [³H]5-HT. Could this result be due to the possibility that the dose of [³H]5-HT used in binding assays measures only the high affinity site of agonists? This is not likely, as it would not account for the data obtained with ICS-205-930 which is a silent antagonist in the present system displaying approximately 6 (CG-8) and 10 (CG-7) fold lower affinity in the functional assay as compared to the binding experiments. It is more likely that differences in experimental conditions used in binding assays compared with those used in the functional assays (such as membrane vs. cells, buffers and extent of equilibrium achieved) accounts for these differences.

5-HT responses were resistant to blockade by methiothepin and spiperone (10 μM). As the concentration of these agents exceed their equilibrium dissociation constants for their respective receptor sites by 10-100 fold, it seems that 5-HT₁-like (5-HT_{1A}, 5HT_{1B}, 5BH_{1C}, 5HT_{1D}, 5HT_{1E}, 5-HT_{1F}), 5-HT₂ and 5-HT_{4B} receptors can be ruled out. In addition, the weak agonistic activity of 5-CT relative to 5-HT further supports the notion that 5-HT₁-like receptors are probably not involved (Bradley, 1986). The results obtained with the indole agonists reflect those reported at the 5-HT₄ receptor in both the CNS and the periphery (Dumuis et al., 1988; Craig and Clarke, 1990; Eglen et al., 1990; Baxter, Craig and Clarke, 1991). The substituted benzamides, cisapride, BRL-24924 and zacopride acted as partial agonists. Although the benzamides also possess affinity for 5-HT₃ receptors, they lack intrinsic efficacy (Schuurkes et al., 1985; Sanger and King, 1988). Furthermore, the affinity of ICS-205-930 for antagonism of 5-HT response at S10 is 1-3 orders of magnitude lower than that at 5-HT₃ receptors (Richardson et al., 1985) and therefore indicates a binding site different from 5-HT₃ receptor.

As was the case with the rat 5-HT₄ receptor, there are two forms of the human homolog, most likely splice variants differing in the carboxyterminal end of the receptor. Non conservative amino acid substitutions, especially in transmembrane domains 1, 2 and 4 could provide the basis for the pharmacological differences observed between the rat and the human 5-HT₄ receptors (see below). A difference in the functional response is observed between the two rat clones: although the CG-7 construct (S10-87) gives higher levels of receptor expression in COS-7 cells (Bmax of 5.7 pmol/mg of protein versus 3.7 pmol/mg for S10-95), in the functional assay, the CG-8 construct (S10-95) reproducibly shows a higher level of cAMP stimulation (2598±154% of basal cAMP release versus 2107±52% for CG-7). This finding could be attributed to the different amino acid sequence in the carboxy terminal tail of the rat receptors, specially since the rat S10-95 isoform carries an additional potential phosphorylation site at position 400, absent in S10-87 (CG-7).

Since the human S10-95 homolog lacks the last 16 carboxy terminal amino acids which carry the phosphorylation site mentioned above in the rat homolog, it will be interesting to check for differences in the level of cAMP stimulation upon activation of the rat and human S10-95 homologs. In the same way, after we get the full length human S10-87, both human isoforms will be compared in binding and functional assays.

All the unique pharmacological characteristics described above define the S10 genes as adenylate cyclase stimulatory “5-HT₄” receptors reported in the literature that are expressed in the ileum (Craig and Clarke, 1990), hippocampus (Shenker et al., 1987), esophagus (Baxter et al., 1991), embryo colliculi neurons (Dumius et al., 1988), atrium (Kaumann et al., 1990), adrenal (Lefebvre et al., 1992) and bladder (Corsi et al., 1991), and distinguish these clones from all other cloned subtypes of 5-HT receptor. Although the binding profile of CG-7 and CG-8 were identical (Table 3), some differences in agonist potency (benzamides in particular) were observed between them in the functional assays. This is not surprising since the amino acid sequences of these two clones are identical, apart from the cytoplasmic carboxy tail, a region that is important for G protein-coupling, where the CG-8 receptor carries an extra phosphorylation site. Cisapride, BRL-24924 and zacopride had similar affinities at CG-8 whereas BRL-24924 and zacopride displayed approximately 4 and 15 fold lower affinity than cisapride at CG-7 clone for stimulation of adenylate cyclase. It is noteworthy that tissue differences in the potency of benzamides have been reported (Baxter et al., 1991) and whether this reflects a heterogeneity of 5-HT₄ receptors remains to be investigated.

The data obtained with the rat CG-7 and CG-8 clones and the human CG-17 clone using [³H]GR113808 are very similar to those reported by Grossmann et al. (1993) and Waeber et al. (1993) with this ligand using guinea pig and human brain tissues. Specific [³H]GR113808 binding readily saturated for all three clones (CG-7, CG-8 and CG-17). Scatchard analysis of specific binding in all three clones revealed the involvement of a single site. Curve fitting analysis showed an equilibrium dissociation of approximately 1 nM for both rat clones (CG-7 and CG-8) whereas this value was about 5-fold lower for the human CG-17 clone (K_{d}=0.2 nM). The K_{d} value obtained for the human CG-17 clone is in excellent agreement with that reported by Waeber et al. (1993) using human brain (0.23-0.37 nM) and is very similar to that of the guinea pig brain tissue (0.13-0.2 nM: Waeber et al., 1993; Grossmann et al., 1993). The K_{d} value of [³H]GR113808 for rat brain tissue has not been reported, however, it is interesting that the affinity obtained for GR113808 from functional receptor assays in the rat oesophagus (Grossmann et al., 1993) is about 0.3 nM which indicates that this antagonist has similar affinities for the human and the rat tissue used. The discrepancies between our data and those reported may be due to methodology, or different subtype (brain vs. oesophagus), however, this remains to be investigated.

The rank order of potency of compounds competing for specific [³]GR113808 are very similar for both CG-7 and CG-8 and is cisapride>5-HT>BRL-24924>5-MeOT=ICS205930>zacopride>α-Me-5-HT>5-CT. This order of potency is different from that observed with guinea pig caudate (Grossmann et al., 1993; cisapride>5-HT>ICS205930>BRL-24924>zacopride>5-MeOT>α-Me-5-HT>5-CT) and human caudate (Waeber et al., 1993; cisapride>ICS205930>BRL-24924>5-HT>5-MeOT. Whether these differences are due to species or different population of high affinity state of the receptor in the various preparation, remains to be investigated. Interestingly, the displacement curves for most of the agonists competing for specific [³H]GR113808 were shallow and could be resolved to high and low affinity states. Gpp(NH)p shifted these curves to the right and in the case of 5-HT the Hill coefficient was increased to unity; however for some agonists the shift was not complete. Grossmann et al.(1993) using guinea pig caudate also observed shallow competition curves for some agonist that could be partially shifted by the addition of GTP (Grossmann et al., 1993). However, Waeber did not observe shallow binding curves with the human caudate tissue. These observations indicate that the G protein content of these prepartations may be different which may reflect differences in the coupling of the receptor with a G protein.

Using [³H]5-HT as the radioligand, the affinity values for the human CG-17 clone are in general comparable with that obtained by us previously tor the rat CG-7 and CG-8 clones with few exceptions. The most striking differences appears to be for α-Me-5-HT which displays approximately 100 fold higher affinity for the human CG-17. However, it has to pointed out that the data are compared with 2 different radioligands and this difference has to be further investigated using the same radioligand for all both the rat and human clones in parallel. 5-MeOT was approximately 3 fold less potent whereas zacopride was about 7 fold more potent at the human CG-17 as compared to the rat clones.

The cloning and expression of genes encoding 5-HT₄ receptors allows, for the first time, the ability to develop subtype selective drugs using radioligand binding assays. It will further provide definitive answers to whether there are significant species differences in the pharmacology of 5-HT₄ receptors. In addition, the intrinsic activity of drugs can be determined from measures of adenylate cyclase activation in these transfected cells. So far, native tissue preparations have shown great disparity in agonist activity. 5-HT₄ receptors have been implicated in a wide variety of functions. Existing drugs such as metaclopramide and cisapride appear to exert a large part of their action through 5-HT₄ receptors (Taniyama et al., 1991; Meulemans and Schurkes, 1992; Flynn et al., 1992). Experience with these agents indicates a clear therapeutic role for 5-HT₄ receptors in the gastrointestinal system for conditions including irritable bowel disease, postoperative ileus, diabetic gastroparesis, emesis, achalasia, hiatal hernia, and esophageal spasm. In addition, 5-HT₄ receptors have been described functionally in the heart (Kaumann, 1992), adrenal (Lefebvre et al., 1992), and bladder (Corsi et al., 1991) indicating possible roles in cardiac rate and force of contraction, endocrine control of cortisol secretion, and urinary incontinence or spasticity. 5-HT₄ receptors have also been described in the brain, particularly in areas such as the hippocampus, in which we have localized the gene encoding 5-HT₄ receptors (S10-87), indicating a potential role in cognitive enhancement (Bockaert et al., 1992). As more specific pharmacological tools are developed, additional therapeutic indications will certainly be uncovered.

TABLE I
% TM homology of the S10 receptor with other 7 TM receptors

| SEROTONIN | | ADRENERGIC | DOPAMINE | PEPTIDE | |
|---|---|---|---|---|---|
| 5-HT_{1A} | Hu 44 | Alpha-1A Hu 45 | D₁ Hu 43 | Subst K | 25 |
| | | | | Hu | |
| 5-HT_{1Dα} | Hu 40 | Alpha-1B Hu 43 | D₂ Hu 42 | TSH | 27 |
| 5-HT_{1Dβ} | Hu 41 | Alpha-1C Hu 40 | D₃ Rt 46 | | |
| 5-HT_{1E} | Hu 41 | Alpha-2A Hu 42 | D₄ Hu 45 | | |
| 5-HT_{1F} | Hu 41 | Alpha-2B Hu 40 | D₅ Hu 45 | | |
| 5-HT₂ | Hu 42 | Alpha-2C Hu 40 | | | |
| 5-HT_{1C} | Hu 44 | | | | |
| | | Beta-1 Hu 46 | HISTAMINE | MUSCARINIC | |
| 5-HT Dro | S 43 | Beta-2 Hu 44 | | | |
| 5-HT Dro | I_{A} 40 | Beta-3 Hu 42 | H₁ Bov 36 | m1 | 35 |
| 5-HT Dro | I_{B} 41 | | H₂ Hu 46 | | |
| 5-HT_{4B} | Hu 44 | | | | |
| | | | ADENOSINE | | |
| | | | A1 Rat 32 | | |
| | | | A2 Hu 31 | | |

TABLE 2
Table 2: PCR localization of the S10 mRNA in 7 different part of the rat brain
The TM3-5 primers do not differentiate between clones S10-87 and S10-95.
The S10-87 primers were designed from the nucleotide sequences coding
for the TM 6 domain common to both receptors and for the carboxy terminus
end specific to S10-87. In the same way, the S10-95 primers are specific
for S10-95.

| | Corte | Cerebell | Brain | Hippocamp | Olfactory | Striatu | Thalam |
|---|---|---|---|---|---|---|---|
| Primers | x | um | Stem | us | Bulb | m | us |
| TH3-5 | + | − | + | + | + | + | + |
| S10-97 | ND | − | − | − | − | + | − |
| S10-95 | ND | − | + | + | + | + | + |

TABLE 3
Binding affinities of key ligands for the identification of S10
(CG-7 and CG-8) as a 5-HT₄ receptor. Affinity constants (Ki; nM) of
drugs competing for S10 labeled with 10 nK [3H⁴]5-HT were determined
to pharmacologically define the encoded receptor as 5-HT₄. Ki (nM)
values were calculated using the Cheng-Prusoff equation or estimated
to be >1000 nM based upon one point displacements using a drug
concentration of 1 uM. Affinity constants are expressed as the
mean ± SEM (n ≦ 2). Ki values estimated to be >1000 were
determined according to one point displacements studies at a
concentration of 1 uM. (n = 2 except BRL 24924 tested at
CG-8: n = 1)
CHARACTERIZATION OF CLONE S-10

| | |
|---|---|
| Saturation Analysis: | Kd = 7.87 ± 0.06 nM |
| | Bmax = 1,938 fmol/mg prot |
| Pharmacological profile: | |

| DRUG | CG-7 | CG-8 |
|---|---|---|
| 5-HT | 8.6 ± 0.6 | 6.4 ± 0.5 |
| Cisapride | 10.9 ± 0.3 | ND |
| 5-MeOT | 27.5 ± 5 | ND |
| BRL 24924 | 27.7 ± 5 | 21.1* |
| ICS 205930 | 115 ± 12 | 138 ± 26 |
| Zacopride | 130 ± 10 | 136 ± 5 |
| BOHDPAT | >1000 | ND |
| Ketanserin | >1000 | ND |
| Sumatriptan | >1000 | ND |
| 5-CT | >1000 | ND |

*n = 1
ND = not determined

TABLE 4
Pharmacological profile for the cAMP response using the human 5-Ht_{4A} (CG-7 and CG-8)
receptor transiently expressed in Cos-7 cells, comparison with the binding data
obtained with CG-7 clone using [³H]5-HT. cAMP measurements on intact cells were
as described under Methods and Materials EC₅₀ values (concentration producing the
half-maximal effect) were derived from the analysis of full dose-response curves.
Maximum response produced by each drug was normalized to the 5-HT maximum
response which is indicated as having an intrinsic activity of 1.0. Data are means ±
S.E.M. of three separate experiments. The apparent dissociation constant of antagonist
(Kₛ) (ICS 205930) was calculated according to the formula: Kₛ = [B]/(A′/A)-1],
where [B] is the concentration of antagonist, A′ and A the EC₅₀ values
of agonist measured respectively in the presence and in the absence of
antagonist (Furchgott, 1972).

| | EC₅₀ or Kₛ (nM) | | I. A. * | | K₁ (nM) | K₁ (nM) |
|---|---|---|---|---|---|---|
| DRUG | CG-7 | CG-8 | CG-7 | CG-8 | CG-7 | CG-8 |
| 5-MeOT | 21 ± 6 | 31 ± 13 | 1.0 | 1.0 | 27 ± 5 | ND |
| 5-HT | 26 ± 3 | 51 ± 7 | 1.0 | 1.0 | 8.6 ± 0.6 | 6.4 ± 0.5 |
| Cisapride | 191 ± 26 | 413 ± 199 | 1.2 | 1.4 | 11 ± 0.3 | ND |
| α-Me-5-HT | 250 ± 91 | 1,038 ± 31 | 0.90 | 1.0 | ND | ND |
| BRL-24924 | 736 ± 129 | 250 ± 25 | 1.1 | 0.9 | 28 ± 5 | 21 |
| Zacopride | 2,740 ± 274 | 239 ± 33 | 1.1 | 1.0 | 130 ± 10 | 136 ± 5 |
| 5-CT | 5,570 ± 808 | 1,411 ± 211 | 0.85 | 1.2 | >1,000 | ND |
| ICS-205930 | 962 ± 244 | 607 ± 30 | 0 | 0 | 115 ± 12 | 138 ± 26 |

ND, not determined.
Maximum response to 5-HT in Cos-7 cells transiently transfected with CG-7 and CG-8 genes was:
CG-7 = 2, 107 ± 52% of basal cAMP release
CG-8 = 2,598 ± 154% of basal cAMP release
Cisapride, BRL-24924 and zacopride had no antagonist activity and ICS-205930 had no intrinsic agonist activity.

TABLE 5
The affinities of various compounds that compete for 0.2-0.4 nM
[³H] GR113808 binding in membranes of COS-7 cells
transiently transfected with rat clones CG-7 and CG-8.

| COM- | | | | |
|---|---|---|---|---|
| POUNDS | CG-7 | | CG-8 | |
| 5-HT | 237 | K_{H} = 2.6, BH = 22% | 116 | KH = 2.5, BH = 24% |
| | | K_{L} = 357 | | KL = 197 |
| | | nH = 0.62 | | nH = 0.67 |
| 5-CT | >10,000 | | >10,000 | |
| 5-Me-OT | 438 | KH = 14, BH = 17% | 518 | KH = , BH = |
| | | KL = 658 | | KL = |
| | | nH = 0.57 | | nH = 0.66 |
| BRL- | 189 | KH = 37, BH = 34% | 188 | KH = 23, BH = 32% |
| 24924 | | KL = 371 | | KL = 373 |
| | | nH = 0.85 | | nH = 0.81 |
| ZACO- | 729 | KH = 424, BH = 68% | 820 | KH = , BH = |
| PRIDE | | KL = 2,757 | | KL = |
| | | nH = 0.80 | | nH = 0.85 |
| d-LSD | >10,000 | | >10,000 | |
| CISA- | ND | | 84 | KH = 2.7, BH = 11% |
| PRIDE | | | | KL = 105 |
| | | | | nH = 0.83 |

| | | |
|---|---|---|
| ICS- | ND | 529 |
| 205930 | | |
| α-Me- | 2,255 | 1,855 |
| 5-HT | | |

Affinity estimates are given as K₁ values (nM) using the computer program “Accucomp” (Lundon Software). Values are from a single experiment. K_{H} and K_{L} values indicate the affinity in nM for the high affinity and low affinity state of the receptor, B_{H} is the percentage of high affinity sites and nH is the Hill coefficient. ND, not determined.

TABLE 6
Binding affinities of key ligands for the
identification of the human CG-17 clone as a 5-HT_{4A} receptor.

| COMPOUND | Kᵢ (nM) |
|---|---|
| 5-HT | 4.2 |
| 5-MeOT | 71 |
| 5-CT | >10,000 |
| Cisapride | 12 |
| α-Me-5-HT | 1.6 |
| BRL-24924 | 21 |
| Zacopride | 17 |
| Sumatriptan | >1,000 |
| 8-OH-DPAT | >1,000 |
| Ketanserin | >1,000 |

Affinity constants (Ki, nM) of drugs competing for CG-17 cloned labeled with 10 nM [³H]5-HT were calculated using the Cheng-Prusoff equation or estimated to be greater than 1,000 nM based upon one point displacement using a drug concentration of 1 μM. Values are from a single experiment.

### REFERENCES

Adham, N., P. Romanienko, P. Hartig, R. L. Weinshank, and T. Branchek. The rat 5-hydroxytryptamine_{1B} receptor is the species homologue of the human 5-hydroxytryptamine_{1Dβ} receptor. Mol. Pharmacol. 41:1-7 (1992).

Adham, N., H.-T. Kao, L. E. Schechter, J. Bard, M. Olsen, D. Urquhart, M. Durkin, P. R. Hartig, R. L. Weinshank, and T. A. Branchek. Cloning of a novel human serotonin receptor (5-HT1F): A fifth 5-HT₁ receptor subtype coupled to the inhibition of adenylate cyclase. Proc. Natl. Acad. Sci. USA, in press.

Bockaert, J., J. R. Fozard, A. Dumuis, and D. E. Clarke. The 5-HT4 receptor: a place in the sun. Trends Pharmacol. Sci. 13:141-145 (1992).

Bockaert, J., M. Sebben, and A. Dumius. Pharmacological characterization of 5-hydroxytryptamine₄ (5-HT₄) receptors positivly coupled to adenylate cyclase in adult guinea-pig hippocampal membranes: effect of substituted benzamide derivatives. Mol. Pharmacol. 37:408-411 (1990).

Bradford, M. M. A rapid and sensitive method for the quantification of microgram quantities of protein utilizing the principle of protein-dye binding. Anal. Biochem. 72:248-254 (1976).

Bradley, P. B., G. Engel, W. Fenuik, J. R. Fozard, P. P. Humphrey et al. Proposals for the nomenclature of functional receptors for 5-hydroxytryptamine. Neuropharmacology 25:563-576 (1986).

Branchek, T., N. Adham, M. Macchi, H.-T. Kao, and P. R. Hartig. [³H]-DOB (4-bromo-2,5-dimethoxyphenylisopropylamine) and [³H]ketanserin label two affinity states of the cloned human 5-hydroxytryptamine₂ receptor. Mol. Pharmacol. 38:604-609 (1990).

Capecchi, M. R., Science 244: 1288-1292 (1989).

Chirgwin, J. M., A. E. Przybyla, R. J. MacDonald, and W. J. Rutter. Isolation of biologically active ribonucleic acid from sources enriched in ribonuclease. Biochemistry 18:5294 (1979).

Cohen, J. S., Trends in Pharm. Sci. 10:435 (1989)

Cushing, D., and M. L. Cohen. Comparison of the serotonin receptors that mediate smooth muscle contraction in canine and porcine coronary artery. J. Pharmacol. Exp. Ther. 261:856-861 (1992).

Demchyshyn, L., R. K. Sunahara, K. Miller, M. Teitler, B. J. Hoffman, J. L. Kennedy, P. Seeman, H. H. M. Van Tol, and H. B. Niznik. A human serotonin 1D receptor variant (5-HT1Dβ) encoded by an intronless gene on chromosome 6. Proc. Natl. Acad. Sci. USA 89:5522-5526 (1992).

Dumuis, A., R. Bouhelal, M. Sebben, R. Cory, and J. Bockaert. A nonclassical 5-hydroxytryptamine receptor positively coupled with adenylate cyclase in the central nervous system. Mol. Pharmacol. 34:880-887 (1988).

Dumuis, A., M. Sebben, and J. Bockaert. (1989). The gastrointestinal prokinetic benzamide derivatives are agonists at the non-classical 5-HT receptor (5-HT₄) positively coupled to adenylate cyclase in neurons. Naunyn-Schmiedeberg's Arch. Pharmacol. 340:403-410 (1989).

Fargin, A., J. R. Raymond, M. J. Lohse, B. K. Kobilka, M. G. Caron, and R. J. Lefkowitz. The genomic clone G21, which resembles a β-adrenergic receptor sequence, encodes the human 5-HT_{1A} receptor. Nature (Lond.) 335:358-360 (1988).

Foquet, M., D. Hoyer, L. A. Pardo, A. Parekh, F. W. Kluxen, H. O. Kalkman, W. Stühmer, and H. Lübbert. Cloning and functional characterization of the rat stomach fundus serotonin receptor. EMBO J. 11(3):3481-3487 (1992).

Grossman, C. J., Kilpatrick, G. J. Aid Bunce, K. T. Development of a radioligand binding assay for 5HT4 receptors in guinea pig and rat brain. Br. J. Pharmacol. 109:618-624 (1993)

Gubler, U., and B. J. Hoffman. A simple and very efficient method for generating cDNA libraries. Gene 25:263 (1983).

Hamblin, M. W., and M. A. Metcalf. Primary structure and functional characterization of a human 5-HT_{1D}-type serotonin receptor. Mol. Pharmacol. 40:143-148 (1991).

Hartig, P. R., H.-T. Kao, M. Macchi, N. Adham, J. Zgombick, R. Weinshank, and T. Branchek. The molecular biology of serotonin receptors: an overview. Neuropsychopharmacol. 3:335-347 (1990).

Herrick-Davis, K. and M. Titeler. Detection and characterization of the serotonin 5-HT_{1D} receptor in rat and human brain. J. Neurochem. 50:1624-1631 (1988). Hogan, B., et al., Manipulating the Mouse Embryo, A Laboratory Manual, Cold Spring Laboratory (1986).

Jin, H., D. Oksenberg, A. Ashkenazi, S. J. Peroutka, A. M. V. Duncan, R. Rozmahel, Y. Yang, G. Mengod, J. M. Palacios, and B. F. O'Dowd. Characterization of the human 5-hydroxytryptamine_{1B} receptor. J. Biol. Chem. 267:5735-5738 (1992).

Kobilka, B. K., T. Frielle, S. Collins, T. Yang-Feng, T. S. Kobilka, U. Francke, R. J. Lefkowitz, and C. G. Caron. An intronless gene encoding a potential member of the family of receptors coupled to guanine nucleotide regulatory proteins. Nature 329:75-77 (1988).

Levy, F. O., T. Gudermann, M. Birnbaumer, A. J. Kaumann, and L. Birnbaumer. Molecular cloning of a human gene (S31) encoding a novel serotonin receptor mediating inhibition of adenylyl cyclase. FEBS. Lett. 296:201-206 (1992).

Low, M. J., R. M. Lechan and R. E. Hammer, Science 231: 1002-1004 (1986).

Maniatis, T., E.F. Fritsch, and J. Sambrook, Molecular Cloning Manual, Cold Spring Harbor Laboratory, Cold Spring Harbor N.Y. (1982).

Maricq, A. V., A. V. Peterson, A. J. Brake, R. M. Myers, and D. Julius. Primary structure and functional expression of the 5-HT3 receptor, a serotonin-gated ion channel. Science 254:432-436 (1991).

Maroteaux, L., F. Saudou, N. Amlaiky, U. Boschert, J. L. Plassat, R. Hen. Mouse 5-HT_{1B} serotonin receptor: cloning, functional expression, and localization in motor control centers. Proc. Natl. Acad. Sci. USA 89:3020-3024 (1992).

McAllister, G., A. Charlesworth, C. Snodin, M. S. Beer, A. J. Noble, D. N. Middlemiss, L. L. Iverson, and P. Whiting. Molecular cloning of a serotonin receptor from human brain (5-HT1) A fifth 5-HT1-like subtype. Proc. Natl. Acad. Sci. USA 89:5517-5521 (1992).

Miller, J., and R. N. Germain. Efficient cell surface expression of class II MHC molecules in the absence of associated invariant chain. J. Exp. Med. 164:1478-1489 (1986).

Mylecharane, E. and C. Phillips. Mechanisms of 5-hydroxytryptamine-induced vasodilation. In: The Peripheral Actions of 5-hydroxytryptamine, J. R. Fozard, ed. Oxford University Press, Oxford, pp. 147-181 (1989).

Oberdick, J., R. J. Smeyne, J. R. Mann, S. Jackson and J. I. Morgan, Science 248: 223-226 (1990).

O'Dowd, B. F., M. Hnatowich, M. G. Caron, R. J. Lefkowitz, and M. Bouvier. Palmitoylation of the Human β₂-Adrenergic Receptor. J. Biol. Chem. 264:7564-7569 (1989).

Pritchett, D. B., A. W. J. Bach, M. Wozny, O. Taleb, R. Dal Toso, J. C. Shih, and P. H. Seeburg. Structure and functional expression of a cloned rat serotonin 5-HT-2 receptor. EMBO J. 7:4135-4140 (1988).

Sambrook, J., E. F. Fritsch, and T. Maniatis. In: Molecular Cloning: A Laboratory Manual, 2nd Edition, Cold Spring Harbor Laboratory, Cold Spring Harbor, N.Y. (1989). Sarver, N., et al., Science 247: 1222 (1990).

Shenker, A., S. Maayani, H. Weinstein, and J. P. Green. Pharmacological characterization of two 5-hydroxytryptamine receptors coupled to adenylate cyclase in guinea pig hippocampal membranes. Mol. Pharmacol. 31:357-367 (1987).

Southern, E. Detection of specific sequences among DNA fragments seperated by gel electrophoresis. J. Mol. Biol. 98:503-517 (1975).

Trevethick, M. A., W. Feniuk, and P. P. A. Humphrey. 5-hydroxytryptamine induced relaxation of neonatal porcine vena cava in vitro. Life Sci. 35:477-486 (1984).

Waeber, C., Sebben, M., Grossman, C, Javoy-Agid, F., Bockaert, J. and Dumuis, A. [3H]-GR113808 labels 5-HT4 receptors in the human and guinea-pig brain. Neuroreport 4:1239-1242 (1993)

Warden, D., and H. V. Thorne. Infectivity of polyoma virus DNA for mouse embryo cells in presence of diethylaminoethyl-dextran. J. Gen. Virol. 3:371 (1968).

Weinshank, R. L., J. M. Zgombick, M. Macchi, N. Adham, H. Lichtblau, T. A. Branchek, and P. R. Hartig. Cloning, expression, and pharmacological characterization of a human α_{2B}-adrenergic receptor. Mol. Pharmacol. 38:681-688 (1990).

Weinshank, R. L., J. M. Zgombick, M. Macchi, T. A. Branchek, and P. R. Hartig. The human serotonin 1D receptor is encoded by a subfamily of two distinct genes: 5-HT_{10α} and 5-HT_{1Dβ}. Proc. Natl. Acad. Sci. USA 89:3630-3634 (1992a). Weintraub, H. M., Scientific American, January (1990) p.40.

Zhao D., J. Yang, K. E. Jones, C. Gerald, Y. Suzuki, P. G. Hogan, W. W. Chin, and A. H. Tashjian, Jr. Molecular cloning of a cDNA encoding the thyrotropin-releasing hormone receptor and regulation of its mRNA in rat GH cells. Endocrinology. 130:3529-3536 (1992).

Zemlan, F. P., and E. F. Schwab. Characterization of a novel serotonin receptor subtype (5-HT₁₅) in rat CNS: interaction with a GTP binding protein. J. Neurochem. 57:2092-2099 (1991).

Zgombick, J. M., R. L. Weinshank, M. Macchi, L. E. Schechter, T. A. Branchek, and P. R Hartig. Expression and pharmacological characterization of a canine 5-hydroxytryptamine_{1D} receptor subtype. Mol. Pharmacol. 40: 1036-1042 (1991).

Zgombick, J. M., L. E. Schechter, M. Macchi, P. R. Hartig, T. A. Branchek, and R. L. Weinshank. Human gene S31 encodes the pharmacologically defined serotonin 5-hydroytryptamine_{1E} receptor. Mol. Pharmacol. 42: 180-185 (1992).

Zimmer, A., and P. Gruss, Nature 338: 150-153 (1989).

What is claimed is:

1. An isolated nucleic acid molecule encoding a mammalian 5-HT₄ receptor.

2. A nucleic acid molecule of claim 1, wherein the nucleic acid molecule encodes a human 5-HT₄ receptor.

3. A nucleic acid molecule of claim 1, wherein the nucleic acid molecule is a DNA molecule.

4. A DNA molecule of claim 3, wherein the DNA molecule is a cDNA molecule.

5. A DNA molecule of claim 3 wherein the DNA molecule is genomic DNA.

6. A nucleic acid molecule of claim 2, wherein the nucleic acid molecule is a DNA molecule.

7. An isolated DNA molecule encoding a mammalian 5-HT₄ receptor having the sequence H₂N—Y—X—COOH wherein Y is the amino acid sequence beginning at amino acid 1 and ending at amino acid 359 of FIG. 1 (SEQ ID No. 2) and wherein X is an amino acid sequence encoding the carboxy terminal region of the receptor.

8. An isolated nucleic acid molecule of claim 7, wherein X is the amino acid sequence beginning at amino acid 360 and ending at amino acid 387 of FIG. 1 (SEQ ID NO. 2).

9. An isolated nucleic acid molecule of claim 7, wherein X is the amino acid sequence beginning at amino acid 360 and ending at amino acid 406 of FIG. 2 (SEQ ID NO. 4).

10. An isolated nucleic acid molecule of claim 7, wherein Y is encoded by the nucleotide sequence from nucleotide 101 to nucleotide 1177 of FIG. 1 (SEQ ID NO. 1).

11. An isolated nucleic acid molecule of claim 8, wherein X is encoded by the nucleotide sequence from nucleotide 1178 to nucleotide to 1261 of FIG. 1 (SEQ ID NO. 1).

12. An isolated nucleic acid molecule of claim 9, wherein X is encoded by the nucleotide sequence from nucleotide 1127 to nucleotide 1267 of FIG. 2 (SEQ ID NO. 3).

13. A vector comprising a cDNA molecule of claim 4.

14. A plasmid vector of claim 13.

15. A vector of claim 13 adapted for expression in a bacterial cell which comprises regulatory elements necessary for expression of the cDNA encoding a 5-HT₄ receptor in the bacterial cell operatively linked to the cDNA encoding the 5-HT₄ receptor as to permit expression thereof.

16. A vector of claim 13 adapted for expression in a yeast cell which comprises the regulatory elements necessary for the expression of the cDNA encoding a 5-HT₄ receptor in the yeast cell operatively linked to the cDNA encoding the 5-HT₄ receptor as to permit expression thereof.

17. A vector of claim 13 adapted for expression in an insect cell which comprises the regulatory elements necessary for expression of the cDNA encoding a 5-HT₄ receptor in the insect cell operatively linked to the cDNA encoding the 5-HT₄ receptor as to permit expression thereof.

18. A vector of claim 13 adapted for expression in a mammalian cell which comprises the regulatory elements necessary for expression of the cDNA encoding a 5-HT₄ receptor in the mammalian cell operatively linked to the cDNA encoding the 5-HT₄ receptor as to permit expression thereof.

19. A plasmid of claim 14 adapted for expression in a mammalian cell which comprises the regulatory elements necessary for expression of the DNA in the mammalian cell operatively linked to the DNA encoding a 5-HT₄ receptor as to permit expression thereof.

20. A plasmid of claim 19 designated pcEXV-S10-87 (ATCC Accession No. 75390).

21. A plasmid of claim 19 designated pcEXV-S10-95 (ATCC Accession No. 75391).

22. A plasmid of claim 19 designated pBluescript-hS10 (ATCC Accession No. 75392).

23. A mammalian cell comprising the plasmid of claim 14.

24. A mammalian cell of claim 23, wherein the mammalian cell is an LM (tk−) cell.

25. A nucleic acid probe comprising a nucleic acid molecule of at least 15 nucleotides capable of specifically hybridizing with a unique sequence included within the sequence of a nucleic acid molecule encoding a mammalian 5-HT₄ receptor.

26. A nucleic acid probe comprising a nucleic acid molecule of at least 15 nucleotides capable of specifically hybridizing with a unique sequence included within the sequence of a nucleic acid molecule encoding a human 5-HT₄ receptor.

27. The nucleic acid probe of claim 25, wherein the nucleic acid is DNA.

28. The nucleic acid probe of claim 26, wherein the nucleic acid is DNA.

29. A mixture of nucleic acid probes in accordance with claim 25, such probes having sequences which differ from one another at predefined positions.

30. An antisense oligonucleotide having a sequence capable of specifically binding to a mRNA molecule encoding a mammalian 5-HT₄ receptor so as to prevent translation of the mRNA molecule.

31. An antisense oligonucleotide capable of specifically binding to a mRNA molecule encoding a human 5-HT₄ receptor so as to prevent translation of the mRNA molecule.

32. An antisense oligonucleotide of claim 30 comprising chemical analogs of nucleotides.

33. A mixture of antisense oligonucleotides according to claim 30, such oligonucleotides having sequences which differ from one another at predefined positions.

34. A method for detecting expression of a mammalian 5-HT₄ receptor, which comprises obtaining RNA from cells or tissue, contacting the RNA so obtained with a nucleic acid probe of claim 25 under hybridizing conditions, detecting the presence of any mRNA hybridized to the probe, the presence of mRNA hybridized to the probe indicating expression of the mammalian 5-HT₄ receptor, and thereby detecting the expression of the mammalian 5-HT₄ receptor.

35. A method for detecting expression of a human 5-HT₄ receptor, which comprises obtaining RNA from cells or tissue, contacting the RNA so obtained with a nucleic acid probe of claim 26 under hybridizing conditions, detecting the presence of any mRNA hybridized to the probe, the presence of mRNA hybridized to the probe indicating expression of the human 5-HT₄ receptor, and thereby detecting the expression of the human 5-HT₄ receptor.

36. A method of detecting expression of a mammalian 5-HT₄ receptor in a cell or tissue by in situ hybridization, which comprises contacting the cell or tissue with a nucleic acid probe of claim 25 under hybridizing conditions, detecting the presence of any mRNA hybridized to the probe, the presence of mRNA hybridized to the probe indicating expression of a mammalian 5-HT₄ receptor, and thereby detecting the expression of a mammalian 5-HT₄ receptor.

37. A method of detecting expression of a human 5-HT₄ receptor in a cell or tissue by in situ hybridization, which comprises contacting the cell or tissue with a nucleic acid probe of claim 26 under hybridizing conditions, detecting the presence of any mRNA hybridized to the probe, the presence of mRNA hybridized to the probe indicating expression of a human 5-HT₄ receptor, and thereby detecting the expression of the human 5-HT₄ receptor.

38. A method of isolating from a gene library a gene encoding a receptor other than the 5-HT₄ receptor which comprises contacting the library under hybridizing conditions with a probe of claim 27 and isolating any gene to which the probe hybridizes.

39. A method of claim 38, which additionally comprises simultaneously contacting the DNA comprising the library under hybridizing conditions with a second nucleic acid probe comprising a sequence capable of hybridizing to a DNA sequence of the complementary strand of the DNA of the gene to which the first probe hybridizes, treating any gene sequence to which both probes hybridized so as to produce multiple copies of the gene sequence, isolating the amplified gene sequence and using the isolated gene sequence as a probe to isolate from a gene library the gene to which the amplified DNA sequence hybridizes.

40. The gene isolated by the method of claim 38 or 39.

41. A synthetic gene which comprises the isolated nucleic acid molecule of claim 1 and at least one regulatory element attached thereto so as to increase the number of RNA molecules transcribed from the synthetic gene.

42. A synthetic gene which comprises the isolated nucleic acid molecule of claim 1 and at least one regulatory element attached thereto so as to decrease the number of RNA molecules transcribed from the synthetic gene.

43. An isolated mammalian 5-HT₄ receptor protein.

44. The receptor protein of claim 43, wherein the mammalian 5-HT₄ receptor protein is a human 5-HT₄ receptor.

45. A method of preparing a mammalian 5-HT₄ receptor of claim 43, which comprises inducing cells to express the mammalian 5-HT₄ receptor and recovering the mammalian 5-HT₄ receptor from the resulting cells.

46. A method of preparing a mammalian 5-HT₄ receptor protein of claim 43, which comprises inserting a nucleic acid molecule encoding the mammalian 5-HT₄ receptor in a suitable vector, inserting the resulting vector in suitable host cell and recovering the mammalian 5-HT₄ receptor produced by the resulting cell.

47. A method of preparing a human 5-HT₄ receptor of claim 44, which comprises inducing cells to express the human 5-HT₄ receptor and recovering the human 5-HT₄ receptor from the resulting cells.

48. A method of preparing a human 5-HT₄ receptor protein of claim 44, which comprises inserting a nucleic acid molecule encoding the human 5-HT₄ receptor in a suitable vector, inserting the resulting vector in suitable host cell and recovering the human 5-HT₄ receptor produced by the resulting cell.

49. An antibody directed to a mammalian 5-HT₄ receptor or to a protein fragment of the mammalian 5-HT₄ receptor.

50. An antibody directed to a human 5-HT₄ receptor or to a protein fragment of the human 5-HT₄ receptor.

51. An antibody of claim 49, wherein the antibody is a monoclonal antibody.

52. An antibody of claim 50, wherein the antibody is a monoclonal antibody.

53. A monoclonal antibody of claim 51, wherein the antibody is directed to an epitope of a mammalian cell-surface 5-HT₄ receptor and having an amino acid sequence substantially the same as the amino acid sequence of a cell-surface epitope of the mammalian 5-HT₄ receptor.

54. A monoclonal antibody of claim 52, wherein the antibody is directed to an epitope of a human cell-surface 5-HT₄ receptor and having an amino acid sequence substantially the same as the amino acid sequence for a cell-surface epitope of the human 5-HT₄ receptor.

55. A pharmaceutical composition comprising an amount of a substance effective to alleviate the abnormalities resulting from overexpression of a human 5-HT₄ receptor and a pharmaceutically acceptable carrier.

56. A pharmaceutical composition comprising an amount of a substance effective to alleviate abnormalities resulting from underexpression of a human 5-HT₄ receptor and a pharmaceutically acceptable carrier.

57. A pharmaceutical composition comprising an effective amount of an oligonucleotide of claim 31 effective to reduce expression of a human 5-HT₄ receptor by passing through a cell membrane and specifically binding with mRNA encoding a human 5-HT₄ receptor in the cell so as to prevent its translation and a pharmaceutically acceptable hydrophobic carrier capable of passing through a cell membrane.

58. A pharmaceutical composition claim 57, wherein the nucleotide is coupled to a substance which inactivates mRNA.

59. A pharmaceutical composition of claim 58, wherein the substance which inactivates the mRNA is a ribozyme.

60. A pharmaceutical composition of claim 58, wherein the pharmaceutically acceptable hydrophobic carrier capable of passing through a cell membrane comprises a structure which binds to a transporter specific for a selected cell type and is thereby taken up by the cells of the selected cell type.

61. A pharmaceutical composition which comprises an amount of the antibody of claim 50 effective to block binding of naturally occurring substrates to a human 5-HT₄ receptor and a pharmaceutically acceptable carrier.

62. A transgenic nonhuman mammal which comprises a nucleic acid molecule of claim 1.

63. A transgenic nonhuman mammal whose genome comprises a nucleic acid molecule of claim 1 so placed as to be transcribed into antisense mRNA complementary to mRNA encoding a human 5-HT₄ receptor and which hybridizes to mRNA encoding a human 5-HT₄ receptor thereby reducing its translation.

64. The transgenic nonhuman mammal of claim 62, wherein the nucleic acid molecule further comprises an inducible promoter.

65. The transgenic nonhuman mammal of claim 62 or 64 wherein the nucleic molecule additionally comprises tissue specific regulatory elements.

66. The transgenic non-human mammal of 62 wherein the transgenic non-human mammal is a mouse.

67. A method for determining the physiological effects of varying the levels of expression of a human 5-HT₄ receptor which comprises producing a transgenic non-human mammal whose levels of expression of a human 5-HT₄ receptor can be varied by use of an inducible promoter.

68. A method for determining the physiological effects of expressing varying levels of a human 5-HT₄ receptor which comprises producing a panel of transgenic non-human mammals each expressing a different amount of a human 5-HT₄ receptor.

69. A method for determining whether a compound not known to be capable of specifically binding to a human 5-HT₄ receptor can specifically bind to the human 5-HT₄ receptor, which comprises contacting a mammalian cell comprising a plasmid adapted for expression in a mammalian cell which plasmid further comprises DNA which expresses a human 5-HT₄ receptor on the cell's surface with the compound under conditions permitting binding of ligands known to bind to a human 5-HT₄ receptor, detecting the presence of any compound bound to the human 5-HT₄ receptor, the presence of bound compound indicating that the compound is capable of specifically binding to the human 5-HT₄ receptor.

70. The method of claim 70, wherein the mammalian cell is a non-neuronal cell.

71. A method of screening compounds to identify drugs which interact with, and specifically bind to, a human 5-HT₄ receptor on the surface of a cell, which comprises contacting a mammalian cell which comprises a plasmid adapted for expression in a mammalian cell which plasmid further comprises DNA which expresses a human 5-HT₄ receptor on the cell's surface with a plurality of compounds, determining those compounds which bind to the human 5-HT₄ receptor expressed on the cell surface of the mammalian cell, and thereby identifying compounds which interact with, and specifically bind to, the human 5-HT₄ receptor.

72. The method of claim 71, wherein the mammalian cell is a non-neuronal cell.

73. A method for determining whether a compound not known to be capable of specifically binding to a human 5-HT₄ receptor can specifically bind to a human 5-HT₄ receptor, which comprises preparing a cell extract from mammalian cells, which comprise a plasmid adapted for expression in a mammal, which plasmid further comprises DNA which expresses a human 5-HT₄ receptor on the cell's surface, isolating a membrane fraction from the cell extract, incubating the compound with the membrane fraction under conditions permitting binding of ligands known to bind to the human 5-HT₄ receptor, detecting the presence of any bound compound, and thereby determining whether the compound is capable of specifically binding to the human 5-HT₄ receptor.

74. The method of claim 73, wherein the mammalian cell is a non-neuronal cell.

75. A method for screening compounds to identify drugs that interact with, and specifically bind to, a human 5-HT₄ receptor, which comprises preparing a cell extract from mammalian cells, which comprise a plasmid adapted for expression in a mammalian cell which plasmid further comprises DNA which expresses a human 5-HT₄ receptor on the cell's surface, isolating a membrane fraction from the cell extract, incubating the membrane fraction with a plurality of compounds, determining those compounds which interact with and bind to the human 5-HT₄ receptor, and thereby identifying compounds which interact with, and specifically bind to, the human 5-HT₄ receptor.

76. The method of claim 75, wherein the mammalian cell is a non-neuronal cell.

77. A method for identifying a compound which is not known to be capable of binding to the human 5-HT₄
receptor activates the human 5-HT₄ receptor on the surface of a mammalian cell or prevents a ligand which does so, which comprises contacting the mammalian cell which cell comprises a plasmid adapted for expression in the mammalian cell such plasmid further comprising DNA which expresses the human 5-HT₄ receptor on the cell surface of the mammalian cell with the compound under conditions permitting activation of blockade of a functional response, determining whether the compound activates the human 5-HT₄ receptor or prevents a ligand which does so, and thereby identifying the compound as a compound which interacts with, and activates the human 5-HT₄ receptor or prevents the activation of the human 5-HT₄ receptor by a ligand which does so.

78. The method of claim 77, wherein the mammalian cell is a non-neuronal cell comprising the cellular components necessary to produce a second messenger and wherein the determination of whether the compound activates of blocks the activation of the human 5-HT₄ comprises detecting the change in the concentration of the second messenger.

79. The method of claim 78, wherein the second messenger is cyclic AMP (cAMP).

80. The method of claim 78, wherein the non-neuronal cell is a COS-7 cell.

81. A method of claim 78, wherein the second messenger is an inositol phosphate metabolite.

82. The method of claim 78, wherein the second messenger is intracellular calcium.

83. A compound identified by the method of claim 69, 73 or 77.

84. A pharmaceutical composition of a drug identified by the method of claim 71 or 75.

85. A method for detecting the presence of a human 5-HT₄ receptor on the surface of a cell, which comprises contacting the cell with an antibody of claim 50, under conditions that permit binding of the antibody to the receptor, detecting the presence of any of the antibody bound to the cell, and thereby the presence of a human 5-HT₄ receptor on the surface of the cell.

86. A method for treating an abnormal condition related to an excess of activity of a human 5-HT₄ receptor, which comprises administering a patient an amount of a pharmaceutical composition of claim 84, effective to reduce 5-HT₄ activity as a result of naturally occurring substrate binding to and activating the 5-HT₄ receptor.

87. The method of treating abnormalities which are alleviated by an increase in the activity of a 5-HT₄ receptor, which comprises administering a patient an amount of a pharmaceutical composition of claim 84, effective to increase the activity of the 5-HT₄ receptor thereby alleviating abnormalities resulting from abnormally low receptor activity.

88. A method for diagnosing a predisposition to a disorder associated with the expression of a human 5-HT₄ receptor allele which comprises:
a. obtaining DNA from subjects suffering from a disorder;
b. performing a restriction digest of the DNA with a panel of restriction enzymes;
c. electrophoretically separating the resulting DNA fragments on a sizing gel;
d. contacting the gel with a nucleic acid probe capable of specifically hybridizing to DNA encoding a human 5-HT₄ receptor and labelled with a detectable marker;
e. detecting the labelled bands which have hybridized to the DNA encoding 5-HT₄ receptor, labelled with the detectable marker to create a unique band pattern specific to the DNA of subjects suffering with the disorder;
f. preparing DNA for diagnosis by steps a-e;
g. comparing the unique band pattern specific to the DNA of patients suffering from the disorder from step e and DNA obtained for diagnosis from step f to determine whether the patterns are the same or different and to diagnose thereby predisposition to the disorder if the patterns are the same.

89. The method of claim 88, wherein a disorder is associated with the expression of a specific human 5-HT₄ receptor allele is diagnosed.

90. A method of identifying a substance capable of alleviating the abnormalities resulting from overexpression of a human 5-HT₄ receptor which comprises administering a substance to the transgenic non-human mammal of claim 67 or 68, and determining whether the substance alleviates the physical and behavioral abnormalities displayed by the transgenic nonhuman mammal as a result of overexpression of the human 5-HT₄ receptor.

91. A method of identifying a substance capable of alleviating the abnormalities resulting from underexpression of a human 5-HT₄ receptor, which comprises administering a substance to the transgenic mammal of claim 62, and determining whether the substance alleviates the physical and behavioral abnormalities displayed by the transgenic nonhuman mammal as a result of underexpression of the human 5-HT₄ receptor.

92. A method of treating abnormalities in a subject, wherein the abnormality is alleviated by the reduced expression of a human 5-HT₄ receptor which comprises administering to a subject an effective amount of the pharmaceutical composition of claim 55, 57, 83 or 84 effective to reduce expression of the human 5-HT₄ receptor.

93. A method of treating abnormalities resulting from underexpression of a human 5-HT₄ receptor which comprises administering to a subject an amount of a pharmaceutical composition of claim 56, 83 or 84 effective to alleviate abnormalities resulting from underexpression of the human 5-HT₄ receptor.
